(19) United States
(12) Patent Application Publication
(10) Pub. No.: US 2023/0002770 A1
(43) Pub. Date: Jan. 5, 2023

(54) IL-34 ANTISENSE AGENTS AND METHODS OF USING SAME

(71) Applicant: Nogra Pharma Limited, Dublin 2 (IE)

(72) Inventors: Francesca Viti, Salorino (CH); Marie McNulty, Dublin (IE); Salvatore Bellinvia, Mendrisio (CH)

(21) Appl. No.: 17/755,943

(22) PCT Filed: Nov. 16, 2020
(86) PCT No.: PCT/EP2020/082281

§ 371 Date: May 12, 2022

Related U.S. Application Data
(60) Provisional application No. 62/935,819, filed on Nov. 15, 2019.

(51) Int. Cl. C12N 15/113; A61P 1/00

(52) U.S. Cl. CPC C12N 15/1136; A61P 1/00; C12N 2310/315; C12N 2310/3341; C12N 2310/3231; C12N 2310/11; C12N 2310/14

(57) ABSTRACT

Disclosed herein are IL-34 inhibitors, including IL-34 antisense oligonucleotide sequences, and methods for treating inflammatory diseases, such as an inflammatory bowel disease, and/or fibrosis, associated with elevated activity or expression of IL-34. Also disclosed are pharmaceutical compositions containing an IL-34 inhibitor, for example, a IL-34 antisense oligonucleotide, useful for treating inflammatory diseases and/or fibrosis and manufacture of medicaments containing a disclosed IL-34 inhibitor to be used in treating inflammatory diseases and/or fibrosis.

### CROSS REFERENCE TO RELATED APPLICATIONS

This application claims the benefit and priority to U.S. Provisional Patent Application No. 62/935,819, filed Nov. 15, 2019, the disclosure of which is hereby incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on Nov. 13, 2020, is named GIU-060 WO SL ST25.txt and is 21,080 bytes in size.

### BACKGROUND

Interleukin-34 (IL-34) is a recently discovered cytokine functionally overlapping with macrophage colony stimulating factor (M-CSF, also known as MCSF1 and MCSF-1), a mediator of inflammation and osteoclastogenesis in bone-degenerative diseases such as rheumatoid arthritis. Inflammatory diseases, both acute and chronic, are an important disease category that is still not completely understood.

For example, inflammatory bowel disease is a chronic inflammatory disorder of the gastrointestinal tract suffered by approximately 1.4 million patients in the United States. It is one of the five most prevalent gastrointestinal disease burdens in the United States, with an overall health care cost of more than $1.7 billion. Each year in the United States, inflammatory bowel disease accounts for more than 700,000 physician visits, 100,000 hospitalizations, and disability in 119,000 patients. No medical cure currently exists, so disease management requires a lifetime of care.

The two most common forms of inflammatory bowel disease are Crohn's disease and ulcerative colitis. Although Crohn's disease can affect the entire gastrointestinal tract, it primarily affects the ileum (the distal or lower portion of the small intestine) and the large intestine. Ulcerative colitis primarily affects the colon and the rectum. The etiology of inflammatory bowel disease is not completely understood, although both environmental and genetic factors are believed to play a role in the disease. Environmental components may include alterations in flora of the gut which are affected by exposure to ingested foods and medications.

Inflammatory bowel disease is associated with abdominal pain, vomiting, diarrhea, rectal bleeding, severe cramps, muscle spasms, weight loss, malnutrition, fever, and anemia. Patients with inflammatory bowel disease may also suffer from skin lesions, joint pain, eye inflammation, and liver disorders, and children suffering from ulcerative colitis may suffer from growth defects. Although rarely fatal, these symptoms decrease quality of life for patients.

Thus, there is a pressing need to develop reliable methods of treating inflammatory disorders such as inflammatory bowel disease. There is a further need to identify methods of treatment that provide effective and permanent relief from symptoms across a broad spectrum of patients and which are not associated with negative side effects or cycles of inflammation and remission.

### SUMMARY

Described herein are agents, for example, IL-34 antisense oligonucleotides, that inhibit IL-34 gene expression. In some embodiments, the invention provides an IL-34 antisense oligonucleotide, or a pharmaceutically acceptable salt thereof, that includes the antisense oligonucleotide sequence of any of SEQ ID NOs:1-8 described herein. For example, an IL-34 antisense oligonucleotide of the invention can be an antisense oligonucleotide that includes the sequence: 5′-CTCACCAAGACCCACAG-3′ (SEQ ID NO:1), wherein at least one nucleoside is a chemically modified nucleoside and/or at least one linkage is a modified internucleoside linkage; 5′-GGCTTTGGGCCGCACCAGCT-3′ (SEQ ID NO:2), wherein at least one nucleoside is a chemically modified nucleoside and/or at least one linkage is a modified internucleoside linkage; 5′-CTTTGGGCCGCACCAGCTTC-3′ (SEQ ID NO:3), wherein at least one nucleoside is a chemically modified nucleoside and/or at least one linkage is a modified internucleoside linkage; 5′-TGGGCCGCACCAGCTTCAGG-3′ (SEQ ID NO:4), wherein at least one nucleoside is a chemically modified nucleoside and/or at least one linkage is a modified internucleoside linkage; 5′-TCCATGACCCGGAAGCAGTT-3′ (SEQ ID NO:5), wherein at least one nucleoside is a chemically modified nucleoside and/or at least one linkage is a modified internucleoside linkage; or 5′-TGTTTCATGTACTGAAG-3′ (SEQ ID NO:6), wherein at least one nucleoside is a chemically modified nucleoside and/or at least one linkage is a modified internucleoside linkage, or a pharmaceutically acceptable salt thereof. In some embodiments, the antisense oligonucleotide comprises the sequence of SEQ ID NO:3, wherein at least one nucleoside is a chemically modified nucleotide and/or at least one linkage is a modified internucleoside linkage. In some embodiments, the antisense oligonucleotide comprises the sequence of SEQ ID NO:3, wherein at least one cytidine is chemically modified. In some embodiments, the antisense oligonucleotide comprises the sequence of 5′-CTTTGGGCXGCACCAGCTTC-3′ (SEQ ID NO:7), wherein X is 5-methylcytidine. In some embodiments, the antisense oligonucleotide comprises the sequence of SEQ ID NO:5, wherein at least one cytidine is chemically modified; optionally, wherein the cytidine at position 10 of SEQ ID NO:5 is chemically modified and the nucleotide sequence is 5′-TCCATGACCXGGAAGCAGTT-3′ (SEQ ID NO:8), and wherein X is 5-methylcytidine.

In some embodiments, an antisense oligonucleotide described herein can include ribonucleotides and/or deoxyribonucleotides. For example, in some embodiments described herein, an IL-34 antisense oligonucleotide, for example, an IL-34 antisense oligonucleotide of any one of SEQ ID NOs:1-8, can include one or more ribonucleotides. In some embodiments described herein, an IL-34 antisense oligonucleotide, for example, an IL-34 antisense oligonucleotide of any one of SEQ ID NOs:1-8, can include one or more deoxyribonucleotides. In some embodiments described herein, an IL-34 antisense oligonucleotide can include a mixture of ribonucleotides and deoxyribonucleotides.

In some embodiments described herein, an IL-34 antisense oligonucleotide can include one or more locked nucleic acid (“LNA”) nucleotides. An IL-34 antisense oligonucleotide described herein, for example, an IL-34 antisense oligonucleotide of any one of SEQ ID NOs:1-8, can include one or more LNA nucleotides, for example, 1, 2, 3, 4, 5, 6, 7, 8, 9, 10, 11, 12, 13, 14, 1-5, 5-10, 5-12, or more LNA nucleotides. In some embodiments, each of the nucleotides of an IL-34 antisense oligonucleotide described herein is an LNA nucleotide.

In particular embodiments, an IL-34 antisense oligonucleotide includes LNA nucleotides and the sequence of the IL-34 antisense oligonucleotide is selected from one of the following:
5′-cttTGGGCXGCACCAGCttc-3′ (SEQ ID NO:9), wherein c is LNA cytidine, t is LNA thymidine, and X is 5-methylcytidine;
5′-ctttGGGCXGCACCAGcttc-3′ (SEQ ID NO:10), wherein c is LNA cytidine, t is LNA thymidine, and X is 5-methylcytidine;
5′-cttTGGGCcgCACCAGCttc-3′ (SEQ ID NO:11), wherein c is LNA cytidine, t is LNA thymidine, and g is LNA guanosine;
5′-cttTGGGCcGCACCAGCttc-3′ (SEQ ID NO:12), wherein c is LNA cytidine and t is LNA thymidine;
5′-ggcXGCACCAGCttc-3′ (SEQ ID NO:13), wherein c is LNA cytidine, t is LNA thymidine, g is LNA guanosine, and X is 5-methylcytidine;
5′-cttTGGGCXGCACcag-3′ (SEQ ID NO:14), wherein c is LNA cytidine, t is LNA thymidine, g is LNA guanosine, a is LNA adenosine, and X is 5-methylcytidine; and
5′-tgaCCXGGAAGCAgtt-3′ (SEQ ID NO:15), wherein a is LNA adenosine, t is LNA thymidine, g is LNA guanosine, and X is 5-methylcytidine,
   or a pharmaceutically acceptable salt thereof.

Additionally, in embodiments described herein, IL-34 antisense oligonucleotides can include chemically modified nucleosides, for example, 2′-O-methyl (“2′-OMe”) ribonucleosides, for example, 2′-O-methylcytidine, 2′-O-methylguanosine, 2′-O-methylthymidine, 2′-O-methyluridine, and/or 2′-O-methyladenosine. IL-34 antisense oligonucleotides described herein, can also include one or more chemically-modified bases, including a 5-methylpyrimidine, for example, 5-methylcytosine; and/or a 5-methylpurine, for example, 5-methylguanine. IL-34 antisense oligonucleotides described herein, can also include any of the following chemically-modified nucleosides: 5-methyl-2′-O-methylcytidine, 5-methyl-2′-O-methylthymidine, 5-methylcytidine, 5-methyluridine, and/or 5-methyl-2′-deoxycytidine. Additionally, IL-34 antisense oligonucleotides described herein can include one or more 2′-O-(2-methoxyethyl) (“2′-MOE”) nucleosides, 2′-deoxy-2′-fluoro nucleosides, 2′-fluoro-β-D-arabinonucleosides, bridged nucleic acids, LNA nucleotides, constrained ethyl′ (cET) nucleic acids, tricyclo-DNAs (tcDNA), 2′-0,4′-C-ethylene linked nucleic acids (ENA), and/or peptide nucleic acids (PNA). Embodiments described herein include IL-34 antisense oligonucleotides of any of SEQ ID NOs:1-15, or a pharmaceutically acceptable salt thereof, that include one or more of any of the aforementioned chemically-modified nucleosides. In some embodiments, at least one nucleoside of the IL-34 antisense oligonucleotide sequence is a chemically-modified nucleoside. For example, in some embodiments, 1, 2, 3, 4, 5, 6, 7, 8, 9, 10, 11, 12, 13, 14, 15, 16, 17, 18, 19, 1-5, 1-10, 1-14, 1-15, 1-16, 1-19, 5-10, 5-14, 5-15, 5-19, 10-14, 10-15, or 10-19 nucleosides of the IL-34 antisense oligonucleotide sequence are nucleosides.

In particular embodiments, an IL-34 antisense oligonucleotide described herein includes 2′-MOE nucleosides. In some embodiments, the sequence of the IL-34 antisense oligonucleotide is selected from one of the following:
5′-CxTxTxTxGxGGCXGCACCAGxCxTxTxCx-3′ (SEQ ID NO:16), wherein Cx is 2′-O-(2-methoxyethyl)cytidine, Tx is 2′-O-(2-methoxyethyl)thymidine, Gx is 2′-O-(2-methoxyethyl)guanosine, and X is 5-methylcytidine;
5′-TxCxCxAxTxGACCXGGAAGCxAxGxTxTx-3′ (SEQ ID NO:17), wherein Cx is 2′-O-(2-methoxyethyl)cytidine, Tx is 2′-O-(2-methoxyethyl)thymidine, Gx is 2′-O-(2-methoxyethyl)guanosine, Ax is 2′-O-(2-methoxyethyl)adenosine, and X is 5-methylcytidine;
5′-CxTxTxTxGxGxGCXGCACCAxGxCxTxTxCx-3′ (SEQ ID NO:18), wherein Cx is 2′-O-(2-methoxyethyl)cytidine, Tx is 2′-O-(2-methoxyethyl)thymidine, Gx is 2′-O-(2-methoxyethyl)guanosine, Ax is 2′-O-(2-methoxyethyl)adenosine, and X is 5-methylcytidine;
5′-TxCxCxAxTxGxACCXGGAAGxCxAxGxTxTx-3′ (SEQ ID NO:19), wherein Cx is 2′-O-(2-methoxyethyl)cytidine, Tx is 2′-O-(2-methoxyethyl)thymidine, Gx is 2′-O-(2-methoxyethyl)guanosine, Ax is 2′-O-(2-methoxyethyl)adenosine, and X is 5-methylcytidine;
5′-CxTxTxTxGxGxGxCxXGxCxAxCxCxAxGxCxTxTxCx-3′ (SEQ ID NO:20), wherein Cx is 2′-O-(2-methoxyethyl)cytidine, Tx is 2′-O-(2-methoxyethyl)thymidine, Gx is 2′-O-(2-methoxyethyl)guanosine, Ax is 2′-O-(2-methoxyethyl)adenosine, and X is 5-methylcytidine; and
5′-TxCxCxAxTxGxAxCxCxXGxGxAxAxGxCxAxGxTxTx-3′ (SEQ ID NO:21), wherein Cx is 2′-O-(2-methoxyethyl)cytidine, Tx is 2′-O-(2-methoxyethyl)thymidine, Gx is 2′-O-(2-methoxyethyl)guanosine, Ax is 2′-O-(2-methoxyethyl)adenosine, and X is 5-methylcytidine,
   or a pharmaceutically acceptable salt thereof.

Additionally, in some embodiments, an IL-34 antisense oligonucleotide described herein includes 2′-OMe nucleosides. In some embodiments, the sequence of the IL-34 antisense oligonucleotide is selected from one of the following:
5′-CyTyTyTyGyGGCXGCACCAGyCyTyTyCy-3′ (SEQ ID NO:22), wherein Cy is 2′-O-methylcytidine, Ty is 2′-O-methylthymidine, Gy is 2′-O-methylguanosine, and X is 5-methylcytidine; and
5′-TyCyCyAyTyGACCXGGAAGCyAyGyTyTy-3′ (SEQ ID NO:23), wherein Cy is 2′-O-methylcytidine, Ty 2′-O-methylthymidine, Gy is 2′-O-methylguanosine, Ay is 2′-O-methyladenosine, and X is 5-methylcytidine,
   or a pharmaceutically acceptable salt thereof.

In some embodiments, an IL-34 antisense oligonucleotide described herein includes one or more modified internucleoside linkages. For example, in some embodiments, the IL-34 antisense oligonucleotide is an antisense oligonucleotide wherein at least one internucleoside linkage of the sequence is a phosphorothioate linkage, a phosphorodithioate linkage, a phosphotriester linkage, an alkylphosphonate linkage, an aminoalkylphosphotriester linkage, an alkylene phosphonate linkage, a phosphinate linkage, a phosphoramidate linkage, and an aminoalkylphosphoramidate linkage, a thiophosphoramidate linkage, thionoalkylphosphonate linkage, a thionoalkylphosphotriester linkage, a thiophosphate linkage, a selenophosphate linkage, or a boranophosphate linkage. In particular embodiments, at least one internucleoside linkage of the IL-34 antisense oligonucleotide sequence is a phosphorothioate linkage. For example, in some embodiments, 1, 2, 3, 4, 5, 6, 7, 8, 9, 10, 11, 12, 13, 14, 15, 16, 17, 18, 19, 1-5, 1-10, 1-14, 1-15, 1-16, 1-19, 5-10, 5-14, 5-15, 5-19, 10-14, 10-15, or 10-19 internucleoside linkages of the IL-34 antisense oligonucleotide sequence are phosphorothioate linkages. In some embodiments, all of the internucleoside linkages of the antisense oligonucleotide sequence are phosphorothioate linkages.

The number of nucleotides included in IL-34 antisense oligonucleotides described herein may vary. For example, in some embodiments, the antisense oligonucleotide is from 15-20, 15-25, 15-30, 15-35, 20-25, 20-30, 20-35, 25-30, 25-35, or 30-35 nucleotides in length. In particular embodiments, the antisense oligonucleotide is from 15-25 nucleotides in length. In particular embodiments, the antisense oligonucleotide is from 20-25 nucleotides in length. In some embodiments, the antisense oligonucleotide is 15, 16, 17, 18, 19, 20, 21, 22, 23, 24, 25, 26, 27, 28, 29, 30, 31, 32, 33, 34, or 35 nucleotides in length. In some embodiments an antisense oligonucleotide described herein includes a maximum number of nucleotides. In some embodiments, an IL-34 antisense oligonucleotide is no more than 15, 16, 17, 18, 19, 20, 21, 22, 23, 24, 25, 30, or 35 nucleotides in length.

In an embodiment, provided herein is a compound comprising an antisense oligonucleotide of any one of SEQ ID NOs:1-23, or a pharmaceutically acceptable salt thereof. For example, in some embodiments, the compound comprises an antisense oligonucleotide of SEQ ID NO:3, wherein at least one cytidine is chemically modified. In some embodiments, the compound comprises an antisense oligonucleotide of SEQ ID NO:7, wherein X is 5-methylcytidine.

In embodiments described herein, a nucleoside of an IL-34 antisense oligonucleotide, for example, an IL-34 antisense oligonucleotide of any one of SEQ ID NOs:1-15, or a pharmaceutically acceptable salt thereof, can be substituted with a chemically modified nucleoside. For example, in some embodiments, one or more cytidines of an IL-34 antisense oligonucleotide are replaced with 5-methylcytidine. In some embodiments, an antisense oligonucleotide can include one or more modified nucleotides, for example, 5-methyl-2′-deoxycytidine. For example, in varying embodiments, an IL-34 antisense oligonucleotide of the present disclosure includes or can include nucleotides including deoxycytidine and/or 5-methyl-2′-deoxycytidine, including, but not limited to, 5-methyl-2′-deoxycytidine 5′-monophosphate and 5-methyl-2′-deoxycytidine-5′-monophosphorothioate. For example, in embodiments described herein, one or more cytidine nucleosides of an IL-34 antisense oligonucleotide of any one of SEQ ID NOs:1-15, or a pharmaceutically acceptable salt thereof, can each be substituted with 5-methylcytidine. In some particular embodiments, one cytidine nucleoside of an IL-34 antisense oligonucleotide described herein is substituted with 5-methylcytidine.

Also described herein are methods of treatment wherein an IL-34 antisense oligonucleotide is administered to a patient in need thereof. For example, described herein are methods of treating an inflammatory disease, comprising administering to a patient in need thereof an effective amount of an IL-34 antisense oligonucleotide described herein, for example, an IL-34 antisense oligonucleotide of any one of SEQ ID NOs:1-23. Also described herein is a method of inhibiting inflammatory cytokine production in cells of a patient suffering from an inflammatory disease, comprising administering an effective amount of an IL-34 antisense oligonucleotide described herein, for example, an IL-34 antisense oligonucleotide of any one of SEQ ID NOs:1-15.

Also described herein is a method of reducing or inhibiting an IL-34 mediated inflammatory response in a cell or cells of a patient suffering from an inflammatory disease, comprising administering an effective amount of an IL-34 antisense oligonucleotide, for example, an IL-34 antisense oligonucleotide of any one of SEQ ID NOs:1-15. Also described herein, is a method of treating an inflammatory disease associated with altered IL-34 expression in a patient in need thereof, the method comprising administering an effective amount of an IL-34 antisense oligonucleotide. For example, described herein is a method of treating an inflammatory disease associated with increased IL-34 expression in a patient in need thereof, where the method includes administering an effective amount of an IL-34 antisense oligonucleotide of any one of SEQ ID NOs:1-23, or a pharmaceutically acceptable salt thereof. Increased IL-34 expression may be relative to a control level of IL-34 expression, for example, a mean or median level of IL-34 measured in a healthy control patient or a cohort of healthy control patients, or a level of IL-34 measured in the patient prior to onset or detection of the disease associated with increased IL-34 expression.

Also described herein is a method of treating an inflammatory disease associated with altered IL-34 expression in a patient in need thereof, comprising administering an effective amount of an IL-34 antisense oligonucleotide, or a pharmaceutically acceptable salt thereof, for example, an IL-34 antisense oligonucleotide of any one of SEQ ID NOs:1-23.

Also described herein is a method of inhibiting IL-34-mediated macrophage colony-stimulating factor receptor (M-CSFR-1) signaling in cells of a patient suffering from an inflammatory disease, comprising administering an effective amount of an IL-34 antisense oligonucleotide, or a pharmaceutically acceptable salt thereof, for example, an IL-34 antisense oligonucleotide of any one of SEQ ID NOs:1-23.

Also described herein is a method of reducing or eliminating a fibrotic stricture in a patient suffering from an inflammatory disease, comprising administering an effective amount of an IL-34 antisense oligonucleotide, or a pharmaceutically acceptable salt thereof, for example, an IL-34 antisense oligonucleotide of any one of SEQ ID NOs:1-23. In some embodiments, the fibrotic stricture is located in the intestine, for example, the large intestine or the small intestine. In particular embodiments, the fibrotic stricture is located in the large intestine. In some embodiments, the fibrotic stricture is localized to one or more portions of the large and/or small intestine, for example, the cecum, the ileum, the ascending colon, the transverse colon, the descending colon, the sigmoid colon, the rectum, the anus, the duodenum, and/or the jejunum.

Methods described herein may be used to treat one or more inflammatory diseases including, but not limited to, an inflammatory bowel disease, rheumatoid arthritis, psoriasis, osteoarthritis, diabetes (Type I and II), tissue or organ rejection, multiple sclerosis, periodontal inflammation, periodontitis, pigmented villonodular synovitis, hepatitis, sinusitis, colon cancer, colorectal cancer, colitis-associated colon cancer, sporadic colorectal cancer, coronary artery disease, Sjogren's syndrome (SS), obesity, chronic inflammation, pulmonary sarcoidosis, skin lesions, a CNS inflammatory disease, and an autoimmune disease. In some embodiments, methods of the invention may be used to treat asthma, chronic obstructive pulmonary disease (COPD), or idiopathic pulmonary fibrosis (IPF). In some embodiments, the inflammatory disease is an inflammatory bowel disease. In some embodiments, the inflammatory bowel disease is Crohn's disease, gastroduodenal Crohn's disease, Crohn's (granulomatous) colitis, inflammatory Crohn's disease, fibrostricturing Crohn's disease, ulcerative colitis, collagenous colitis, lymphocytic colitis, ischaemic colitis, diversion colitis, Behçet's disease, microscopic colitis, ulcerative proctitis, proctosigmoiditis, jejunoileitis, left-sided colitis, pancolitis, ileocolitis, ileitis, or indeterminate colitis. In particular embodiments, the inflammatory bowel disease is inflammatory Crohn's disease or fibrostricturing Crohn's disease.

In some embodiments, the IL-34 antisense oligonucleotide, or a pharmaceutically acceptable salt thereof, is administered topically, parenterally, orally, pulmonarily, intratracheally, intranasally, transdermally, or intraduodenally. In a particular embodiment, the IL-34 antisense oligonucleotide, or a pharmaceutically acceptable salt thereof, is administered orally.

In some embodiments, the patient in need of treatment is a human.

Also described herein is a pharmaceutically acceptable composition comprising an IL-34 antisense oligonucleotide described herein, for example, an IL-34 antisense oligonucleotide, or a pharmaceutically acceptable salt thereof, of any one of SEQ ID NOs:1-23, and a pharmaceutically acceptable excipient. In some embodiments, the pharmaceutical composition is suitable for topical, parenteral, oral, pulmonary, intratracheal, intranasal, transdermal, or intraduodenal administration.

Also described herein is an IL-34 antisense oligonucleotide for use as a medicament. For example, an IL-34 antisense oligonucleotide of any one of SEQ ID NOs:1-23 can be for use as a medicament.

Also described herein is use of an IL-34 antisense oligonucleotide, for example, an IL-34 antisense oligonucleotide of any one of SEQ ID NOs:1-23, in the manufacture of a medicament for the treatment of an inflammatory disease. In some embodiments, the inflammatory disease is an inflammatory bowel disease, rheumatoid arthritis, psoriasis, osteoarthritis, diabetes (Type I and II), tissue or organ rejection, multiple sclerosis, periodontal inflammation, periodontitis, pigmented villonodular synovitis, hepatitis, sinusitis, colon cancer, colorectal cancer, colitis-associated colon cancer, sporadic colorectal cancer, coronary artery disease, Sjogren's syndrome (SS), obesity, chronic inflammation, pulmonary sarcoidosis, skin lesions, a CNS inflammatory disease, or an autoimmune disease. Also described herein is use of an IL-34 antisense oligonucleotide in the manufacture of a medicament for the treatment of asthma, chronic obstructive pulmonary disease (COPD), or idiopathic pulmonary fibrosis (IPF). In some embodiments, the inflammatory disease is an inflammatory bowel disease. In some embodiments, the inflammatory bowel disease is Crohn's disease, inflammatory Crohn's disease, fibrostricturing Crohn's disease, gastroduodenal Crohn's disease, Crohn's (granulomatous) colitis, ulcerative colitis, collagenous colitis, lymphocytic colitis, ischaemic colitis, diversion colitis, Behçet's disease, microscopic colitis, ulcerative proctitis, proctosigmoiditis, jejunoileitis, left-sided colitis, pancolitis, ileocolitis, ileitis, or indeterminate colitis. In some embodiments, the inflammatory bowel disease is inflammatory Crohn's disease or fibrostricturing Crohn's disease.

Also described herein is an IL-34 antisense oligonucleotide, for example, an IL-34 antisense oligonucleotide of any one of SEQ ID NOs:1-23, for use in the treatment of an inflammatory disease. In some embodiments, the inflammatory disease is an inflammatory bowel disease, rheumatoid arthritis, psoriasis, osteoarthritis, diabetes (Type I and II), tissue or organ rejection, multiple sclerosis, periodontal inflammation, periodontitis, pigmented villonodular synovitis, hepatitis, sinusitis, colon cancer, colorectal cancer, colitis-associated colon cancer, sporadic colorectal cancer, coronary artery disease, Sjogren's syndrome (SS), obesity, chronic inflammation, pulmonary sarcoidosis, skin lesions, a CNS inflammatory disease, or an autoimmune disease. Also described herein is an IL-34 antisense oligonucleotide for use in the treatment of asthma, chronic obstructive pulmonary disease (COPD), or idiopathic pulmonary fibrosis (IPF). In some embodiments, the inflammatory disease is an inflammatory bowel disease. In some embodiments, the inflammatory bowel disease is Crohn's disease, inflammatory Crohn's disease, fibrostricturing Crohn's disease, gastroduodenal Crohn's disease, Crohn's (granulomatous) colitis, ulcerative colitis, collagenous colitis, lymphocytic colitis, ischaemic colitis, diversion colitis, Behçet's disease, microscopic colitis, ulcerative proctitis, proctosigmoiditis, jejunoileitis, left-sided colitis, pancolitis, ileocolitis, ileitis, or indeterminate colitis. In some embodiments, the inflammatory bowel disease is inflammatory Crohn's disease or fibrostricturing Crohn's disease.

Also described herein is a method of inhibiting IL-34 expression in a cell of a subject, comprising administering to the subject a pharmaceutically effective amount of a pharmaceutical preparation that includes an IL-34 antisense oligonucleotide, or a pharmaceutically acceptable salt thereof, for example, an antisense oligonucleotide of any one of SEQ ID NOs:1-23. Also described herein is a method of inhibiting expression of one or more collagens in a cell of a subject, comprising administering to the subject a pharmaceutically effective amount of a pharmaceutical preparation comprising an IL-34 antisense oligonucleotide, or a pharmaceutically acceptable salt thereof, for example, an antisense oligonucleotide of any one of SEQ ID NOs:1-23. In some embodiments of the aforementioned methods, the cell is an intestinal cell, for example, an intestinal stromal cell, an intestinal epithelial cell, an intestinal stem cell, a secretory cell, an enterocyte, a Goblet cell, an enteroendocrine cell, a Paneth cell, a transit amplifying cell, a microfold cell, a cup cell, or a tuft cell. In some embodiments, the cell forms part of an intestinal fibrostricture. In the aforementioned embodiments, subject can be a subject in need of treatment of an inflammatory disease or fibrosis, for example, inflammatory Crohn's disease or fibrostricturing Crohn's disease. In some embodiments, methods described herein can be used to inhibit expression of one or more collagens in a cell of a subject, for example, a type I collagen, a type II collagen, a type III collagen, a type IV collagen, a type V collagen, a type VI collagen, a type VII collagen, a type VIII collagen, a type IX collagen, a type X collagen, a type XI collagen, a type XII collagen, a type XIII collagen, a type XIV collagen, a type XV collagen, a type XVI collagen, a type XVII collagen, a type XVIII collagen, a type XIX collagen, a type XX collagen, a type XXI collagen, a type XXII collagen, a type XXIII collagen, a type XXIV collagen, a type XXV collagen, a type XXVI collagen, a type XXVII collagen, a type XXVIII collagen, or a type XXIX collagen. For example, in embodiments described herein, the collagen is COL1A1, COL1A2II, COL2A1, COL3A1, COL4A1, COL4A2, COL4A3, COL4A4, COL4A5, COL4A6, COL5A1, COL5A2, COL5A3, COL6A1, COL6A2, COL6A3, COL6A5, COL7A1, COL8A1, COL8A2, COL9A1, COL9A2, COL9A3, COL10A1, COL11A1, COL11A2, COL12A1, COL13A1, COL14A1, COL15A1, COL16A1, COL17A1, COL18A1, COL19A1, COL20A1, COL21A1, COL22A1, COL23A1, COL24A1, COL25A1, EMID2, COL27A1, COL28A1, or COL29A1. In particular embodiments, the collagen is COL1A1 (collagen 1A), COL3A1 (collagen 3A), or a mixture thereof. In certain embodiments, the pharmaceutical preparation can be administered orally. In certain embodiments, the subject is a human.

In various embodiments, the invention is a method of treating or preventing fibrosis, for example, intestinal fibrosis, pulmonary fibrosis, or liver fibrosis, or preventing collagen deposition, comprising inhibiting IL-34 in a patient suffering from fibrosis. The invention also can be a method of treating or preventing fibrosis or preventing collagen deposition, where the method comprises inhibiting IL-34 in a cell, for example, an intestinal cell. In some embodiments, the invention can be a method of treating or preventing fibrosis or preventing collagen deposition in a patient, where the method comprises administering to the patient an effective amount of a specific inhibitor of IL-34, for example, an IL-34 antisense oligonucleotide, for example, an IL-34 antisense oligonucleotide that includes the nucleotide sequence of any one of SEQ ID NOs:1-23, or a pharmaceutically acceptable salt thereof.

Also described herein is use of an IL-34 antisense oligonucleotide in the manufacture of a medicament for the treatment of fibrosis. In some embodiments, the fibrosis is intestinal fibrosis. In some embodiments, the fibrosis is pulmonary fibrosis. In other embodiments, the fibrosis is renal fibrosis, cardiac fibrosis, endomyocardial fibrosis, myelofibrosis, retroperitoneal fibrosis, or nephrogenic systemic fibrosis.

Also described herein is an IL-34 antisense oligonucleotide for use in the treatment of fibrosis. In some embodiments, the fibrosis is intestinal fibrosis. In some embodiments, the fibrosis is pulmonary fibrosis. In other embodiments, the fibrosis is renal fibrosis, cardiac fibrosis, endomyocardial fibrosis, myelofibrosis, retroperitoneal fibrosis, or nephrogenic systemic fibrosis.

In some embodiments, the patient suffering from fibrosis, for example, intestinal fibrosis, has previously suffered from an inflammatory bowel disease or is suffering from an inflammatory bowel disease, or an inflammatory bowel disease preceded intestinal fibrosis in the patient. In some embodiments, the inflammatory bowel disease is inflammatory Crohn's disease or fibrostricturing Crohn's disease.

In some embodiments, the patient suffering from intestinal fibrosis previously suffered from colitis or is suffering from colitis, or colitis preceded intestinal fibrosis in the patient. In some embodiments, the colitis is acute colitis. In some embodiments, the colitis is chronic colitis.

In some embodiments of the invention the patient is a mammal, for example, a primate, for example, a human.

It will be appreciated that the IL-34 antisense oligonucleotide administered to the patient having fibrosis in methods of the invention described herein, can be administered by various administration routes. In various embodiments, the IL-34 antisense oligonucleotide can be administered by one or several routes, including orally, topically, parenterally, e.g., by subcutaneous injection, by inhalation spray, or rectally. The term parenteral as used herein includes subcutaneous injections, intrapancreatic administration, and intravenous, intramuscular, intraperitoneal, and intrasternal injection or infusion techniques. In a preferred embodiment, the IL-34 antisense oligonucleotide can be administered orally to the patient having fibrosis, for example, intestinal fibrosis or pulmonary fibrosis.

The invention can provide methods that include administration of a IL-34 antisense oligonucleotide capable of targeting IL-34 RNA for degradation, interfering with RNA splicing, or preventing IL-34 gene expression or protein translation. The IL-34 antisense oligonucleotides of the invention can target various regions of mouse and/or human IL-34 mRNA for binding. The human IL-34 mRNA sequence is the sequence of NCBI Reference Sequence: NM_001172771.1 (SEQ ID NO:24), NM_001172772.1 (SEQ ID NO:25), or NM_152456.2 (SEQ ID NO:26). The mouse IL-34 mRNA sequence is the sequence of NCBI Reference Sequence: NM_001135100.2 (SEQ ID NO:27) or NM_029646.3 (SEQ ID NO:28).

The sequence of the IL-34 antisense oligonucleotide may be selected from multiple sequences capable of targeting IL-34 RNA. In some embodiments of the invention, the antisense oligonucleotide is an antisense oligonucleotide phosphorothioate, i.e., an oligonucleotide where at least some of the internucleotide linkages are phosphorothioate linkages suitable for delivery to cells of a patient. Additionally, antisense oligonucleotides of the invention can include modified nucleotides, for example, nucleotides containing modified bases, for example, 5-methyl-2′-deoxycytidine.

In some embodiments of the invention, a method of treating fibrosis, for example, intestinal fibrosis, preventing intestinal fibrosis or pulmonary fibrosis, or preventing collagen deposition in a patient includes administering a pharmaceutical composition, for example, a pharmaceutical composition comprising a specific inhibitor of IL-34, for example, an IL-34 antisense oligonucleotide, and a pharmaceutically acceptable excipient. In some embodiments, the pharmaceutical composition is administered parenterally. In some embodiments, the pharmaceutical composition is administered orally. In some embodiments, the pharmaceutical composition includes an enteric coating, for example, an enteric coating comprising an ethylacrylate-methacrylic acid copolymer.

In embodiments of the invention, methods of treating and/or preventing an inflammatory disease (for example, inflammatory Crohn's disease or fibrostricturing Crohn's disease), treating, reducing, and/or eliminating fibrotic strictures in a patient (for example, in a patient suffering from fibrostricturing Crohn's disease), treating fibrosis (for example, intestinal fibrosis or pulmonary fibrosis), preventing fibrosis (for example, intestinal fibrosis or pulmonary fibrosis), or treating, ameliorating, or preventing collagen deposition in a patient include administering varying amounts of an IL-34 antisense oligonucleotide, or a pharmaceutically acceptable salt thereof, or a pharmaceutical composition comprising an IL-34 antisense oligonucleotide, for example, an IL-34 antisense oligonucleotide that includes a sequence of one of SEQ ID NOs:1-23, or a pharmaceutically acceptable salt thereof. In some embodiments, methods of the invention include administering at least 1 μg, at least 5 μg, at least 10 μg, at least, 20 μg, at least, 30 μg, at least, 40 μg, at least, 50 μg, at least, 60 μg, at least, 70 μg, at least, 80 μg, at least, 90 or at least 100 μg of the antisense oligonucleotide. In some embodiments, methods of the invention include administering from 35 mg to 500 mg, from 1 mg to 10 mg, from 10 mg to 20 mg, from 20 mg to 30 mg, from 30 mg to 40 mg, from 40 mg to 50 mg, from 50 mg to 60 mg, form 60 mg to 70 mg, from 70 mg to 80 mg, from 80 mg to 90 mg, from 90 mg to 100 mg, from 100 mg to 150 mg, from 150 mg to 200 mg, from 200 mg to 250 mg, from 250 mg to 300 mg, from 300 mg to 350 mg, from 350 mg to 400 mg, from 400 mg to 450 mg, from 450 mg to 500 mg, from 500 mg to 600 mg, from 600 mg to 700 mg, from 700 mg to 800 mg, from 800 mg to 900 mg, from 900 mg to 1 g, from 1 mg to 50 mg, from 20 mg to 40 mg, or from 1 mg to 500 mg of the IL-34 antisense oligonucleotide or a pharmaceutical composition comprising the IL-34 antisense oligonucleotide.

Also provided herein is a method of preventing or treating hepatic fibrosis, pulmonary fibrosis, or intestinal fibrosis, where the method comprises administering to a patient in need thereof a pharmaceutical preparation comprising an IL-34 inhibitor, for example, an IL-34 antisense oligonucleotide such as an IL-34 antisense oligonucleotide disclosed herein. Also provided are methods of preventing or treating renal fibrosis, cardiac fibrosis, endomyocardial fibrosis, idiopathic pulmonary fibrosis, myelofibrosis, retroperitoneal fibrosis, and/or nephrogenic systemic fibrosis, where the method comprises administering to a patient in need thereof a pharmaceutical preparation comprising an IL-34 antisense oligonucleotide such as an IL-34 antisense oligonucleotide disclosed herein.

Also described herein are nucleotide-based IL-34 inhibitors that include, but are not limited to, IL-34 small hairpin RNAs (shRNAs), IL-34 small interfering RNAs (siRNAs), and IL-34 morpholino oligomers, IL-34 microRNAs (miRNAs), and compositions that include such compounds. In some embodiments, an IL-34 inhibitor described herein can include the nucleotide sequence of any one of SEQ ID NOs:1-23, or a pharmaceutically acceptable salt thereof. In some embodiments, an IL-34 inhibitor described herein can include the nucleotide sequence of any one of SEQ ID NOs:1-8, or a pharmaceutically acceptable salt thereof. For example, IL-34 inhibitors include IL-34 small hairpin RNAs, IL-34 small interfering RNAs, IL-34 microRNAs, and IL-34 morpholino oligomers that include the nucleotide sequence of any one of SEQ ID NOs:1-23, for example, the nucleotide sequence of any one of SEQ ID NOs:1-8, and compositions that include such compounds, for example, compositions that include a pharmaceutically acceptable excipient.

In particular embodiments described herein, an IL-34 inhibitor is an IL-34 siRNA that includes the nucleotide sequence of any one of SEQ ID NOs:1-23, for example, the nucleotide sequence of any one of SEQ ID NOs:1-8, or a pharmaceutically acceptable salt thereof. For example, an IL-34 siRNA of the invention can be an siRNA that includes the sequence: 5′-CTCACCAAGACCCACAG-3′ (SEQ ID NO:1); 5′-GGCTTTGGGCCGCACCAGCT-3′ (SEQ ID NO:2); 5′-CTTTGGGCCGCACCAGCTTC-3′ (SEQ ID NO:3); 5′-TGGGCCGCACCAGCTTCAGG-3′ (SEQ ID NO:4); 5′-TCCATGACCCGGAAGCAGTT-3′ (SEQ ID NO:5); 5′-TGTTTCATGTACTGAAG-3′ (SEQ ID NO:6); 5′-CTTTGGGCXGCACCAGCTTC-3′ (SEQ ID NO:7), wherein X is 5-methylcytidine; or 5′-TCCATGACCXGGAAGCAGTT-3′ (SEQ ID NO:8), wherein X is 5-methylcytidine, or a pharmaceutically acceptable salt of an IL-34 siRNA of one of SEQ ID NOs:1-8.

Also described herein are pharmaceutically acceptable compositions that include an IL-34 siRNA of any one of SEQ ID NOs:1-23, for example, an siRNA of the nucleotide sequence of any one of SEQ ID NOs:1-8, or a pharmaceutically acceptable salt thereof, and a pharmaceutically acceptable excipient.

In some embodiments, an IL-34 inhibitor describe herein, for example, an IL-34 siRNA described herein, includes one or more modified internucleoside linkages. For example, in some embodiments, the IL-34 inhibitor is an IL-34 siRNA wherein at least one internucleoside linkage of the sequence is a phosphorothioate linkage, a phosphorodithioate linkage, a phosphotriester linkage, an alkylphosphonate linkage, an aminoalkylphosphotriester linkage, an alkylene phosphonate linkage, a phosphinate linkage, a phosphoramidate linkage, and an aminoalkylphosphoramidate linkage, a thiophosphoramidate linkage, thionoalkylphosphonate linkage, a thionoalkylphosphotriester linkage, a thiophosphate linkage, a selenophosphate linkage, or a boranophosphate linkage. In particular embodiments, at least one internucleoside linkage of the IL-34 inhibitor sequence is a phosphorothioate linkage. For example, in some embodiments, 1, 2, 3, 4, 5, 6, 7, 8, 9, 10, 11, 12, 13, 14, 15, 16, 17, 18, 19, 1-5, 1-10, 1-14, 1-15, 1-16, 1-19, 5-10, 5-14, 5-15, 5-19, 10-14, 10-15, or 10-19 internucleoside linkages of the IL-34 inhibitor sequence are phosphorothioate linkages. In some embodiments, all of the internucleoside linkages of the IL-34 inhibitor sequence are phosphorothioate linkages.

The number of nucleotides included in IL-34 inhibitors described herein may vary. For example, in some embodiments, the IL-34 siRNA is from 20-25, 20-30, or 25-30 nucleotides in length. In some embodiments, the IL-34 siRNA is 19, 20, 21, 22, 23, 24, 25, 26, 27, 28, 29, or 30 nucleotides in length. In some embodiments an IL-34 siRNA described herein includes a maximum number of nucleotides. In some embodiments, an IL-34 siRNA is no more than 19, 20, 21, 22, 23, 24, 25, 26, 27, 28, 29, or 30 nucleotides in length.

In embodiments described herein, a nucleoside of an IL-34 inhibitor, for example, an IL-34 siRNA of any one of SEQ ID NOs:1-23, for example, an IL-34 siRNA of the nucleotide sequence of any one of SEQ ID NOs:1-8, can be substituted with a chemically modified nucleoside. For example, in some embodiments, one or more cytidines of an IL-34 siRNA are replaced with 5-methylcytidine. In some embodiments, an IL-34 siRNA can include one or more modified nucleotides, for example, 5-methyl-2′-deoxycytidine. For example, in varying embodiments, an IL-34 siRNA of the present disclosure includes or can include nucleotides including deoxycytidine and/or 5-methyl-2′-deoxycytidine, including, but not limited to, 5-methyl-2′-deoxycytidine 5′-monophosphate and 5-methyl-2′-deoxycytidine-5′-monophosphorothioate. For example, in embodiments described herein, one or more cytidine nucleosides of an IL-34 siRNA of any one of SEQ ID NOs:1-23, or a pharmaceutically acceptable salt thereof, can each be substituted with 5-methylcytidine.

Additionally, in embodiments described herein, IL-34 inhibitors, for example, IL-34 siRNAs described herein, can include chemically modified nucleosides, for example, 2′-O-methyl (“2′-OMe”) ribonucleosides, for example, 2′-O-methylcytidine, 2′-O-methylguanosine, 2′-O-methylthymidine, 2′-O-methyluridine, and/or 2′-O-methyladenosine. IL-34 inhibitors described herein, can also include one or more chemically modified bases, including a 5-methylpyrimidine, for example, 5-methylcytosine, and/or a 5-methylpurine, for example, 5-methylguanine. IL-34 inhibitors described herein, can also include any of the following chemically modified nucleosides: 5-methyl-2′-O-methylcytidine, 5-methyl-2′-O-methylthymidine, 5-methylcytidine, 5-methyluridine, and/or 5-methyl-2′-deoxycytidine. Additionally, IL-34 inhibitors described herein can include one or more 2′-O-(2-methoxyethyl) (“2′-MOE”) nucleosides, 2′-deoxy-2′-fluoro nucleosides, 2′-fluoro-β-D-arabinonucleosides, bridged nucleic acids, LNA nucleotides, constrained ethyl′ (cET) nucleic acids, tricyclo-DNAs (tcDNA), 2′-0,4′-C-ethylene linked nucleic acids (ENA), and/or peptide nucleic acids (PNA). Embodiments described herein include IL-34 inhibitors of any of SEQ ID NOs:1-23, or a pharmaceutically acceptable salt thereof, that include one or more of any of the aforementioned chemically modified nucleosides. In particular embodiments, an IL-34 siRNA, or a pharmaceutically acceptable salt thereof, includes the sequence of any of SEQ ID NOs:1-23, that is modified to include one or more of any of the aforementioned chemically modified nucleosides.

In some embodiments, the disclosure provides a method of treating an inflammatory disease, a method of inhibiting inflammatory cytokine production in cells of a patient suffering from an inflammatory disease, a method of reducing or inhibiting an IL-34 mediated inflammatory response in one or more cells of a patient suffering from an inflammatory disease, a method of treating an inflammatory disease associated with altered IL-34 expression in a patient in need thereof, a method of inhibiting IL-34-mediated M-CSFR-1 signaling in one or more cells of a patient suffering from an inflammatory disease, a method for preventing or treating fibrosis, a method of preventing or treating intestinal fibrosis, and/or a method of reducing or eliminating a fibrotic stricture in a patient suffering from an inflammatory disease, where the method includes administering an IL-34 inhibitor described herein, for example, an IL-34 siRNA. The aforementioned methods can include a step of administering to a patient in need thereof an effective amount of an IL-34 inhibitor (for example, an IL-34 small hairpin RNA, an IL-34 small interfering RNA, an IL-34 microRNA, or an IL-34 morpholino oligomer of SEQ ID NOs:1-23, for example, any one of SEQ ID NOs:1-8, or a composition that includes such an IL-34 inhibitor), or a pharmaceutically acceptable salt thereof. For example, the aforementioned methods can include a step of administering to a patient in need thereof an effective amount of an IL-34 siRNA of any of SEQ ID NOs:1-23, for example, any of SEQ ID NOs:1-8, or a composition that includes such an IL-34 siRNA, or a pharmaceutically acceptable salt thereof. In some embodiments, the inflammatory disease is an inflammatory bowel disease, for example, inflammatory Crohn's disease or fibrostricturing Crohn's disease.

Also disclosed herein is a method of inhibiting IL-34 expression in a cell of a subject or inhibiting expression of one or more collagens in a cell of a subject, where the method includes administering an IL-34 inhibitor, for example, an IL-34 siRNA described herein. For example, the aforementioned methods can include a step of administering to the subject a pharmaceutically effective amount of a pharmaceutical preparation that includes an IL-34 inhibitor (for example, an IL-34 small hairpin RNA, an IL-34 small interfering RNA, an IL-34 microRNA, or an IL-34 morpholino oligomer of SEQ ID NOs:1-23, for example, any one of SEQ ID NOs:1-8, or a composition that includes such an IL-34 inhibitor), or a pharmaceutically acceptable salt thereof. For example, the aforementioned methods can include a step of administering to the subject a pharmaceutically effective amount of a pharmaceutical preparation that includes an IL-34 siRNA of any of SEQ ID NOs:1-23, for example, any one of SEQ ID NOs:1-8, or a composition that includes such an IL-34 siRNA, or a pharmaceutically acceptable salt thereof. In some embodiments of the aforementioned methods, the subject is in need of treatment of inflammatory Crohn's disease or fibrostricturing Crohn's disease.

Also described herein are methods of treatment wherein an IL-34 siRNA is administered to a patient in need thereof. For example, described herein are methods of treating an inflammatory disease, comprising administering to a patient in need thereof an effective amount of an IL-34 siRNA of any one of SEQ ID NOs:1-8. Also described herein is a method of inhibiting inflammatory cytokine production in cells of a patient suffering from an inflammatory disease, comprising administering an effective amount of an IL-34 siRNA of any one of SEQ ID NOs:1-8.

Also described herein is a method of reducing or inhibiting an IL-34 mediated inflammatory response in a cell or cells of a patient suffering from an inflammatory disease, comprising administering an effective amount of an IL-34 siRNA of any one of SEQ ID NOs:1-8. Also described herein, is a method of treating an inflammatory disease associated with altered IL-34 expression in a patient in need thereof, the method comprising administering an effective amount of an IL-34 siRNA. For example, described herein is a method of treating an inflammatory disease associated with increased IL-34 expression in a patient in need thereof, where the method includes administering an effective amount of an IL-34 siRNA of any one of SEQ ID NOs:1-8, or a pharmaceutically acceptable salt thereof. Increased IL-34 expression may be relative to a control level of IL-34 expression, for example, a mean or median level of IL-34 measured in a healthy control patient or a cohort of healthy control patients, or a level of IL-34 measured in the patient prior to onset or detection of the disease associated with increased IL-34 expression.

Also described herein is a method of treating an inflammatory disease associated with altered IL-34 expression in a patient in need thereof, comprising administering an effective amount of an IL-34 siRNA of any one of SEQ ID NOs:1-8, or a pharmaceutically acceptable salt thereof.

Also described herein is a method of inhibiting IL-34-mediated M-CSFR-1 signaling in cells of a patient suffering from an inflammatory disease, comprising administering an effective amount of an IL-34 siRNA of any one of SEQ ID NOs:1-8, or a pharmaceutically acceptable salt thereof.

Also described herein is a method of reducing or eliminating a fibrotic stricture in a patient suffering from an inflammatory disease, comprising administering an effective amount of an IL-34 siRNA of any one of SEQ ID NOs:1-8, or a pharmaceutically acceptable salt thereof. In some embodiments, the fibrotic stricture is located in the intestine, for example, the large intestine or the small intestine. In particular embodiments, the fibrotic stricture is located in the large intestine. In some embodiments, the fibrotic stricture is localized to one or more portions of the large and/or small intestine, for example, the cecum, the ileum, the ascending colon, the transverse colon, the descending colon, the sigmoid colon, the rectum, the anus, the duodenum, and/or the jejunum.

In some embodiments, the IL-34 inhibitor, for example, the IL-34 siRNA is administered topically, parenterally, orally, pulmonarily, intratracheally, intranasally, transdermally, or intraduodenally. In a particular embodiment, the IL-siRNA, or a pharmaceutically acceptable salt thereof, is administered orally.

Also described herein is a pharmaceutically acceptable composition comprising an IL-34 inhibitor described herein, for example, an IL-34 siRNA of any one of SEQ ID NOs:1-8, or a pharmaceutically acceptable salt thereof, and a pharmaceutically acceptable excipient. In some embodiments, the pharmaceutical composition is suitable for topical, parenteral, oral, pulmonary, intratracheal, intranasal, transdermal, or intraduodenal administration.

Also described herein is an IL-34 inhibitor, for example, an IL-34 siRNA, for use as a medicament. For example, an IL-34 siRNA of any one of SEQ ID NOs:1-8 can be for use as a medicament.

Also described herein is use of an IL-34 inhibitor, for example, an IL-34 siRNA of any one of SEQ ID NOs:1-8, in the manufacture of a medicament for the treatment of an inflammatory disease. Also described herein is an IL-34 inhibitor, for example, an IL-34 siRNA of any one of SEQ ID NOs:1-8, for use in the treatment of an inflammatory disease. In some embodiments, the inflammatory disease is an inflammatory bowel disease, rheumatoid arthritis, psoriasis, osteoarthritis, diabetes (Type I and II), tissue or organ rejection, multiple sclerosis, periodontal inflammation, periodontitis, pigmented villonodular synovitis, hepatitis, sinusitis, colon cancer, colorectal cancer, colitis-associated colon cancer, sporadic colorectal cancer, coronary artery disease, Sjogren's syndrome (SS), obesity, chronic inflammation, pulmonary sarcoidosis, skin lesions, a CNS inflammatory disease, or an autoimmune disease. Also described herein is use of an IL-34 antisense oligonucleotide in the manufacture of a medicament for the treatment of asthma, chronic obstructive pulmonary disease (COPD), or idiopathic pulmonary fibrosis (IPF). In some embodiments, the inflammatory disease is an inflammatory bowel disease. In some embodiments, the inflammatory bowel disease is Crohn's disease, inflammatory Crohn's disease, fibrostricturing Crohn's disease, gastroduodenal Crohn's disease, Crohn's (granulomatous) colitis, ulcerative colitis, collagenous colitis, lymphocytic colitis, ischaemic colitis, diversion colitis, Behçet's disease, microscopic colitis, ulcerative proctitis, proctosigmoiditis, jejunoileitis, left-sided colitis, pancolitis, ileocolitis, ileitis, or indeterminate colitis. In some embodiments, the inflammatory bowel disease is inflammatory Crohn's disease or fibrostricturing Crohn's disease.

Also described herein is a method of inhibiting IL-34 expression in a cell of a subject, comprising administering to the subject a pharmaceutically effective amount of a pharmaceutical preparation that includes an IL-34 inhibitor, or a pharmaceutically acceptable salt thereof, for example, an IL-34 siRNA of any one of SEQ ID NOs:1-8. Also described herein is a method of inhibiting expression of one or more collagens in a cell of a subject, comprising administering to the subject a pharmaceutically effective amount of a pharmaceutical preparation comprising an IL-34 inhibitor, or a pharmaceutically acceptable salt thereof, for example, an IL-34 siRNA of any one of SEQ ID NOs:1-8. In some embodiments of the aforementioned methods, the cell is an intestinal cell, for example, an intestinal stromal cell, an intestinal epithelial cell, an intestinal stem cell, a secretory cell, an enterocyte, a Goblet cell, an enteroendocrine cell, a Paneth cell, a transit amplifying cell, a microfold cell, a cup cell, or a tuft cell. In some embodiments, the cell forms part of an intestinal fibrostricture. In the aforementioned embodiments, subject can be a subject in need of treatment of an inflammatory disease or fibrosis, for example, inflammatory Crohn's disease or fibrostricturing Crohn's disease. In some embodiments, methods described herein can be used to inhibit expression of one or more collagens in a cell of a subject. In certain embodiments, the pharmaceutical preparation can be administered orally. In certain embodiments, the subject is a human.

In some embodiments, the invention can be a method of treating or preventing fibrosis or preventing collagen deposition in a patient, where the method comprises administering to the patient an effective amount of a specific inhibitor of IL-34, for example, an IL-34 siRNA of any one of SEQ ID NOs:1-8, or a pharmaceutically acceptable salt thereof.

Also described herein is use of an IL-34 inhibitor, for example, an IL-34 siRNA, in the manufacture of a medicament for the treatment of fibrosis. In some embodiments, the fibrosis is intestinal fibrosis. In some embodiments, the fibrosis is pulmonary fibrosis. In other embodiments, the fibrosis is renal fibrosis, cardiac fibrosis, endomyocardial fibrosis, myelofibrosis, retroperitoneal fibrosis, or nephrogenic systemic fibrosis.

Also described herein is an IL-34 inhibitor, for example, an IL-34 siRNA, for use in the treatment of fibrosis. In some embodiments, the fibrosis is intestinal fibrosis. In some embodiments, the fibrosis is pulmonary fibrosis. In other embodiments, the fibrosis is renal fibrosis, cardiac fibrosis, endomyocardial fibrosis, myelofibrosis, retroperitoneal fibrosis, or nephrogenic systemic fibrosis.

In various embodiments, an IL-34 inhibitor, for example, an IL-34 siRNA, can be administered by one or several routes, including orally, topically, parenterally, e.g., by subcutaneous injection, by inhalation spray, or rectally. In a preferred embodiment, the IL-34 inhibitor can be administered orally to the patient having fibrosis, for example, intestinal fibrosis or pulmonary fibrosis.

The invention can provide methods that include administration of a IL-34 inhibitor capable of targeting IL-34 RNA for degradation, interfering with RNA splicing, or preventing IL-34 gene expression or protein translation. IL-34 inhibitors of the invention, including IL-34 siRNAs of the invention, can target various regions of mouse and/or human IL-34 mRNA for binding.

In some embodiments of the invention, a method of treating fibrosis, for example, intestinal fibrosis, preventing intestinal fibrosis or pulmonary fibrosis, or preventing collagen deposition in a patient includes administering a pharmaceutical composition, for example, a pharmaceutical composition comprising a specific inhibitor of IL-34, for example, an IL-34 siRNA, and a pharmaceutically acceptable excipient. In some embodiments, the pharmaceutical composition is administered parenterally. In some embodiments, the pharmaceutical composition is administered orally. In some embodiments, the pharmaceutical composition includes an enteric coating, for example, an enteric coating comprising an ethylacrylate-methacrylic acid copolymer.

In embodiments of the invention, methods of treating and/or preventing an inflammatory disease (for example, inflammatory Crohn's disease or fibrostricturing Crohn's disease), treating, reducing, and/or eliminating fibrotic strictures in a patient (for example, in a patient suffering from fibrostricturing Crohn's disease), treating fibrosis (for example, intestinal fibrosis or pulmonary fibrosis), preventing fibrosis (for example, intestinal fibrosis or pulmonary fibrosis), or treating, ameliorating, or preventing collagen deposition in a patient include administering varying amounts of an IL-34 inhibitor, for example an IL-34 siRNA, or a pharmaceutically acceptable salt thereof, or a pharmaceutical composition comprising an IL-34 inhibitor, for example, an IL-34 siRNA of any one of SEQ ID NOs:1-8, or a pharmaceutically acceptable salt thereof. In some embodiments, methods of the invention include administering at least 1 μg, at least 5 μg, at least 10 μg, at least, 20 μg, at least, 30 μg, at least, 40 μg, at least, 50 μg, at least, 60 μg, at least, 70 μg, at least, 80 μg, at least, 90 μg or at least 100 μg of the IL-34 inhibitor. In some embodiments, methods of the invention include administering from 35 mg to 500 mg, from 1 mg to 10 mg, from 10 mg to 20 mg, from 20 mg to 30 mg, from 30 mg to 40 mg, from 40 mg to 50 mg, from 50 mg to 60 mg, form 60 mg to 70 mg, from 70 mg to 80 mg, from 80 mg to 90 mg, from 90 mg to 100 mg, from 100 mg to 150 mg, from 150 mg to 200 mg, from 200 mg to 250 mg, from 250 mg to 300 mg, from 300 mg to 350 mg, from 350 mg to 400 mg, from 400 mg to 450 mg, from 450 mg to 500 mg, from 500 mg to 600 mg, from 600 mg to 700 mg, from 700 mg to 800 mg, from 800 mg to 900 mg, from 900 mg to 1 g, from 1 mg to 50 mg, from 20 mg to 40 mg, or from 1 mg to 500 mg of the IL-34 inhibitor or a pharmaceutical composition comprising the IL-34 inhibitor.

Also provided herein is a method of preventing or treating hepatic fibrosis, pulmonary fibrosis, or intestinal fibrosis, where the method comprises administering to a patient in need thereof a pharmaceutical preparation comprising an IL-34 inhibitor, for example, an IL-34 siRNA disclosed herein. Also provided are methods of preventing or treating renal fibrosis, cardiac fibrosis, endomyocardial fibrosis, idiopathic pulmonary fibrosis, myelofibrosis, retroperitoneal fibrosis, and/or nephrogenic systemic fibrosis, where the method comprises administering to a patient in need thereof a pharmaceutical preparation comprising an IL-34 inhibitor such as an IL-34 siRNA disclosed herein.

### BRIEF DESCRIPTION OF THE DRAWINGS

FIG. 1 is a graph showing normalized IL-34 mRNA expression levels in DLD-1, HT-29, HCT-116, and NCM-460 cells.

FIG. 2A is a graph showing normalized IL-34 mRNA expression in HT-29 cells transfected with a scrambled negative control oligonucleotide (Src AS) or an IL-34 antisense oligonucleotide of SEQ ID NO:1, 2, 3, or 4 (AS34New1, AS34New2, AS34New3, AS34New4, respectively) for 24 hours.

FIG. 2B shows Western blots cell extracts from HT-29 cells transfected with SrcAS control or IL-34 antisense oligonucleotides AS34New1 or AS34New3. Western blots were probed for IL-34 (top) or (β-actin (bottom).

FIG. 2C shows Western blots of extracts from RAW 264.7 cells transfected with SrcAS control or IL-34 antisense oligonucleotides AS34New1, AS34New3, or AS34New4. Western blots were probed for IL-34 (top) or β-actin (bottom).

FIG. 3A shows Western blots of extracts from cultured fibrostricturing Crohn's disease (FS CD) fibroblasts transfected with SrcAS control or IL-34 antisense oligonucleotides AS34New1 or AS34New3. Western blots were probed for IL-34 (top) or β-actin (bottom).

FIG. 3B shows Western blots of extracts from cancer-associated fibroblasts transfected with SrcAS control or the IL-34 antisense oligonucleotide AS34New3. Western blots were probed for IL-34 (top) or β-actin (bottom).

FIG. 4A shows Western blots of extracts from HT-29 cells transfected with one of two control scrambled antisense oligonucleotide (ControlASNew-1-PS or ControlASNew-3-PS) or an IL-34 antisense oligonucleotide of SEQ ID NO:1, 3, or 7 (New1-PS, New-3-PS, and New-3-PS-MEC, respectively). Western blots were probed for IL-34 (top) or β-actin (bottom).

FIG. 4B shows Western blots of extracts from RAW 264.7 cells transfected with one of two control scrambled antisense oligonucleotide (ControlASNew-1-PS or ControlASNew-3-PS) or an IL-34 antisense oligonucleotide of SEQ ID NO:1, 3, or 7 (New1-PS, New-3-PS, and New-3-PS-MEC, respectively). Western blots were probed for IL-34 (top) or β-actin (bottom).

FIG. 4C shows Western blots of extracts from MC-38 cells transfected with one of two control scrambled antisense oligonucleotide (ControlASNew-1-PS or ControlASNew-3-PS) or an IL-34 antisense oligonucleotide of SEQ ID NO:1, 3, or 7 (New1-PS, New-3-PS, and New-3-PS-MEC, respectively). Western blots were probed for IL-34 (top) or β-actin (bottom).

FIG. 5A is a graph showing normalized M-CSFR-1 mRNA expression in ileal control specimens from unaffected areas of ileum of patients undergoing surgery for colon cancer (Ileal CTR) and ileal inflammatory CD (I CD) and fibrostricturing Crohn's disease (FS CD) specimens from CD patients, as evaluated by RT-PCR.

FIG. 5B shows Western blots of extracts from Ileal CTR, I CD, and FS CD tissue samples. Western blots were probed for M-CSFR-1 (top) or β-actin (bottom).

FIG. 5C shows quantitative analysis of relative M-CSFR-1 signal in Western blots of extracts from Ileal CTR, I CD, and FS CD tissue samples. M-CSFR-1 signal was normalized relative to β-actin signal.

FIG. 5D shows immunohistochemical detection of M-CSFR-1 in ileal sections from patients undergoing surgery for colon cancer (Ileal CTR).

FIG. 5D shows immunohistochemical detection of M-CSFR-1 in fibrostricturing Crohn's disease specimens from ileal sections of CD patients (FS CD).

FIG. 5F shows M-CSFR-1 antibody staining of ileal sections with isotype control (Isotype).

FIG. 5G is a graph showing normalized M-CSFR-1 mRNA expression in ileal control specimens (Ileal CTR) and fibrostricturing Crohn's disease (FS CD), as evaluated by RT-PCR.

FIG. 6A is a bar graph showing normalized collagen type I alpha 1 chain (COL1A1) mRNA expression in cultured intestinal fibroblasts either left unstimulated (Unst) or stimulated with recombinant human IL-34 (IL-34), as evaluated by RT-PCR.

FIG. 6B is a bar graph showing normalized collagen type III alpha 1 chain (COL3A1) mRNA expression in cultured intestinal fibroblasts either left unstimulated (Unst) or stimulated with recombinant human IL-34 (IL-34), as evaluated by RT-PCR.

FIG. 6C shows Western blots of extracts from cultured intestinal fibroblasts either left unstimulated (Unst) or stimulated with recombinant human IL-34 (IL-34). Western blots were probed for COL1A1 (top panel), COL3A1 (middle panel), or β-actin (bottom).

FIG. 6D shows graphs of normalized COL1A1 protein expression in cultured intestinal fibroblasts either left unstimulated (Unst) or stimulated with recombinant human IL-34 (IL-34), as evaluated by Western blotting.

FIG. 6E shows graphs of normalized COL3A1 protein expression in cultured intestinal fibroblasts either left unstimulated (Unst) or stimulated with recombinant human IL-34 (IL-34), as evaluated by Western blotting.

FIG. 6F shows total collagen content in supernatants from cultured intestinal fibroblasts either left unstimulated (Unst) or stimulated with recombinant human IL-34 (IL-34).

FIG. 7A shows Western blots of extracts from serum-starved intestinal fibroblasts that were either left unstimulated (Unst) or stimulated with recombinant human IL-34, TNF-α, or IL-6. Blots were probed for total (p38) and phosphorylated p38 (p-p38) mitogen-activated protein (MAP) kinase.

FIG. 7B shows Western blots of extracts from fibroblasts pre-treated with either dimethylsulfoxide control (DMSO) or the p38 inhibitor SB202190 (SB202190) and left unstimulated, or pre-treated with DMSO or SB202190 followed by stimulation with recombinant human IL-34 (IL-34). Blots were probed for COL1A1 (top panel), COL3A1 (middle panel), or β-actin (bottom).

FIG. 7C shows quantification of COL1A1 signal in Western blots of extracts from fibroblasts pre-treated with either DMSO or SB202190 and left unstimulated, or pre-treated with DMSO or SB202190 followed by stimulation with recombinant human IL-34. COL1A1 signal was normalized to β-actin signal.

FIG. 7D shows quantification of COL3A1 signal in Western blots of extracts from fibroblasts pre-treated with either DMSO or SB202190 and left unstimulated, or pre-treated with DMSO or SB202190 followed by stimulation with recombinant human IL-34. COL3A1 signal was normalized to β-actin signal.

FIG. 7E shows total collagen content in supernatants from fibroblasts pre-treated with either DMSO or SB202190 and left unstimulated, or pre-treated with DMSO or SB202190 followed by stimulation with recombinant human IL-34.

FIG. 8A is a bar graph showing normalized IL-34 expression in intestinal specimens taken from ileal control specimens (Ileal CTR), or inflammatory (I CD) or fibrostricturing Crohn's disease (FS CD) intestinal tissue samples, as evaluated by RT-PCR.

FIG. 8B shows Western blots of extracts from paired intestinal mucosal samples from ileal control (Ileal CTR) or inflammatory (I CD) or fibrostricturing Crohn's disease (FS CD) patients. Blots were probed for IL-34 (top panel) or β-actin (bottom panel).

FIG. 8C shows quantification of IL-34 signal in Western blots of extracts from Ileal CTR, I CD, and FS CD samples.

FIG. 8D shows immunohistochemical staining of IL-34 in ileal control tissue (Ileal CTR).

FIG. 8E shows immunohistochemical staining of IL-34 in fibrostricturing Crohn's disease intestinal tissue (FS CD).

FIG. 8F shows staining of intestinal tissue with isotype control (Isotype).

FIG. 8G is a graph showing relative IL-34 mRNA expression in fibroblasts isolated from ileal samples of control (IlealCTR) and fibrostricturing Crohn's disease patient (FS CD) tissue.

FIG. 8H shows Western blots of extracts from fibroblasts isolated from ileal samples of control patients (Ileal CTR) and fibrostricturing Crohn's disease patients (FS CD) and probed for IL-34 (top) and β-actin (bottom) signal.

FIG. 9A shows Western blots of extracts from cultured fibroblasts from fibrostricturing Crohn's disease patients (FS CD) transfected with either an IL-34 antisense oligonucleotide of SEQ ID NO:3 (IL-34AS) or a complementary sense oligonucleotide (NCAS). Blots were probed for IL-34, COL1A1, COL3A1, and β-actin signal.

FIG. 9B shows quantification of IL-34 signal in Western blots of extracts from cultured and transfected FS CD fibroblasts.

FIG. 9C shows quantification of COL1A1 signal in Western blots of extracts from cultured and transfected FS CD fibroblasts.

FIG. 9D shows quantification of COL3A1 signal in Western blots of extracts from cultured and transfected FS CD fibroblasts.

FIG. 9E shows total collagen content in supernatants from cultures of FS CD fibroblasts transfected with an IL-34 antisense oligonucleotide of SEQ ID NO:3 (IL-34AS) or a complementary sense oligonucleotide (NCAS).

FIG. 9F is a graph showing the percent of cell death in cultured FS CD fibroblasts transfected with an IL-34 antisense oligonucleotide of SEQ ID NO:3 (IL-34AS) or a complementary sense oligonucleotide (NCAS).

FIG. 9G shows fluorescence-activated cell sorting (FACS) plots of cultured FS CD fibroblasts transfected with NCAS. Cells were stained for Annexin V and fluorescein isothiocyanate (Annexin V FITC-A; y-axis) and propidium iodide (Propidium Iodide-A; x-axis).

FIG. 9H shows fluorescence-activated cell sorting (FACS) plots of cultured FS CD fibroblasts transfected with IL-34AS. Cells were stained for Annexin V and fluorescein isothiocyanate (Annexin V FITC-A; y-axis) and propidium iodide (Propidium Iodide-A; x-axis).

### DETAILED DESCRIPTION

### IL-34 Inhibitors

IL-34 levels (e.g., IL-34 mRNA or protein levels) and/or activity (e.g., biological activity, for example, IL-34 receptor stimulation) can be decreased using compounds or compositions that target IL-34 gene or an IL-34 gene product (for example, an IL-34 mRNA).

IL-34 inhibitors can be, but are not limited to, nucleotide-based inhibitors of IL-34 (for example, IL-34 small hairpin RNAs (shRNAs), IL-34 microRNAs (miRNAs), IL-34 small interfering RNAs (siRNAs), and IL-34 antisense oligonucleotides, including IL-34 antisense oligonucleotides that include LNA nucleotides, peptide nucleic acids (PNAs), and morpholino oligomers), and compositions that include such compounds.

### IL-34 Antisense Therapeutics

Antisense therapeutics are a class of nucleic acid-based compounds that can be used to inhibit gene expression. Antisense therapeutics may be single- or double-stranded deoxyribonucleic acid (DNA)-based, ribonucleic acid (RNA)-based, mixed DNA and RNA-based, or DNA/RNA chemical analogue compounds. In general, antisense therapeutics are designed to include a nucleotide sequence that is complementary or nearly complementary to an mRNA or pre-mRNA sequence transcribed from a given gene in order to promote binding between the antisense therapeutic and the pre-mRNA or mRNA. Without being bound by theory, it is believed that in most instances antisense therapeutics act by binding to an mRNA or pre-mRNA, thereby inhibiting protein translation, altering pre-mRNA splicing into mature mRNA, and/or causing destruction of mRNA. In most instances, the antisense therapeutic nucleotide sequence is complementary to a portion of a targeted gene's or mRNA's sense sequence. IL-34 antisense therapeutics described herein are oligonucleotide-based compounds that include an oligonucleotide sequence complementary to a IL-34 gene sense, IL-34 pre-mRNA sense, and/or IL-34 mRNA sense sequence, or a portion thereof. IL-34 antisense therapeutics described herein can also be nucleotide chemical analog-based compounds capable of binding to a IL-34 gene sense, IL-34 pre-mRNA sense, and/or IL-34 mRNA sense sequence, or a portion thereof. IL-34 antisense therapeutics include IL-34 antisense oligonucleotides, IL-34 shRNAs, IL-34 siRNAs, IL-34 PNAs, IL-34 LNAs, and IL-34 morpholino oligomers.

Antisense oligonucleotides (AONs) are short oligonucleotide-based sequences that include an oligonucleotide sequence complementary to a target RNA sequence. AONs are typically between 8 to 50 nucleotides in length, for example, 20 nucleotides in length.

AONs described herein may include chemically modified nucleosides. As used herein, “chemically modified nucleosides” means any nucleoside other than adenosine, cytidine, thymidine, guanosine, or uridine. For example, in some embodiments, a chemically modified nucleoside may include, but is not limited to: a locked nucleic acid (LNA), e.g., LNA cytidine, LNA thymidine, LNA adenosine, or LNA guanosine; a nucleoside having a stabilized terminal 5′-phosphate or phosphatase-resistant analogue of 5′-phosphate, e.g., 5′-methyl phosphonate, 5′-methylenephosphonate, a 5′-methylenephosphonate analog, 5′-E-vinyl phosphonate (5′-E-VP), 5′-phosphorothioate, and a 5′-C-methyl analog; a 2′-O-methyl ribonucleoside, e.g., 2′-O-methylcytidine, 2′-O-methylguanosine, 2′-O-methyluridine, 2′-O-methylthymidine, and 2′-O-methyladenosine; a 5-methylpyrimidine, e.g., 5-methylcytosine; a 5-methylpurine, e.g., 5-methylguanine; 5-methyl-2′-O-methylcytidine; 5-methyl-2′-O-methylthymidine; 5-methylcytidine; 5-methyluridine; or 5-methyl 2′-deoxycytidine.

AONs described herein may include at least one modified internucleoside linkage. As used herein, “internucleoside linkage” means the connection between the 3′ position of a nucleoside and the 5′ position of an adjacent nucleoside. A “modified internucleoside linkage” refers to a connection between the 3′ position of a nucleoside and the 5′ position of an adjacent nucleoside that is not natural. For example, in some embodiments, a modified internucleoside linkage may include, but is not limited to: a phosphorothioate linkage; a phosphorodithioate linkage, a phosphotriester linkage, an alkylphosphonate linkage, an aminoalkylphosphotriester linkage, an alkylene phosphonate linkage, a phosphinate linkage, a phosphoramidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, or a boranophosphate linkage

In some embodiments, AONs described herein may include at least one chemically modified nucleoside and at least one modified internucleoside linkage. In some embodiments, AONs described herein may include at least one chemically modified nucleoside or at least one modified internucleoside linkage.

IL-34 AONs described herein include oligonucleotide sequences that are complementary to IL-34 RNA sequences.

Peptide nucleic acids (PNAs) are short, artificially synthesized polymers with a structure that mimics DNA or RNA. PNAs include a backbone composed of repeating N-(2-aminoethyl)-glycine units linked by peptide bonds. IL-34 PNAs described herein can be used as antisense therapeutics that bind to IL-34 RNA sequences with high specificity and inhibit IL-34 gene expression.

Locked nucleic acids (LNAs) are oligonucleotide sequences that include one or more modified RNA nucleotides in which the ribose moiety is modified with an extra bridge connecting the 2′ oxygen and 4′ carbon. LNAs are believed to have higher Tm's than analogous oligonucleotide sequences. IL-34 LNAs described herein can be used as antisense therapeutics that bind to IL-34 RNA sequences with high specificity and inhibit IL-34 gene expression.

Morpholino oligomers are oligonucleotide compounds that include DNA bases attached to a backbone of methylenemorpholine rings linked through phosphorodiamidate groups. Morpholino oligomers of the present invention can be designed to bind to specific IL-34 RNA sequences of interest (for example, IL-34 mRNA or IL-34 pre-mRNA sequences of interest), thereby preventing gene expression. IL-34 morpholino oligomers described herein can be used as antisense therapeutics that bind to IL-34 mRNA sequences with high specificity and inhibit IL-34 gene expression. IL-34 morpholino oligomers described herein can also be used to bind IL-34 pre-mRNA sequences, altering IL-34 pre-mRNA splicing and IL-34 gene expression.

Small hairpin RNAs (shRNAs) are generally RNA molecules with a hairpin-like structure that can be used to silence gene expression. shRNAs are generally expressed from plasmids encoding the shRNA sequence, and can be expressed from viral vectors to allow lentiviral, adenoviral, or adeno-associated viral expression. Without being bound by theory, it is believed that shRNA inhibits gene expression by taking advantage of RNA interference (RNAi) processes. In brief, the shRNA transcript is processed by Drosha and Dicer, and then loaded onto the RNA-induced silencing complex (RISC), allowing targeting of specific mRNA, and either mRNA degradation or repression of protein translation. IL-34 shRNAs described herein can inhibit gene expression of IL-34.

Small interfering RNAs (siRNAs) are double-stranded RNA molecules of approximately 20-25 base pairs in length (but which can also be, for example, 18-30 base pairs in length) that take advantage of RNAi machinery (e.g., Drosha and RISC) to bind and target mRNA for degradation. siRNAs are not dependent upon plasmids or vectors for expression, and can generally be delivered directly to a target cell, for instance, by transfection. IL-34 siRNAs are double-stranded RNA sequences that include an RNA sequence complementary to a IL-34 mRNA sequence, and which prevent IL-34 protein translation. IL-34 siRNAs described herein include siRNAs that include the nucleotide sequence of any one of SEQ ID NOs:1-23, for example, the nucleotide sequence of any one of SEQ ID NOs:1-8, or a pharmaceutically acceptable salt thereof.

MicroRNAs (miRNAs) are small non-coding RNA molecule containing about 22 nucleotides that function in RNA silencing and post-transcriptional regulation of gene expression. miRNAs include sequences that are complementary to portions of an mRNA sequence. miRNAs are produced from long, single-stranded RNA molecules exhibiting highly specific stem-loop structures. Artificial miRNAs are designed by replacing the mature 21 nucleotide sequence of naturally occurring miRNA sequences with 21 nucleotide sequences from a target, for example, an IL-34 mRNA target.

IL-34 inhibitors described herein can include chemical modifications that promote stabilization of an oligonucleotide's terminal 5′-phosphate and phosphatase-resistant analogs of 5′-phosphate. Chemical modifications that promote oligonucleotide terminal 5′-phosphate stabilization or which are phosphatase-resistant analogs of 5′-phosphate include, but are not limited to, 5′-methyl phosphonate, 5′-methylenephosphonate, 5′-methylenephosphonate analogs, 5′-E-vinyl phosphonate (5′-E-VP), 5′-phosphorothioate, and 5′-C-methyl analogs. Chemical modifications that promote AON terminal 5′-phosphate stabilization and phosphatase-resistant analogues of 5′-phosphate are described in Khvorova and Watts, (2017) “The chemical evolution of oligonucleotide therapies of clinical utility” Nat Biotechnol., 35(3):238-48, the contents of which are incorporated by reference herein.

In some embodiments described herein, IL-34 inhibitors can include chemically modified nucleosides, for example, 2′-O-methyl ribonucleosides, for example, 2′-O-methylcytidine, 2′-O-methylguanosine, 2′-O-methyluridine, 2′-O-methylthymidine, and/or 2′-O-methyladenosine. IL-34 inhibitors described herein can include one or more chemically modified bases, including a 5-methylpyrimidine, for example, 5-methylcytosine, and/or a 5-methylpurine, for example, 5-methylguanine. IL-34 inhibitors described herein can include any of the following chemically modified nucleosides: 5-methyl-2′-O-methylcytidine, 5-methyl-2′-O-methylthymidine, 5-methylcytidine, 5-methyluridine, and/or 5-methyl 2′-deoxycytidine.

2′-OMe nucleotides are found naturally in tRNA and other small RNAs. Incorporation of 2′-OMe nucleotides into oligonucleotide sequences prevents nuclease degradation and increases stability against hydrolysis. Incorporation of 2′-OMe modification in oligonucleotides generally increases the Tm of RNA-RNA duplexes by 1-4° C. per addition.

Introduction of a 2′-MOE group generally increases the Tm of the resulting oligonucleotide by about 1.1° C. and improves the resistance to degradation by nuclease. Additionally, 2′-MOE oligos are often used in gapmer compounds to preserve the RNase H-mediated degradation of target mRNA strands.

IL-34 inhibitors described herein can include a phosphate backbone where one or more of the oligonucleoside linkages is a phosphate linkage. IL-34 inhibitors described herein may include a modified oligonucleotide backbone, where one or more of the nucleoside linkages of the nucleotide sequence is selected from the group consisting of a phosphorothioate linkage, a phosphorodithioate linkage, a phosphotriester linkage, an alkylphosphonate linkage, an aminoalkylphosphotriester linkage, an alkylene phosphonate linkage, a phosphinate linkage, a phosphoramidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage. In some embodiments of IL-34 inhibitors described herein, at least one internucleoside linkage of the nucleotide sequence is a phosphorothioate linkage. For example, in some embodiments of IL-34 inhibitors described herein, one, two, three, or more internucleoside linkages of the nucleotide sequence is a phosphorothioate linkage. In some embodiments of IL-34 inhibitors described herein, all internucleoside linkages of the nucleotide sequence are phosphorothioate linkages. Thus, in some embodiments, all of the nucleotide linkages of a IL-34 inhibitor of any of SEQ ID NOs:1-23 are phosphorothioate linkages. In some embodiments, one or more of the nucleotide linkages of a IL-34 inhibitor of any of SEQ ID NOs:1-23 are phosphorothioate linkages. For example, an IL-34 siRNA described herein, can include 1, 2, 3, 4, 5, 6, 7, 8, 9, 10, 11, 12, 13, 14, 15, 16, 17, 18, 19, or more phosphorothioate linkages. In some embodiments, all of the nucleotide linkages of an IL-34 siRNA of any of SEQ ID NOs:1-8 are phosphorothioate linkages.

### IL-34 Antisense Oligonucleotides

IL-34 antisense oligonucleotides (AONs) described herein are short synthetic oligonucleotide sequences complementary to a IL-34 transcript (for example, a IL-34 mRNA transcript).

IL-34 AONs include a nucleotide sequence that is completely or almost completely complementary to a IL-34 mRNA sequence. In some embodiments, a IL-34 AON can include a non-duplexed oligonucleotide. In some embodiments, a IL-34 AON can include a duplex of two oligonucleotides where the first oligonucleotide includes a nucleotide sequence that is completely or almost completely complementary to a IL-34 mRNA sequence and the second oligonucleotide includes a nucleotide sequence that is complementary to the nucleotide sequence of the first oligonucleotide. AON binding specificity can be assessed via measurement of parameters such as dissociation constant, melting temperature (Tm), or other criteria such as changes in protein or RNA expression levels or other assays that measure IL-34 activity or expression.

An IL-34 AON, such as disclosed herein, may be an oligonucleotide sequence of, for example, 15, 16, 17, 18, 19, 20, 21, 22, 23, 24, or 25 nucleotides in length, or 15-20, 15-25, 15-30, 15-35, 20-25, 20-30, 20-35, 25-30, 25-35, or 30-35 nucleotides in length. In some embodiments, an IL-34 antisense oligonucleotide is constrained by an upper size limit so that the AON is no more than 20, 21, 22, 23, 24, 25, 26, 27, 28, 29, 30, 31, 32, 33, 34, or 35 nucleotides in length.

A IL-34 AON may be an oligonucleotide sequence complementary to a portion of a IL-34 mRNA sequence or a IL-34 gene sequence, for example a mouse or human IL-34 mRNA or gene sequence.

In some embodiments of the disclosure, a IL-34 AON includes a nucleotide sequence that is complementary or nearly complementary to a portion of an IL-34 mRNA transcript variant, or a portion thereof, for example, human IL-34 mRNA sequence of NCBI Reference Sequence NM_001172771.1 (SEQ ID NO:24), NM_001172772.1 (SEQ ID NO:25), or NM_152456.2 (SEQ ID NO:26); or mouse IL-34 mRNA sequence of NCBI Reference Sequence NM_001135100.2 (SEQ ID NO:27) or NM_029646.3 (SEQ ID NO:28).

In embodiments of the invention described herein, a IL-34 AON can target IL-34 mRNAs produced from IL-34 genes of one or more species, for example, mouse and human IL-34 mRNA transcripts. For example, a IL-34 AON can target a IL-34 mRNA of a mammalian IL-34 gene, for example, a human (i.e., Homo sapiens) IL-34 gene or a mouse (i.e., Mus musculus) IL-34 gene. In particular embodiments, the IL-34 AON targets a human IL-34 mRNA. In some embodiments, the IL-34 AON includes a nucleotide sequence that is complementary to a nucleotide sequence of a IL-34 gene or a IL-34 mRNA, or a portion thereof.

IL-34 AONs described herein include a IL-34 AON that includes the nucleotide sequence: 5′-CTCACCAAGACCCACAG-3′ (SEQ ID NO:1); 5′-GGCTTTGGGCCGCACCAGCT-3′ (SEQ ID NO:2); 5′-CTTTGGGCCGCACCAGCTTC-3′ (SEQ ID NO:3); 5′-TGGGCCGCACCAGCTTCAGG-3′ (SEQ ID NO:4); 5′-TCCATGACCCGGAAGCAGTT-3′ (SEQ ID NO:5); 5′-TGTTTCATGTACTGAAG-3′ (SEQ ID NO:6); 5′-CTTTGGGCXGCACCAGCTTC-3′ (SEQ ID NO:7); or 5′-TCCATGACCXGGAAGCAGTT-3′ (SEQ ID NO:8), or a pharmaceutically acceptable salt thereof, wherein X is 5-methylcytidine.

IL-34 AONs described herein, can include chemically modified nucleosides, including modified ribonucleosides and modified deoxyribonucleosides. Chemically modified nucleosides include, but are not limited to, 2′-O-(2-methoxyethyl) modifications, for example, 2′-O-(2-methoxyethyl)guanosine, 2′-O-(2-methoxyethyl)adenosine, 2′-O-(2-methoxyethyl)cytosine, 2′-O-(2-methoxyethyl)uridine, and 2′-O-(2-methoxyethyl)thymidine. In certain embodiments, the disclosure provides mixed modalities of IL-34 inhibitors, e.g., a combination of a IL-34 peptide nucleic acid (PNA) and a IL-34 locked nucleic acid (LNA). Chemically modified nucleosides also include, but are not limited to, locked nucleic acids (LNAs), 2′-O-methyl, 2′-fluoro, and 2′-fluoro-β-D-arabinonucleotide (FANA) modifications. Chemically modified nucleosides that can be included in IL-34 AONs described herein are described in Johannes and Lucchino, (2018) “Current Challenges in Delivery and Cytosolic Translocation of Therapeutic RNAs” Nucleic Acid Ther. 28(3): 178-93; Rettig and Behlke, (2012) “Progress toward in vivo use of siRNAs-II” Mol Ther 20:483-512; and Khvorova and Watts, (2017) “The chemical evolution of oligonucleotide therapies of clinical utility” Nat Biotechnol., 35(3):238-48, the contents of each of which are incorporated by reference herein.

IL-34 AONs described herein can include chemical modifications that promote stabilization of an oligonucleotide's terminal 5′-phosphate and phosphatase-resistant analogs of 5′-phosphate. Chemical modifications that promote oligonucleotide terminal 5′-phosphate stabilization or which are phosphatase-resistant analogs of 5′-phosphate include, but are not limited to, 5′-methyl phosphonate, 5′-methylenephosphonate, 5′-methylenephosphonate analogs, 5′-E-vinyl phosphonate (5′-E-VP), 5′-phosphorothioate, and 5′-C-methyl analogs. Chemical modifications that promote AON terminal 5′-phosphate stabilization and phosphatase-resistant analogues of 5′-phosphate are described in Khvorova and Watts, (2017) “The chemical evolution of oligonucleotide therapies of clinical utility” Nat Biotechnol., 35(3):238-48, the contents of which are incorporated by reference herein.

In some embodiments described herein, IL-34 AONs described herein can include chemically modified nucleosides, including 2′-O-methyl ribonucleosides, for example, 2′-O-methylcytidine, 2′-O-methylguanosine, 2′-O-methyluridine, 2′-O-methylthymidine, and/or 2′-O-methyladenosine. IL-34 AONs described herein can include one or more chemically modified nucleosides, wherein the chemically modified nucleosides comprise bases, including a 5-methylpyrimidine, for example, 5-methylcytosine, and/or a 5-methylpurine, for example, 5-methylguanine. IL-34 AONs described herein can include any of the following chemically modified nucleosides: 5-methyl-2′-O-methylcytidine, 5-methyl-2′-O-methylthymidine, 5-methylcytidine, 5-methyluridine, and/or 5-methyl 2′-deoxycytidine.

In some embodiments, IL-34 AONs described herein include a IL-34 AON that includes the nucleotide sequence: 5′-CTCACCAAGACCCACAG-3′ (SEQ ID NO:1), wherein at least one nucleoside is a chemically modified nucleoside and/or at least one linkage is a modified internucleoside linkage; 5′-GGCTTTGGGCCGCACCAGCT-3′ (SEQ ID NO:2), wherein at least one nucleoside is a chemically modified nucleoside and/or at least one linkage is a modified internucleoside linkage; 5′-CTTTGGGCCGCACCAGCTTC-3′ (SEQ ID NO:3), wherein at least one nucleoside is a chemically modified nucleoside and/or at least one linkage is a modified internucleoside linkage; 5′-TGGGCCGCACCAGCTTCAGG-3′ (SEQ ID NO:4), wherein at least one nucleoside is a chemically modified nucleoside and/or at least one linkage is a modified internucleoside linkage; 5′-TCCATGACCCGGAAGCAGTT-3′ (SEQ ID NO:5), wherein at least one nucleoside is a chemically modified nucleoside and/or at least one linkage is a modified internucleoside linkage; or 5′-TGTTTCATGTACTGAAG-3′ (SEQ ID NO:6), wherein at least one nucleoside is a chemically modified nucleoside and/or at least one linkage is a modified internucleoside linkage, or a pharmaceutically acceptable salt thereof.

In some embodiments, an IL-34 AON comprises the nucleotide sequence of SEQ ID NO:3, wherein at least one nucleoside is a chemically modified nucleoside. For example, in some embodiments, the third cytidine (from the 5′ end) of SEQ ID NO:3 may be substituted with a chemically modified nucleoside. In some embodiments, the third cytidine (from the 5′ end) of SEQ ID NO:3 is substituted for 5-methylcytidine, resulting in an IL-34 AON comprising the nucleotide sequence of SEQ ID NO:7. In some embodiments, a nucleoside other than the third cytidine (from the 5′ end) of SEQ ID NO:3 is substituted with a chemically modified nucleoside, resulting in an IL-34 AON having a nucleotide sequence other than SEQ ID NO:7. In some embodiments, the third cytidine (from the 5′ end) of SEQ ID NO:3 is substituted with a chemically modified nucleoside that is not 5-methylcytidine, resulting in an IL-34 AON having a nucleotide sequence other than SEQ ID NO:7.

In some embodiments, an IL-34 AON comprises the nucleotide sequence of SEQ ID NO:5, wherein at least one nucleoside is a chemically modified nucleoside. For example, in some embodiments, the fifth cytidine (from the 5′ end) of SEQ ID NO:5 may be substituted with a chemically modified nucleoside. In some embodiments the fifth cytidine (from the 5′ end) of SEQ ID NO:5 is substituted for 5-methylcytidine, resulting in an IL-34 AON comprising the nucleotide sequence of SEQ ID NO:8. In some embodiments a nucleoside other than the fifth cytidine (from the 5′ end) of SEQ ID NO:3 is substituted with a chemically modified nucleoside, resulting in an IL-34 AON having a nucleotide sequence other than SEQ ID NO:8. In some embodiments, the fifth cytidine (from the 5′ end) of SEQ ID NO:3 is substituted with a chemically modified nucleoside that is not 5-methylcytidine, resulting in an IL-34 AON having a nucleotide sequence other than SEQ ID NO:8.

IL-34 AONs described herein also include IL-34 AONs that include one or more LNA nucleotides. For example, IL-34 AONs described herein include a IL-34 AON that includes the nucleotide sequence of any of the following:
5′-cttTGGGCXGCACCAGCttc-3′ (SEQ ID NO:9), wherein c is LNA cytidine, t is LNA thymidine, and X is 5-methylcytidine;
5′-ctttGGGCXGCACCAGcttc-3′ (SEQ ID NO:10), wherein c is LNA cytidine, t is LNA thymidine, and X is 5-methylcytidine;
5′-cttTGGGCcgCACCAGCttc-3′ (SEQ ID NO:11), wherein c is LNA cytidine, t is LNA thymidine, and g is LNA guanosine;
5′-cttTGGGCcGCACCAGCttc-3′ (SEQ ID NO:12), wherein c is LNA cytidine and t is LNA thymidine;
5′-ggcXGCACCAGCttc-3′ (SEQ ID NO:13), wherein c is LNA cytidine, t is LNA thymidine, g is LNA guanosine, and X is 5-methylcytidine;
5′-cttTGGGCXGCACcag-3′ (SEQ ID NO:14), wherein c is LNA cytidine, t is LNA thymidine, g is LNA guanosine, a is LNA adenosine, and X is 5-methylcytidine; and 5′-tgaCCXGGAAGCAgtt-3′ (SEQ ID NO:15), wherein a is LNA adenosine, t is LNA thymidine, g is LNA guanosine, and X is 5-methylcytidine,
   or a pharmaceutically acceptable salt thereof.

In some embodiments, an IL-34 AON comprises the nucleotide sequence of SEQ ID NO:3, wherein at least one nucleoside is an LNA. For example, in some embodiments, the first and twentieth nucleosides of SEQ ID NO:3 (relative to the 5′ end) are each independently substituted with LNA cytidine; the second, third, eighteenth, and nineteenth nucleosides of SEQ ID NO:3 are each independently substituted with LNA thymidine; and the ninth nucleoside of SEQ ID NO:3 is substituted with 5-methylcytidine, resulting in an IL-34 AON comprising the nucleotide sequence of SEQ ID NO:9.

In another embodiment, the first, seventeenth, and twentieth nucleosides of SEQ ID NO:3 (relative to the 5′ end) are each independently substituted with LNA cytidine; the second, third, fourth, eighteenth, and nineteenth nucleosides of SEQ ID NO:3 are each independently substituted with LNA thymidine; and the ninth nucleoside of SEQ ID NO:3 is substituted with 5-methylcytidine, resulting in an IL-34 AON comprising the nucleotide sequence of SEQ ID NO:10.

In another embodiment, the first, ninth, and twentieth nucleosides of SEQ ID NO:3 (relative to the 5′ end) are each independently substituted with LNA cytidine; the second, third, eighteenth, and nineteenth nucleosides of SEQ ID NO:3 are each independently substituted with LNA thymidine; and the tenth nucleoside of SEQ ID NO:3 is substituted with LNA guanosine, resulting in an IL-34 AON comprising the nucleotide sequence of SEQ ID NO:11.

In yet another embodiment, the first, ninth, and twentieth nucleosides of SEQ ID NO:3 (relative to the 5′ end) are each independently substituted with LNA cytidine; and the second, third, eighteenth, and nineteenth nucleosides of SEQ ID NO:3 are each independently substituted with LNA thymidine, resulting in an IL-34 AON comprising the nucleotide sequence of SEQ ID NO:12.

In other embodiments, one or more nucleoside(s) in SEQ ID NO:3 may be substituted with LNA nucleotides other than those substituted in any one of SEQ ID NO:9, 10, 11, or 12. In other embodiments, SEQ ID NO:3 may be substituted with LNA nucleotides at one or more positions other than those substituted in any one of SEQ ID NO:9, 10, 11, or 12.

In particular embodiments, an IL-34 AON includes 2′-MOE nucleosides and the sequence of the IL-34 AON is selected from one of the following:
5′-CxTxTxTxGxGGCXGCACCAGxCxTxTxCx-3′ (SEQ ID NO:16), wherein Cx is 2′-O-(2-methoxyethyl)cytidine, Tx is 2′-O-(2-methoxyethyl)thymidine, Gx is 2′-O-(2-methoxyethyl)guanosine, and X is 5-methylcytidine;
5′-TxCxCxAxTxGACCXGGAAGCxAxGxTxTx-3′ (SEQ ID NO:17), wherein Cx is 2′-O-(2-methoxyethyl)cytidine, Tx is 2′-O-(2-methoxyethyl)thymidine, Gx is 2′-O-(2-methoxyethyl)guanosine, Ax is 2′-O-(2-methoxyethyl)adenosine, and X is 5-methylcytidine;
5′-CxTxTxTxGxGxGCXGCACCAxGxCxTxTxCx-3′ (SEQ ID NO:18), wherein Cx is 2′-O-(2-methoxyethyl)cytidine, Tx is 2′-O-(2-methoxyethyl)thymidine, Gx is 2′-O-(2-methoxyethyl)guanosine, Ax is 2′-O-(2-methoxyethyl)adenosine, and X is 5-methylcytidine;
5′-TxCxCxAxTxGxACCXGGAAGxCxAxGxTxTx-3′ (SEQ ID NO:19), wherein Cx is 2′-O-(2-methoxyethyl)cytidine, Tx is 2′-O-(2-methoxyethyl)thymidine, Gx is 2′-O-(2-methoxyethyl)guanosine, Ax is 2′-O-(2-methoxyethyl)adenosine, and X is 5-methylcytidine;
5′-CxTxTxTxGxGxGxCxXGxCxAxCxCxAxGxCxTxTxCx-3′ (SEQ ID NO:20), wherein Cx is 2′-O-(2-methoxyethyl)cytidine, Tx is 2′-O-(2-methoxyethyl)thymidine, Gx is 2′-O-(2-methoxyethyl)guanosine, Ax is 2′-O-(2-methoxyethyl)adenosine, and X is 5-methylcytidine; and
5′-TxCxCxAxTxGxAxCxCxXGxGxAxAxGxCxAxGxTxTx-3′ (SEQ ID NO:21), wherein Cx is 2′-O-(2-methoxyethyl)cytidine, Tx is 2′-O-(2-methoxyethyl)thymidine, Gx is 2′-O-(2-methoxyethyl)guanosine, Ax is 2′-O-(2-methoxyethyl)adenosine, and X is 5-methylcytidine,
   or a pharmaceutically acceptable salt thereof.

For example, in some embodiments, an IL-34 AON comprises the nucleotide sequence of SEQ ID NO:3, wherein at least one nucleoside is a 2′-MOE nucleoside. For example, in some embodiments, the first, seventeenth, and twentieth nucleosides of SEQ ID NO:3 (relative to the 5′ end) are each independently substituted with 2′-O-(2-methoxyethyl)cytidine; the second, third, fourth, eighteenth, and nineteenth nucleosides of SEQ ID NO:3 are each independently substituted with 2′-O-(2-methoxyethyl)thymidine; the fifth and sixteenth nucleosides of SEQ ID NO:3 are each independently substituted with 2′-O-(2-methoxyethyl)guanosine; and the ninth nucleoside of SEQ ID NO:3 is substituted with 5-methylcytidine, resulting in an IL-34 AON comprising the nucleotide sequence of SEQ ID NO:16.

In some embodiments, the first, seventeenth, and twentieth nucleosides of SEQ ID NO:3 (relative to the 5′ end) are each independently substituted with 2′-O-(2-methoxyethyl)cytidine; the second, third, fourth, eighteenth, and nineteenth nucleosides of SEQ ID NO:3 are each independently substituted with 2′-O-(2-methoxyethyl)thymidine; the fifth, sixth and sixteenth nucleosides of SEQ ID NO:3 are each independently substituted with 2′-O-(2-methoxyethyl)guanosine; the fifteenth nucleoside of SEQ ID NO:3 is substituted with 2′-O-(2-methoxyethyl)adenosine; and the ninth nucleoside of SEQ ID NO:3 is substituted with 5-methylcytidine, resulting in an IL-34 AON comprising the nucleotide sequence of SEQ ID NO:18.

In some embodiments, the first, eighth, eleventh, thirteenth, fourteenth, seventeenth, and twentieth nucleosides of SEQ ID NO:3 (relative to the 5′ end) are each independently substituted with 2′-O-(2-methoxyethyl)cytidine; all thymidines of SEQ ID NO:3 are each independently substituted with 2′-O-(2-methoxyethyl)thymidine; all guanosines of SEQ ID NO:3 are each independently substituted with 2′-O-(2-methoxyethyl)guanosine; all adenosines of SEQ ID NO:3 are each independently substituted with 2′-O-(2-methoxyethyl)adenosine; and the ninth nucleoside of SEQ ID NO:3 is substituted with 5-methylcytidine, resulting in an IL-34 AON comprising the nucleotide sequence of SEQ ID NO:20.

In other embodiments, one or more nucleoside(s) in SEQ ID NO:3 may be substituted with 2′-MOE nucleosides other than those substituted in any one of SEQ ID NO:16, 18, or 20. In still other embodiments, SEQ ID NO:3 may be substituted with 2′-MOE nucleosides at one or more positions other than those substituted in any one of SEQ ID NO:16, 18, or 20.

In some embodiments, an IL-34 AON comprises the nucleotide sequence of SEQ ID NO:5, wherein at least one nucleoside is a 2′-MOE nucleoside. For example, in some embodiments, the first, fifth, nineteenth, and twentieth nucleosides of SEQ ID NO:5 (relative to the 5′ end) are each independently substituted with 2′-O-(2-methoxyethyl)thymidine; the second, third, and sixteenth nucleosides of SEQ ID NO:5 are each independently substituted with 2′-O-(2-methoxyethyl)cytidine; the fourth and seventeenth nucleosides of SEQ ID NO:5 are each independently substituted with 2′-O-(2-methoxyethyl)adenosine; the eighteenth nucleoside of SEQ ID NO:5 is substituted with 2′-O-(2-methoxyethyl)guanosine; and the tenth nucleoside of SEQ ID NO:5 is substituted with 5-methylcytidine, resulting in an IL-34 AON comprising the nucleotide sequence of SEQ ID NO:17.

In some embodiments, the first, fifth, nineteenth, and twentieth nucleosides of SEQ ID NO:5 (relative to the 5′ end) are each independently substituted with 2′-O-(2-methoxyethyl)thymidine; the second, third, and sixteenth nucleosides of SEQ ID NO:5 are each independently substituted with 2′-O-(2-methoxyethyl)cytidine; the fourth and seventeenth nucleosides of SEQ ID NO:5 are each independently substituted with 2′-O-(2-methoxyethyl)adenosine; the sixth, fifteenth, and eighteenth nucleosides of SEQ ID NO:5 are each independently substituted with 2′-O-(2-methoxyethyl)guanosine; and the tenth nucleoside of SEQ ID NO:5 is substituted with 5-methylcytidine, resulting in an IL-34 AON comprising the nucleotide sequence of SEQ ID NO:19.

In some embodiments, the second, third, eighth, ninth, and sixteenth nucleosides of SEQ ID NO:5 (relative to the 5′ end) are each independently substituted with 2′-O-(2-methoxyethyl)cytidine; all thymidines of SEQ ID NO:5 are each independently substituted with 2′-O-(2-methoxyethyl)thymidine; all guanosines of SEQ ID NO:5 are each independently substituted with 2′-O-(2-methoxyethyl)guanosine; all adenosines of SEQ ID NO:5 are each independently substituted with 2′-O-(2-methoxyethyl)adenosine; and the tenth nucleoside of SEQ ID NO:5 is substituted with 5-methylcytidine, resulting in an IL-34 AON comprising the nucleotide sequence of SEQ ID NO:21.

In other embodiments, one or more nucleoside(s) in SEQ ID NO:5 may be substituted with 2′-MOE nucleosides other than those substituted in any one of SEQ ID NO:17, 19, or 21. In still other embodiments, SEQ ID NO:5 may be substituted with 2′-MOE nucleosides at one or more positions other than those substituted in any one of SEQ ID NO:17, 19, or 21.

Additionally, in some embodiments, an IL-34 AON includes 2′-OMe nucleosides and the sequence of the IL-34 AON is selected from one of the following:
5′-CyTyTyTyGyGGCXGCACCAGyCyTyTyCy-3′ (SEQ ID NO:22), wherein Cy is 2′-O-methylcytidine, Ty is 2′-O-methylthymidine, Gy is 2′-O-methylguanosine, and X is 5-methylcytidine; and
5′-TyCyCyAyTyGACCXGGAAGCyAyGyTyTy-3′ (SEQ ID NO:23), wherein Cy is 2′-O-methylcytidine, Ty is 2′-O-methylthymidine, Gy is 2′-O-methylguanosine, Ay is 2′-O-methyladenosine, and X is 5-methylcytidine,
   or a pharmaceutically acceptable salt thereof.

For example, in some embodiments, an IL-34 AON comprises the nucleotide sequence of SEQ ID NO:3, wherein at least one nucleoside is a 2′-OMe nucleoside. For example, in some embodiments, the first, seventeenth and twentieth nucleosides of SEQ ID NO:3 (relative to the 5′ end) are each independently substituted with 2′-O-methylcytidine; the second, third, fourth, eighteenth, and nineteenth nucleosides of SEQ ID NO:3 are each independently substituted with 2′-O-methylthymidine; the fifth and sixteenth nucleosides of SEQ ID NO:3 are each independently substituted with 2′-O-methylguanosine; and the ninth nucleoside of SEQ ID NO:3 is substituted with 5-methylcytidine, resulting in an IL-34 AON comprising the nucleotide sequence of SEQ ID NO:22.

In other embodiments, one or more nucleoside(s) in SEQ ID NO:3 may be substituted with 2′-OMe nucleosides other than those substituted in SEQ ID NO:22. In still other embodiments, SEQ ID NO:3 may be substituted with 2′-OMe nucleosides at one or more positions other than those substituted in SEQ ID NO:22.

In some embodiments, an IL-34 AON comprises the nucleotide sequence of SEQ ID NO:5, wherein at least one nucleoside is a 2′-OMe nucleoside. For example, in some embodiments, the first, fifth, nineteenth, and twentieth nucleosides of SEQ ID NO:5 (relative to the 5′ end) are each independently substituted with 2′-O-methylthymidine; the second, third, and sixteenth nucleosides of SEQ ID NO:5 are each independently substituted with 2′-O-methylcytidine; the fourth and seventeenth nucleosides of SEQ ID NO:5 are each independently substituted with 2′-O-methyladenosine; the eighteenth nucleoside of SEQ ID NO:5 is substituted with 2′-O-methylguanosine; and the tenth nucleoside of SEQ ID NO:5 is substituted with 5-methylcytidine, resulting in an IL-34 AON comprising the nucleotide sequence of SEQ ID NO:23.

In other embodiments, one or more nucleoside(s) in SEQ ID NO:5 may be substituted with 2′-OMe nucleosides other than those substituted in SEQ ID NO:23. In still other embodiments, SEQ ID NO:5 may be substituted with 2′-OMe nucleosides at one or more positions other than those substituted in SEQ ID NO:23.

IL-34 AONs described herein can include a phosphate backbone where one or more of the oligonucleoside linkages is a modified internucleoside linkage. For example, in some embodiments, an IL-34 AON described herein may comprise at least one phosphate linkage. IL-34 AONs described herein may include one or more modified internucleoside linkages selected from the group consisting of a phosphorothioate linkage, a phosphorodithioate linkage, a phosphotriester linkage, an alkylphosphonate linkage, an aminoalkylphosphotriester linkage, an alkylene phosphonate linkage, a phosphinate linkage, a phosphoramidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage. In some embodiments of IL-34 AONs described herein, at least one internucleoside linkage of the nucleotide sequence is a phosphorothioate linkage. For example, in some embodiments of IL-34 AONs described herein, one, two, three, or more internucleoside linkages of the nucleotide sequence is a phosphorothioate linkage. In preferred embodiments of IL-34 AONs described herein, all internucleoside linkages of the nucleotide sequence are phosphorothioate linkages. Thus, in some embodiments, all of the nucleotide linkages of a IL-34 AON of any of SEQ ID NOs:1-23 are phosphorothioate linkages. For example, in some embodiments, all of the nucleotide linkages of an IL-34 AON of SEQ ID NO:3 are phosphorothioate linkages. In another example, in some embodiments, all of the nucleotide linkages of an IL-34 AON of SEQ ID NO:7 are phosphorothioate linkages. In some embodiments, one or more of the nucleotide linkages of a IL-34 AON of any of SEQ ID NOs:1-23 are phosphorothioate linkages. For example, in some embodiments, one or more of the nucleotide linkages of an IL-34 AON of SEQ ID NO:3 are phosphorothioate linkages. In another example, in some embodiments, one or more of the nucleotide linkages of an IL-34 AON of SEQ ID NO:7 are phosphorothioate linkages.

It is contemplated that in some embodiments, a disclosed IL-34 AON, or a pharmaceutically acceptable salt thereof, may optionally have at least one modified nucleobase, e.g., 5-methylcytosine, and/or at least one methylphosphonate nucleotide, which is placed, for example, either at only one of the 5′ or 3′ ends or at both 5′ and 3′ ends or along the oligonucleotide sequence.

Contemplated IL-34 AONs may optionally include at least one modified sugar. For example, the sugar moiety of at least one nucleotide constituting the oligonucleotide is a ribose in which the 2′—OH group may be replaced by any one selected from the group consisting of OR, R, R′OR, SH, SR, NH₂, NR₂, N₃, CN, F, Cl, Br, and I (wherein R is an alkyl or aryl and R′ is an alkylene).

In some embodiments described herein, an IL-34 AON is a gapmer compound. Gapmer compounds are oligonucleotide sequences that includes 5′ and 3′ flanking groups of modified nucleotides. These flanking groups of modified nucleotides are thought to protect the internal group of nucleotides from nuclease degradation. The internal group of nucleotides is usually from 6 to 10 nucleotides in length. Each 5′ or 3′ group of flanking nucleotides can be 3, 4, 5, 6, or more nucleotides in length. The 5′ and 3′ group of flanking nucleotides in a gapmer compound can be the same number of nucleotides in length. Flanking groups of modified nucleotides include, for example, 2′-MOE, 2′-OMe, and LNA nucleotides. Gapmer compound sequences can also incorporate modifications including modified internucleoside linkages (for example, phosphorothioate linkages), 2′-MOEs, 2′-OMe's, LNAs, PNAs, 5-methylcytidine, and other chemically modified nucleosides described herein.

Antisense oligonucleotides can be designed such that the targeting portion of the incorporated nucleotide sequence of each antisense oligonucleotide is completely or almost completely complementary to the IL-34 mRNA sequence or the mRNA sequence of an IL-34 interaction partner. Incorporation of such complementary or nearly complementary nucleotide sequences allows one to engineer antisense oligonucleotides with a high degree of specificity for a given target. Specificity can be assessed via measurement of parameters such as dissociation constant, or other criteria such as changes in protein or RNA expression levels or other assays that measure IL-34 activity or expression.

### Autoimmune and Inflammatory Diseases

The disclosure contemplates, in part, treating inflammatory disorders related to IL-34 activities in a patient in need thereof comprising administering a disclosed antisense compound. In some embodiments, provided herein are methods for treatment of an inflammatory disease in a patient in need thereof, comprising administering a disclosed antisense compound. In some embodiments of the disclosure, an effective amount of a disclosed IL-34 inhibitor, for example an IL-34 antisense oligonucleotide, may be administered to a patient in need thereof to treat an inflammatory disease, for example, to inhibit inflammatory cytokine production in cells of a patient suffering from an inflammatory disease, and/or to reduce or inhibit an IL-34 mediated inflammatory response.

“Inflammatory disease” as used herein, refers to a number of acute and chronic inflammatory disorders including but not limited to inflammatory bowel disease, rheumatoid arthritis, psoriasis, osteoarthritis, diabetes (type I and II), tissue or organ rejection, multiple sclerosis, periodontal inflammation (e.g., periodontitis), pigmented villonodular synovitis, hepatitis, sinusitis, colon cancer, coronary artery disease, Sjogren's syndrome (SS), obesity, chronic inflammation, pulmonary sarcoidosis, skin lesions, a CNS inflammatory disease, or an autoimmune disease.

In some embodiments, methods of treating skin lesions associated with lupus in a patient in need thereof are provided comprising administering a disclosed compound. In some embodiments, treating skin lesions associated with lupus comprises at least one effect selected from reducing the number of skin lesions, reducing the rate of formation of skin lesions, and reducing the severity of skin lesions. Methods of treating lupus and/or lupus nephritis in a patient suffering therefrom are provided, that include administering a disclosed antisense compound. In some embodiments, methods of slowing the progression of a kidney condition associated with lupus are provided.

Provided herein are methods of treating, reducing the risk of developing, or delaying the onset of a CNS inflammatory disease in a subject in need thereof comprising administering a disclosed compound. The methods include for example, treating a subject at risk of developing a CNS inflammatory disease; e.g., administering to the subject an effective amount of a disclosed IL-34 inhibitor, for example an IL-34 antisense oligonucleotide. CNS inflammatory disease that can be treated in this manner include multiple sclerosis, experimental autoimmune encephalomyelitis, Alzheimer's disease, amyotrophic lateral sclerosis (ALS), and Parkinson's disease.

Also provided herein are methods of treating, reducing the risk of developing, or delaying the onset of an autoimmune disease in a subject in need thereof comprising administering a disclosed compound, for example, an IL-34 inhibitor, for example an IL-34 antisense oligonucleotide, described herein. The methods include treating a subject with or at risk of developing an autoimmune disease. Autoimmune diseases that can be treated in this manner include rheumatoid arthritis, type I diabetes, asthma, chronic obstructive pulmonary disease (COPD), and idiopathic pulmonary fibrosis.

### Inflammatory Bowel Disease

The present disclosure also provides methods for treatment of an inflammatory bowel disease to a patient in need thereof comprising administering a disclosed compound. “Inflammatory bowel disease,” as used herein, refers to a number of chronic inflammatory diseases including Crohn's disease, inflammatory Crohn's disease, fibrostricturing Crohn's disease, gastroduodenal Crohn's disease, Crohn's (granulomatous) colitis, ulcerative colitis, collagenous colitis, lymphocytic colitis, ischaemic colitis, diversion colitis, Behcet's disease, microscopic colitis, ulcerative proctitis, proctosigmoiditis, jejunoileitis, left-sided colitis, pancolitis, ileocolitis, ileitis, and indeterminate colitis. Crohn's disease and ulcerative colitis are the two most common forms of inflammatory bowel disease. Inflammatory bowel diseases are autoimmune diseases of the digestive system. Crohn's disease may be localized to any portion of the gastrointestinal tract, including the terminal ileum, and may impact all cell types of the gastrointestinal tract. Ulcerative colitis is localized to the colon and rectum, and affects cells of the mucosa only.

Inflammatory Crohn's disease is characterized by abnormal immune response that causes excess gastrointestinal inflammation. Inflammatory Crohn's disease most often affects the intestinal walls, including portions of the small and/or large intestine, for example, the ileum and the colon. Inflammatory Crohn's disease is also associated with development of thick and swollen tissue, as well as ulcers. Mutations in genes that play a role in immune system function (e.g., NOD2, ATG16L1, IL23R, and IRGM) are associated with Crohn's disease, as are genes associated with proper autophagy.

Fibrostricturing Crohn's disease is a form of Crohn's disease that includes the occurrence of fibrostenotic lesions/fibrotic strictures of the gastrointestinal tract. Fibrotic strictures are more often associated with Crohn's disease of the ileum, as opposed to the colon. Inflammation generally precedes the development strictures. Strictures can be associated with any of the following symptoms: abdominal cramping, abdominal pain, abdominal bloating and distension, loss of appetite, fatigue, nausea, vomiting, and constipation. More than 50% of patients that suffer from Crohn's disease experience fibrostenosis.

Fibrotic strictures are associated with cell surface IL-17A receptor expression and increased collagen production, including collagen subtypes I, III, and V. Fibrotic strictures are also associated with co-occurrence of fistulas. Strictures are histologically characterized by the presence of a mixture of inflammatory and mesenchymal cells with deposition of an excess of extracellular matrix (ECM). Activated mesenchymal cells of the intestine accumulate ECM components, including fibronectin and collagen (for example, collagen types III and IV). Disorganized smooth muscle proliferation and excess ECM deposition are believed to modify the mechanical properties of stenotic bowel tissue, resulting in increased stiffness and narrowing of the intestinal tract associated with fibrotic strictures. The presence of fibrotic strictures results in intestinal tissue abnormalities in both the mucosal, submucosal, and muscular layers of the intestinal wall.

Both environmental and genetic factors are believed to play a role in inflammatory bowel diseases, although the identity of such factors is not well-defined. For example, habitual smoking doubles the risk of developing Crohn's disease. Environmental components may include alterations in flora of the gut which are affected by exposure to ingested foods and medications.

Inflammatory bowel diseases are associated with symptoms including abdominal pain, vomiting, diarrhea, rectal bleeding, severe cramps, muscle spasms, weight loss, malnutrition, fever, anemia, skin lesions, joint pain, eye inflammation, liver disorders, arthritis, pyoderma gangrenosum, primary sclerosing cholangitis, and non-thyroidal illness syndrome, and treating these symptoms using a disclosed antisense compound is also contemplated in an embodiment, for example, treating children suffering from ulcerative colitis who may also suffer from growth defects.

### Intestinal Fibrosis

Intestinal fibrosis commonly results from the reaction of intestinal tissue to inflammation, such as chronic inflammation caused by inflammatory bowel disease (IBD). In IBD, increased levels of SMAD7 block anti-inflammatory gene expression mediated via TGF-β pathway activation and Smad2/3 phosphorylation. Myofibroblasts exposed to increased TGF-β signaling produce increased amounts of collagen. In the context of Crohn's disease, persistent and chronic inflammation promotes fibrotic processes, resulting in the formation of strictures, including small bowel and colonic strictures.

Intestinal fibrosis can be identified by any of a number of imaging techniques, such as contrast-enhanced ultrasonography. See, e.g., Quaia et al. The value of small bowel wall contrast enhancement after sulfur hexafluoride-filled microbubble injection to differentiate inflammatory from fibrotic strictures in patients with Crohn's disease. Ultrasound Med. Biol. 38, 1324-1332 (2012); Nylund et al. Quantitative contrast-enhanced ultrasound comparison between inflammatory and fibrotic lesions in patients with Crohn's disease. Ultrasound Med. Biol. 39, 1197-1206 (2013); Stidham et al. Ultrasound elasticity imaging for detecting intestinal fibrosis and inflammation in rats and humans with Crohn's disease. Gastroenterology 141, 819-826 (2011). MRI techniques such as magnetization transfer MRI can also be used. See, e.g., Maccioni et al. Value of T2-weighted magnetic resonance imaging in the assessment of wall inflammation and fibrosis in Crohn's disease. Abdom. Imaging 37, 944-957 (2012); Adler et al. Magnetization transfer helps detect intestinal fibrosis in an animal model of Crohn disease. Radiology 259, 127-135 (2011); Pazahr et al. Magnetization transfer for the assessment of bowel fibrosis in patients with Crohn's disease: initial experience. Magn. Reson. Mat. Phys. Biol. Med. 26, 291-301 (2013).

### Fibrosis

Methods of preventing or treating fibrosis, such as hepatic fibrosis, pulmonary fibrosis, and/or intestinal fibrosis, form part of this disclosure. Such methods may comprise administering to a patient in need thereof or a patient at risk, a pharmaceutical preparation comprising an IL-34 inhibitor, for example an IL-34 antisense oligonucleotide, disclosed herein. For example, a method of preventing or treating hepatic fibrosis is provided comprising administering to a patient in need thereof an IL-34 inhibitor, for example an IL-34 antisense oligonucleotide, disclosed herein. Alternatively, a method of preventing or treating intestinal fibrosis is provided comprising administering to a patient in need thereof an IL-34 inhibitor, for example an IL-34 antisense oligonucleotide, disclosed herein. Alternatively, a method of preventing or treating pulmonary fibrosis is provided comprising administering to a patient in need thereof an IL-34 inhibitor, for example an IL-34 antisense oligonucleotide, disclosed herein.

Patients treated using an above method may or may not have detectable fibrosis. In some embodiments, the patient has at least about a 5%, 10%, 20%, 30%, 40% or even 50% or more reduction in the amount of fibrosis present in the patient after administering an IL-34 antisense oligonucleotide, after e.g. 1 day, 2 days, 1 week, 1 month or 6 months or more. Administering such an IL-34 inhibitor, for example an IL-34 antisense oligonucleotide, may be on, e.g., at least a daily basis. The compound may be administered orally. The delay of clinical manifestation of fibrosis in a patient as a consequence of administering an IL-34 inhibitor, for example an IL-34 antisense oligonucleotide, disclosed here may be at least e.g., 6 months, 1 year, 18 months or even 2 years or more as compared to a patient who is not administered an IL-34 inhibitor such as one disclosed herein.

A patient in need may have hepatic fibrosis that has developed into cirrhosis. A patient at risk of hepatic fibrosis may include those patients with hepatitis B, hepatitis C or nonalcoholic steatohepatitis (NASH). NASH is included in the spectrum of nonalcoholic fatty liver diseases, including steatosis and cirrhosis. NASH is a component of the metabolic syndrome, which is characterized by obesity, type 2 diabetes mellitus, and dyslipidemia, and can eventually lead to hepatocellular carcinoma.

Methods of treating disorders associated with hepatic fibrosis are also provided, such as the treatment of at least one of: certain storage diseases and inborn errors of metabolism, such as, alpha 1-antitrypsin deficiency, copper storage diseases (e.g., Wilson's disease), fructosemia, galactosemia, glycogen storage diseases (e.g., Types III, IV, VI, IX and X), iron-overload syndromes (e.g., hemochromatosis), lipid abnormalities (e.g. Gaucher's disease), peroxisomal disorders (e.g., Zellweger syndrome), and tyrosinemia; bacterial infections (e.g., brucellosis); parasitic infections (e.g., echinococcosis); NASH; viral infections (e.g., hepatitis B or hepatitis C, including chronic hepatitis B or C); Budd-Chiari syndrome; heart failure; hepatic veno-occlusive disease; and portal vein thrombosis. Methods of treating congenital hepatic fibrosis are also contemplated. The composition may be administered orally.

Abuse of drugs and/or alcohol has been implicated in cases of hepatic fibrosis. Contemplated herein are methods of treating hepatic fibrosis in a patient with a history of drug and/or alcohol abuse. For example, a patient with a history of abusing at least one of the following: alcohol, amiodarone, chlorpromazine, isoniazid, methotrexate, methyldopa, oxyphenisatin and, tolbutamide.

A patient at risk of intestinal fibrosis may include those patients with ulcerative colitis, inflammatory bowel disease, or Crohn's disease. A patient at risk may also include those patients with an early age at diagnosis of Crohn's or colitis, extensive and/or severe of colonic disease, patients with the presence of primary sclerosing cholangitis, and/or patient's having a family history of cancer.

Methods of treating disorders associated with intestinal fibrosis are also provided, such as the treatment of at least one of: ulcerative colitis, an inflammatory bowel disease, or Crohn's disease.

Contemplated herein are methods of preventing or treating renal fibrosis, cardiac fibrosis, endomyocardial fibrosis, idiopathic pulmonary fibrosis, myelofibrosis, retroperitoneal fibrosis, or nephrogenic systemic fibrosis, comprising administering to a patient in need thereof, a pharmaceutical preparation comprising an IL-34 inhibitor, for example an IL-34 antisense oligonucleotide, such as an IL-34 antisense oligonucleotide disclosed herein.

The IL-34 inhibitors of the invention, including, for example, IL-34 AONs, can be used alone or in combination with each other whereby at least two IL-34 inhibitors of the invention are used together in a single composition or as part of a treatment regimen. The IL-34 inhibitors of the invention may also be used in combination with other drugs for treating drug and/or alcohol abuse, renal fibrosis, cardiac fibrosis, endomyocardial fibrosis, idiopathic pulmonary fibrosis, myelofibrosis, retroperitoneal fibrosis, or nephrogenic systemic fibrosis, drug and/or alcohol abuse.

### Treatment and Evaluation

A “patient” or “subject” as described herein, refers to any animal at risk for, suffering from or diagnosed for an inflammatory and/or fibrotic disease, including, but not limited to, mammals, primates, and humans. In certain embodiments, the patient may be a non-human mammal such as, for example, a cat, a dog, or a horse. A patient may be an individual diagnosed with a high risk of developing an inflammatory and/or fibrotic disease, someone who has been diagnosed with an inflammatory and/or fibrotic disease, someone who previously suffered from an inflammatory and/or fibrotic disease, or an individual evaluated for symptoms or indications of an inflammatory and/or fibrotic disease, for example, IL-34 expression signal.

A “patient in need,” as used herein, refers to a patient suffering from any of the symptoms or manifestations of an inflammatory and/or fibrotic disease, a patient who may suffer from any of the symptoms or manifestations of an inflammatory and/or fibrotic disease, or any patient who might benefit from a method of the disclosure for treating an inflammatory and/or fibrotic disease. A patient in need may include a patient who is diagnosed with a risk of developing an inflammatory and/or fibrotic disease, a patient who has suffered from an inflammatory and/or fibrotic disease in the past, or a patient who has previously been treated for an inflammatory and/or fibrotic disease. Of particular relevance are individuals that suffer from an inflammatory and/or fibrotic disease associated with increased levels of IL-34 expression or activity.

The terms “treat”, “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. preventing the disease from increasing in severity or scope; (c) relieving the disease, i.e. causing partial or complete amelioration of the disease; or (d) preventing relapse of the disease, i.e. preventing the disease from returning to an active state following previous successful treatment of symptoms of the disease or treatment of the disease.

An “effective amount” or a “pharmaceutically effective amount,” as used herein, refers to the amount of an agent that is sufficient to at least partially treat a condition when administered to a patient. The therapeutically effective amount will vary depending on the severity of the condition, the route of administration of the component, and the age, weight, etc. of the patient being treated. Accordingly, an effective amount of a disclosed IL-34 antisense oligonucleotide is the amount of the IL-34 antisense oligonucleotide necessary to treat an inflammatory and/or fibrotic disease in a patient such that administration of the agent prevents an inflammatory and/or fibrotic disease from occurring in a subject, prevents an inflammatory and/or fibrotic disease progression (e.g., prevents the onset or increased severity of symptoms of inflammatory bowel disease such as rectal bleeding, anemia, or gastrointestinal inflammation), or relieves or completely ameliorates some or all associated symptoms of an inflammatory and/or fibrotic disease, e.g., causes regression of the disease.

Efficacy of treatment may be evaluated by means of evaluation of gross symptoms associated with an inflammatory and/or fibrotic disease, analysis of tissue histology, biochemical assay, imaging methods such as, for example, magnetic resonance imaging, or other known methods. For instance, efficacy of treatment may be evaluated by analyzing gross symptoms of the disease such as changes in abdominal pain, vomiting, diarrhea, rectal bleeding, cramps, muscle spasms, weight loss, malnutrition, fever, anemia or other aspects of gross pathology associated with an inflammatory disease following administration of a disclosed IL-34 antisense oligonucleotide to a patient suffering from an inflammatory disease.

Efficacy of treatment may also be evaluated at the tissue or cellular level, for example, by means of obtaining a tissue biopsy (e.g., a gastrointestinal tissue biopsy) and evaluating gross tissue or cell morphology or staining properties. Biochemical assays that examine protein or RNA expression may also be used to evaluate efficacy of treatment. For instance, one may evaluate IL-34, IL-6, IL-8, TNF-alpha, or levels of another protein or gene product indicative of an inflammatory and/or fibrotic disease, inflammatory cytokine production, or an IL-34 mediated inflammatory response in dissociated cells or non-dissociated tissue via immunocytochemical, immunohistochemical, Western blotting, or Northern blotting methods, or methods useful for evaluating RNA levels such as quantitative or semi-quantitative polymerase chain reaction. One may also evaluate the presence or level of expression of useful biomarkers found in fecal matter, plasma, or serum to evaluate disease state and efficacy of treatment.

In evaluating efficacy of treatment, suitable controls may be chosen to ensure a valid assessment. For instance, one can compare symptoms evaluated in a patient with an inflammatory and/or fibrotic disease following administration of a disclosed IL-34 inhibitor, for example an IL-34 antisense oligonucleotide, to those symptoms in the same patient prior to treatment or at an earlier point in the course of treatment or in another patient not diagnosed with the inflammatory and/or fibrotic disease. Alternatively, one may compare the results of biochemical or histological analysis of gastrointestinal tissue following administration of a disclosed IL-34 inhibitor, for example an IL-34 antisense oligonucleotide, with those of gastrointestinal tissue from the same patient or from an individual not diagnosed with the inflammatory and/or fibrotic disease or from the same patient prior to administration of the IL-34 inhibitor. Additionally, one may compare blood, serum, cell, or fecal samples following administration of the IL-34 inhibitor, for example an IL-34 antisense oligonucleotide, with comparable samples from an individual not diagnosed with the inflammatory and/or fibrotic disease or from the same patient prior to administration of the IL-34 antisense inhibitor.

Validation of IL-34 inhibition may be determined by direct or indirect assessment of IL-34 expression levels or activity. For instance, biochemical assays that measure IL-34 protein or RNA expression may be used to evaluate overall IL-34 inhibition. For instance, one may measure IL-34 protein levels in gastrointestinal tissue by Western blot to evaluate overall IL-34 levels. One may also measure IL-34 mRNA levels by means of Northern blot or quantitative polymerase chain reaction to determine overall IL-34 inhibition. One may also evaluate IL-34 protein levels or levels of another protein indicative of IL-34 signaling activity in dissociated cells, non-dissociated tissue, blood, serum, or fecal matter via immunocytochemical or immunohistochemical methods.

IL-34 inhibition may also be evaluated indirectly by measuring parameters such as macrophage or monocyte generation or proliferation, or measuring alterations in other parameters correlated with changes in IL-34 activity, including MAP kinase phosphorylation and other indicators of signaling activation of the IL-34 receptor—the macrophage colony stimulating factor receptor (M-CSFR-1, also known as MCSFR-1, M-CSFR1, and CSF1R). For instance, one may measure levels of active MAPK1 or MAPK3 phosphorylation in cells of a patient treated with a disclosed IL-34 inhibitor, for example an IL-34 antisense oligonucleotide, as an indication of IL-34 activity in said cells. One may also evaluate the presence or level of expression of useful biomarkers found in plasma, blood, fecal matter or tissue to evaluate efficacy of IL-34 inhibition.

Methods of treatment disclosed herein include methods of inhibiting inflammatory cytokine production. “Inflammatory cytokine production” refers to the expression of cytokines that initiate and/or promote an inflammatory cytokine response. An “inflammatory cytokine response” refers to an immune response that may be characterized by granulocyte recruitment, lymphocyte recruitment, systemic inflammation (especially of the gastrointestinal tract or a portion or portions thereof), fever, tissue destruction, shock, and/or death. An inflammatory cytokine response may be characterized by binding of individual cytokines to their cognate cell surface receptor (e.g., IL-34 binding to CSF1R) and subsequent cascades of intracellular signaling that alter cell functions and gene expression. Inflammatory cytokines include, but are not limited to IL-1, IL-6, IL-8, IL-34, and TNF-alpha. Expression of inflammatory cytokines may occur in, for example, macrophages, monocytes, propia lamina mononuclear cells, or other cells of the gastrointestinal tract or cells of the immune system. Methods of inhibiting inflammatory cytokine production include methods that reduce expression levels of some or all inflammatory cytokines in a patient suffering from an inflammatory disease. Methods of inhibiting inflammatory cytokine production also include methods that reduce expression levels of some or all inflammatory cytokines in cells of a patient suffering from an inflammatory disease.

Methods of the disclosure for inhibiting inflammatory cytokine production include methods of reducing or inhibiting an IL-34 mediated inflammatory response. An “IL-34 mediated inflammatory response,” as used herein, refers to an inflammatory response initiated, facilitated, or promoted by IL-34 expression or IL-34 signaling activity. An IL-34 mediated inflammatory response may result in expression of inflammatory cytokines including, but not limited to, IL-34, IL-6, IL-8, or TNF-alpha, and activation of inflammatory cytokine signaling. Additionally, an IL-34 mediated inflammatory response may be characterized by granulocyte recruitment, lymphocyte recruitment, systemic inflammation (especially of the gastrointestinal tract or a portion or portions thereof), fever, tissue destruction, shock, and/or death. An IL-34 mediated inflammatory response may also be characterized by activation of IL-34 signaling, for instance, binding of IL-34 to CSF1R and phosphorylation of downstream MAP kinases. Reducing or inhibiting an IL-34 mediated inflammatory response refers to alleviating any or all of the cellular and systemic changes associated with an IL-34 mediated inflammatory response. For example, a reduction in inflammatory cytokine production, immune cell recruitment, or tissue inflammation would indicate reducing or inhibiting of an IL-34 mediated inflammatory response.

The disclosure also provides methods of inhibiting IL-34 in cells of a patient suffering from an inflammatory and/or fibrotic disease. IL-34 may be inhibited in any cell in which IL-34 expression or activity occurs, including cells of the gastrointestinal tract, immune system, and blood. Cells of the gastrointestinal tract (including cells of the intestinal wall, the stomach, duodenum, jejunum, large intestine, small intestine, ileum, colon, rectum and anal canal), include columnar epithelial cells, intestinal stromal cells, mucosal epithelial cells, zymogenic cells, neck mucus cells, parietal cells, gastrin cells, Goblet cells, Paneth cells, oligomucus cells, and villus absorptive cells. Cells of the immune system include leukocytes, phagocytes (e.g., macrophages, neutrophils, and dendritic cells), monocytes, mast cells, eosinophils, basophils, natural killer cells, innate cells, lymphocytes, B cells, and T cells. Blood cells include red blood cells (erythrocytes) and white blood cells (leukocytes, monocytes, and platelets).

### Pharmaceutical Compositions and Routes of Administration

The present disclosure also provides methods for treating an inflammatory and/or fibrotic disease via administration of a pharmaceutical composition comprising a disclosed IL-34 inhibitor, for example an IL-34 antisense oligonucleotide. In another aspect, the disclosure provides a pharmaceutical composition for use in treating an inflammatory and/or fibrotic disease. The pharmaceutical composition may be comprised of a disclosed IL-34 inhibitor, for example an IL-34 antisense oligonucleotide, that targets IL-34 and a pharmaceutically acceptable excipient. As used herein the term “pharmaceutical composition” means, for example, a mixture containing a specified amount of a therapeutic compound, e.g., a therapeutically effective amount, of a therapeutic compound in a pharmaceutically acceptable excipient to be administered to a mammal, e.g., a human, in order to treat an inflammatory and/or fibrotic disease. In some embodiments, contemplated herein are pharmaceutical compositions comprising a disclosed IL-34 inhibitor, for example an IL-34 antisense oligonucleotide, and a pharmaceutically acceptable excipient. In another aspect, the disclosure provides use of a disclosed IL-34 inhibitor, for example, an IL-34 antisense oligonucleotide, in the manufacture of a medicament for treating an inflammatory and/or fibrotic disease. “Medicament,” as used herein, has essentially the same meaning as the term “pharmaceutical composition.”

As used herein, “pharmaceutically acceptable excipient” refers to a substance that aids the administration of an active agent to and/or absorption by a subject and can be included in the compositions of the present invention without causing a significant adverse toxicological effect on the patient. Non-limiting examples of pharmaceutically acceptable excipients include water, NaCl, normal saline solutions, such as a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with IL-34 inhibitors of the invention. For examples of excipients, see Martin, Remington's Pharmaceutical Sciences, 15th Ed., Mack Publ. Co., Easton, Pa. (1975).

In one embodiment, a disclosed IL-34 inhibitor, for example an IL-34 antisense oligonucleotide, and any pharmaceutical composition thereof may be administered by one or several routes, including topically, parenterally, orally, pulmonarily, intratracheally, intranasally, transdermally, or intraduodenally. The term parenteral as used herein includes subcutaneous injections, intrapancreatic administration, intravenous, intramuscular, intraperitoneal, intrasternal injection or infusion techniques. For example, a disclosed IL-34 inhibitor, for example an IL-34 antisense oligonucleotide, may be administered subcutaneously to a subject. In another example, a disclosed IL-34 inhibitor, for example an IL-34 antisense oligonucleotide, may be administered orally to a subject. In another example, a disclosed IL-34 inhibitor, for example an IL-34 antisense oligonucleotide, may be administered directly to the gastrointestinal system, or specific regions of the gastrointestinal system (e.g., the ileum, colon, or rectum) via parenteral administration.

Pharmaceutical compositions containing an IL-34 inhibitor, for example an IL-34 antisense oligonucleotide, such as those disclosed herein, can be presented in a dosage unit form and can be prepared by any suitable method. A pharmaceutical composition should be formulated to be compatible with its intended route of administration. Useful formulations can be prepared by methods well known in the pharmaceutical art. For example, see Remington's Pharmaceutical Sciences, 18th ed. (Mack Publishing Company, 1990).

Pharmaceutical formulations, for example, are sterile. Sterilization can be accomplished, for example, by filtration through sterile filtration membranes. Where the composition is lyophilized, filter sterilization can be conducted prior to or following lyophilization and reconstitution.

Parenteral Administration

The pharmaceutical compositions of the disclosure can be formulated for parenteral administration, e.g., formulated for injection via the intravenous, intramuscular, subcutaneous, intralesional, or intraperitoneal routes. The preparation of an aqueous composition, such as an aqueous pharmaceutical composition containing a disclosed IL-34 inhibitor, for example an IL-34 antisense oligonucleotide, will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. In addition, sterile, fixed oils may be employed as a solvent or suspending medium. For this purpose any bland fixed oil can be employed including synthetic mono- or diglycerides. In addition, fatty acids such as oleic acid can be used in the preparation of injectables. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In one embodiment, a disclosed IL-34 inhibitor may be suspended in a carrier or excipient fluid comprising 1% (w/v) sodium carboxymethylcellulose and 0.1% (v/v) TWEEN™ 80. Under ordinary conditions of storage and use, these preparations contain a preservative to prevent the growth of microorganisms.

Injectable preparations, for example, sterile injectable aqueous or oleaginous suspensions may be formulated according to the known art using suitable dispersing or wetting agents and suspending agents. Generally, dispersions are prepared by incorporating the various sterilized active ingredients into a sterile vehicle which contains the basic dispersion medium and the required other ingredients from those enumerated above. Sterile injectable solutions of the disclosure may be prepared by incorporating a disclosed IL-34 inhibitor, for example an IL-34 antisense oligonucleotide, in the required amount of the appropriate solvent with various of the other ingredients enumerated above, as required, followed by filtered sterilization. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The injectable formulations can be sterilized, for example, by filtration through a bacteria-retaining filter.

The preparation of more, or highly concentrated solutions for intramuscular injection is also contemplated. In this regard, the use of DMSO as solvent is preferred as this will result in extremely rapid penetration, delivering high concentrations of the disclosed IL-34 antisense oligonucleotide to a small area.

Suitable preservatives for use in such a solution include benzalkonium chloride, benzethonium chloride, chlorobutanol, thimerosal and the like. Suitable buffers include boric acid, sodium and potassium bicarbonate, sodium and potassium borates, sodium and potassium 10 carbonate, sodium acetate, sodium biphosphate and the like, in amounts sufficient to maintain the pH at between about pH 6 and pH 8, and for example, between about pH 7 and pH 7.5. Suitable tonicity agents are dextran 40, dextran 70, dextrose, glycerin, potassium chloride, propylene glycol, sodium chloride, and the like, such that the sodium chloride equivalent of the solution is in the range 0.9 plus or minus 0.2%. Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxymethylpropylcellulose, lanolin, methylcellulose, petrolatum, polyethylene glycol, polyvinyl alcohol, polyvinylpyrrolidone, carboxymethylcellulose and the like.

Oral Administration

In some embodiments, contemplated herein are compositions suitable for oral delivery of a disclosed IL-34 inhibitor, for example an IL-34 antisense oligonucleotide, e.g., tablets that include an enteric coating, e.g., a gastro-resistant coating, such that the compositions may deliver the IL-34 inhibitor to, e.g., the gastrointestinal tract of a patient. For example, such administration may result in a topical effect, substantially topically applying the IL-34 inhibitor directly to an affected portion of the gastrointestinal tract of a patient. Such administration, may, in some embodiments, substantially avoid unwanted systemic absorption of the IL-34 inhibitor.

For example, a tablet for oral administration is provided that comprises granules (e.g., is at least partially formed from granules) that include a disclosed IL-34 inhibitor, for example an IL-34 antisense oligonucleotide, e.g., an antisense oligonucleotide that includes the nucleotide sequence of any one of SEQ ID NOs:1-23, and pharmaceutically acceptable excipients. Such a tablet may be coated with an enteric coating. Contemplated tablets may include pharmaceutically acceptable excipients such as fillers, binders, disintegrants, and/or lubricants, as well as coloring agents, release agents, coating agents, sweetening, flavoring such as wintergreen, orange, xylitol, sorbitol, fructose, and maltodextrin, and perfuming agents, preservatives and/or antioxidants.

In some embodiments, contemplated pharmaceutical formulations include an intra-granular phase that includes a disclosed IL-34 inhibitor, for example an IL-34 antisense oligonucleotide, e.g., an antisense oligonucleotide that includes the nucleotide sequence of any one of SEQ ID NOs:1-23, and a pharmaceutically acceptable salt, and a pharmaceutically acceptable filler. For example, a disclosed IL-34 inhibitor, for example an IL-34 antisense oligonucleotide, and a filler may be blended together, optionally, with other excipients, and formed into granules. In some embodiments, the intragranular phase may be formed using wet granulation, e.g. a liquid (e.g., water) is added to the blended IL-34 inhibitor and filler, and then the combination is dried, milled and/or sieved to produce granules. One of skill in the art would understand that other processes may be used to achieve an intragranular phase.

In some embodiments, contemplated formulations include an extra-granular phase, which may include one or more pharmaceutically acceptable excipients, and which may be blended with the intragranular phase to form a disclosed formulation.

A disclosed formulation may include an intragranular phase that includes a filler. Exemplary fillers include, but are not limited to, cellulose, gelatin, calcium phosphate, lactose, sucrose, glucose, mannitol, sorbitol, microcrystalline cellulose, pectin, polyacrylates, dextrose, cellulose acetate, hydroxypropylmethyl cellulose, partially pre-gelatinized starch, calcium carbonate, and others including combinations thereof.

In some embodiments, a disclosed formulation may include a intragranular phase and/or a extragranular phase that includes a binder, which may generally function to hold the ingredients of the pharmaceutical formulation together. Exemplary binders of the disclosure may include, but are not limited to, the following: starches, sugars, cellulose or modified cellulose such as hydroxypropyl cellulose, lactose, pre-gelatinized maize starch, polyvinyl pyrrolidone, hydroxypropyl cellulose, hydroxypropylmethyl cellulose, low substituted hydroxypropyl cellulose, sodium carboxymethyl cellulose, methyl cellulose, ethyl cellulose, sugar alcohols and others including combinations thereof.

Contemplated formulations, e.g., that include an intragranular phase and/or an extragranular phase, may include a disintegrant such as but are not limited to, starch, cellulose, crosslinked polyvinyl pyrrolidone, sodium starch glycolate, sodium carboxymethyl cellulose, alginates, corn starch, crosmellose sodium, crosslinked carboxymethyl cellulose, low substituted hydroxypropyl cellulose, acacia, and others including combinations thereof. For example, an intragranular phase and/or an extragranular phase may include a disintegrant.

In some embodiments, a contemplated formulation includes an intra-granular phase comprising a disclosed IL-34 inhibitor, for example an IL-34 antisense oligonucleotide, and excipients chosen from: mannitol, microcrystalline cellulose, hydroxypropylmethyl cellulose, and sodium starch glycolate or combinations thereof, and an extra-granular phase comprising one or more of: microcrystalline cellulose, sodium starch glycolate, and magnesium stearate or mixtures thereof.

In some embodiments, a contemplated formulation may include a lubricant, e.g. an extra-granular phase may contain a lubricant. Lubricants include but are not limited to talc, silica, fats, stearin, magnesium stearate, calcium phosphate, silicone dioxide, calcium silicate, calcium phosphate, colloidal silicon dioxide, metallic stearates, hydrogenated vegetable oil, corn starch, sodium benzoate, polyethylene glycols, sodium acetate, calcium stearate, sodium lauryl sulfate, sodium chloride, magnesium lauryl sulfate, talc, and stearic acid.

In some embodiments, the pharmaceutical formulation comprises an enteric coating. Generally, enteric coatings create a barrier for the oral medication that controls the location at which the drug is absorbed along the digestive track. Enteric coatings may include a polymer that disintegrates at different rates according to pH. Enteric coatings may include for example, cellulose acetate phthalate, methyl acrylate-methacrylic acid copolymers, cellulose acetate succinate, hydroxylpropylmethyl cellulose phthalate, methyl methacrylate-methacrylic acid copolymers, ethylacrylate-methacrylic acid copolymers, methacrylic acid copolymer type C, polyvinyl acetate-phthalate, and cellulose acetate phthalate.

Exemplary enteric coatings include Opadry® AMB, Acryl-EZE, Eudragit® grades. In some embodiments, an enteric coating may comprise about 5% to about 10%, about 5% to about 20%, 8 to about 15%, about 8% to about 20%, about 10% to about 20%, or about 12 to about 20%, or about 18% of a contemplated tablet by weight. For example, enteric coatings may include an ethylacrylate-methacrylic acid copolymer.

For example, in a contemplated embodiment, a tablet is provided that comprises or consists essentially of about 0.5% to about 70%, e.g. about 0.5% to about 10%, or about 1% to about 20%, by weight of a disclosed IL-34 inhibitor, for example an IL-34 antisense oligonucleotide, or a pharmaceutically acceptable salt thereof. Such a tablet may include for example, about 0.5% to about 60% by weight of mannitol, e.g. about 30% to about 50% by weight mannitol, e.g. about 40% by weight mannitol; and/or about 20% to about 40% by weight of microcrystalline cellulose, or about 10% to about 30% by weight of microcrystalline cellulose. For example, a disclosed tablet may comprise an intragranular phase that includes about 30% to about 60%, e.g. about 45% to about 65% by weight, or alternatively, about 5 to about 10% by weight of a disclosed IL-34 inhibitor, for example an IL-34 antisense oligonucleotide, about 30% to about 50%, or alternatively, about 5% to about 15% by weight mannitol, about 5% to about 15% microcrystalline cellulose, about 0% to about 4%, or about 1% to about 7% hydroxypropylmethylcellulose, and about 0% to about 4%, e.g. about 2% to about 4% sodium starch glycolate by weight.

In another contemplated embodiment, a pharmaceutical tablet formulation for oral administration of a disclosed IL-34 inhibitor, for example an IL-34 antisense oligonucleotide, comprises an intra-granular phase, wherein the intra-granular phase includes a disclosed IL-34 inhibitor or a pharmaceutically acceptable salt thereof (such as a sodium salt), and a pharmaceutically acceptable filler, and which may also include an extra-granular phase, that may include a pharmaceutically acceptable excipient such as a disintegrant. The extra-granular phase may include components chosen from microcrystalline cellulose, magnesium stearate, and mixtures thereof. The pharmaceutical composition may also include an enteric coating of about 12% to 20% by weight of the tablet. For example, a pharmaceutically acceptable tablet for oral use may comprise about 0.5% to 10% by weight of a disclosed IL-34 inhibitor, for example an IL-34 antisense oligonucleotide, or a pharmaceutically acceptable salt thereof, about 30% to 50% by weight mannitol, about 10% to 30% by weight microcrystalline cellulose, and an enteric coating comprising an ethylacrylate-methacrylic acid copolymer.

In another example, a pharmaceutically acceptable tablet for oral use may comprise an intra-granular phase, comprising about 5 to about 10% by weight of a disclosed IL-34 inhibitor, for example an IL-34 antisense oligonucleotide, or a pharmaceutically acceptable salt thereof, about 40% by weight mannitol, about 8% by weight microcrystalline cellulose, about 5% by weight hydroxypropylmethyl cellulose, and about 2% by weight sodium starch glycolate; an extra-granular phase comprising about 17% by weight microcrystalline cellulose, about 2% by weight sodium starch glycolate, about 0.4% by weight magnesium stearate; and an enteric coating over the tablet comprising an ethylacrylate-methacrylic acid copolymer.

In some embodiments the pharmaceutical composition may contain an enteric coating comprising about 13% or about 15%, 16%, 17% or 18% by weight, e.g., AcyrlEZE® (see, e.g., PCT Publication No. WO2010/054826, which is hereby incorporated by reference in its entirety).

The rate at which point the coating dissolves and the active ingredient is released is its dissolution rate. In an embodiment, a contemplated tablet may have a dissolution profile, e.g. when tested in a USP/EP Type 2 apparatus (paddle) at 100 rpm and 37° C. in a phosphate buffer with a pH of 7.2, of about 50% to about 100% of the IL-34 inhibitor releasing after about 120 minutes to about 240 minutes, for example after 180 minutes. In another embodiment, a contemplated tablet may have a dissolution profile, e.g. when tested in a USP/EP Type 2 apparatus (paddle) at 100 rpm and 37° C. in diluted HCl with a pH of 1.0, where substantially none of the IL-34 inhibitor is released after 120 minutes. A contemplated tablet, in another embodiment, may have a dissolution profile, e.g. when tested in USP/EP Type 2 apparatus (paddle) at 100 rpm and 37° C. in a phosphate buffer with a pH of 6.6, of about 10% to about 30%, or not more than about 50%, of the IL-34 inhibitor releasing after 30 minutes.

Contemplated formulations, e.g. tablets, in some embodiments, when orally administered to the patient may result in minimal plasma concentration of the IL-34 inhibitor, for example an IL-34 antisense oligonucleotide, in the patient. In another embodiment, disclosed formulations, when orally administered to a patient, topically deliver to the colon or rectum of a patient, e.g. to an affected or diseased site of a patient.

In some embodiments, methods provided herein may further include administering at least one other agent that is directed to treatment of diseases and disorders disclosed herein. In one embodiment, contemplated other agents may be co-administered (e.g., sequentially or simultaneously).

Agents contemplated include immunosuppressive agents including glucocorticoids, cytostatics, antibodies, agents acting on immunophilins, interferons, opioids, TNF binding proteins, mycophenolate, and small biological agents. For example, contemplated immunosuppressive agents include, but are not limited to: tacrolimus, cyclosporine, pimecrolimus, sirolimus, everolimus, mycophenolic acid, fingolimod, dexamethasone, fludarabine, cyclophosphamide, methotrexate, azathioprine, leflunomide, teriflunomide, anakinra, anti-thymocyte globulin, anti-lymphocyte globulin, muromonab-CD3, afutuzumab, rituximab, teplizumab, efalizumab, daclizumab, basiliximab, adalimumab, infliximab, certolizumab pegol, natalizumab, and etanercept. Other contemplated agents include antibiotics, anti-diarrheals, laxatives, pain relievers, iron supplements, and calcium or vitamin D or B-12 supplements.

### Dosage and Frequency of Administration

Exemplary formulations include dosage forms that include or consist essentially of about 35 mg to about 500 mg of a disclosed IL-34 inhibitor, for example an IL-34 antisense oligonucleotide. For example, formulations that include about 35 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, or 250 mg of a disclosed IL-34 inhibitor, for example an IL-34 antisense oligonucleotide, are contemplated herein. In one embodiment, a formulation may include about 40 mg, 80 mg, or 160 mg of a disclosed IL-34 inhibitor, for example an IL-34 antisense oligonucleotide. In some embodiments, a formulation may include at least 100 μg of a disclosed IL-34 inhibitor, for example an IL-34 antisense oligonucleotide. For example, formulations may include about 0.1 mg, 0.2 mg, 0.3 mg, 0.4 mg, 0.5 mg, 1 mg, 5 mg, 10 mg, 15 mg, 20 mg, or 25 mg of a disclosed IL-34 inhibitor, for example an IL-34 antisense oligonucleotide. The amount administered will depend on variables such as the type and extent of disease or indication to be treated, the overall health and size of the patient, the in vivo potency of the IL-34 inhibitor, the pharmaceutical formulation, and the route of administration. The initial dosage can be increased beyond the upper level in order to rapidly achieve the desired blood-level or tissue level. Alternatively, the initial dosage can be smaller than the optimum, and the dosage may be progressively increased during the course of treatment. Human dosage can be optimized, e.g., in a conventional Phase I dose escalation study designed to run from 40 mg to 160 mg. Dosing frequency can vary, depending on factors such as route of administration, dosage amount and the disease being treated. Exemplary dosing frequencies are once per day, once per week and once every two weeks. In some embodiments, dosing is once per day for 7 days.

### Diagnostic Methods

The disclosure also provides a method of diagnosing a patient with an inflammatory and/or fibrotic disease that relies upon detecting levels of IL-34 expression signal in one or more biological samples of a patient. As used herein, the term “IL-34 expression signal” can refer to any indication of IL-34 gene expression, or gene or gene product activity. IL-34 gene products include RNA (e.g., mRNA), peptides, and proteins. Indices of IL-34 gene expression that can be assessed include, but are not limited to, IL-34 gene or chromatin state, IL-34 gene interaction with cellular components that regulate gene expression, IL-34 gene product expression levels (e.g., IL-34 RNA expression levels, IL-34 protein expression levels), or interaction of IL-34 RNA or protein with transcriptional, translational, or post-translational processing machinery. Indices of IL-34 gene product activity include, but are not limited to, assessment of IL-34 signaling activity (e.g., assessment of CSF1R activation or MAPK1/MAPK3 phosphorylation) and assessment of IL-34 receptor binding (e.g., CSF1R binding).

Detection of IL-34 expression signal may be accomplished through in vivo, in vitro, or ex vivo methods. In a preferred embodiment, methods of the disclosure may be carried out in vitro. Methods of detecting may involve detection in blood, serum, fecal matter, tissue, or cells of a patient. Detection may be achieved by measuring IL-34 expression signal in whole tissue, tissue explants, cell cultures, dissociated cells, cell extract, or body fluids, including blood or serum. Contemplated methods of detection include assays that measure levels of IL-34 gene product expression such as Western blotting, FACS, ELISA, other quantitative binding assays, cell or tissue growth assays, Northern blots, quantitative or semi-quantitative polymerase chain reaction, medical imaging methods (e.g., MRI), or immunostaining methods (e.g., immunohistochemistry or immunocytochemistry).

### EXAMPLES

The disclosure is further illustrated by the following examples. The examples are provided for illustrative purposes only, and are not to be construed as limiting the scope or content of the disclosure in any way.

### Example 1: The HT-29 CRC Cell Lines Expresses IL-34 at High Levels

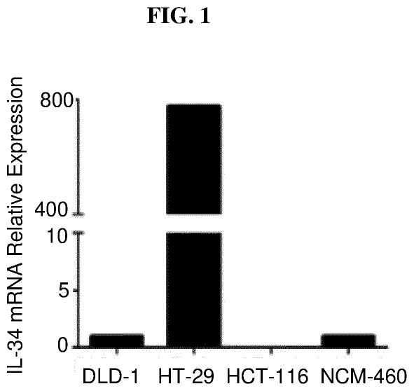

To evaluate IL-34 expression in human CRC cell lines, IL-34 mRNA expression was analyzed by RT-PCR in the DLD-1, HT-29, and HCT-116 human CRC cell lines, and in the NCM-460 normal intestinal epithelial cell line. IL-34 mRNA expression levels were normalized using (β-actin mRNA levels as a reference. As shown in FIG. 1, IL-34 mRNA expression was abundant in HT-29 cells.

### Example 2: Design and Evaluation of IL-34 Antisense Oligonucleotides

IL-34 antisense oligonucleotides of SEQ ID NOs:1-4 complementary to mouse and human IL-34 mRNA sequences were designed and produced with phosphorothioate backbones. Knockdown of IL-34 expression was evaluated in HT-29 human and RAW 264.7 mouse cell lines. HT-29 cells (ATCC Manassas, Va., USA) were cultured in McCoy's 5A (Lonza, Verviers, Belgium) supplemented with 10% FBS, 1% P/S (all from Lonza) and maintained at 37° C. with 5% CO₂ in a humidified incubator. Murine RAW264.7 macrophages (ATCC Manassas, Va., USA) were cultured in Dulbecco's modified Eagle's medium (1 g/L of glucose) with 10% FBS, 1% P/S and 1% non-essential amino acid (NEA) (all from Lonza) and maintained at 37° C. with 5% CO₂ in a humidified incubator. Cells were either left untreated or transfected with a specific IL-34 AON or scrambled AON using Opti-MEM medium and Lipofectamine 3000 reagent (Life Technologies, Milan, Italy) according to the manufacturer's instructions. Total RNA and protein were extracted.

Knockdown was evaluated by real-time polymerase chain reaction (RT-PCR) in HT-29 cells. RT-PCR was performed as follows: a constant amount of RNA (0.5 μg/sample) was retro-transcribed into complementary DNA (cDNA). 1 μl of cDNA/sample was then amplified using the following protocol: denaturation for 1 minute at 95° C.; annealing for 30 seconds at 60° C., followed by 30 seconds of extension at 72° C. The following primer sequences were used: IL-34 forward, 5′-ACAGGAGCCGACTTCAGTAC-3′ (SEQ ID NO:29); IL-34 reverse, 5′-ACCAAGACCCACAGATACCG-3′ (SEQ ID NO:30); β-actin forward, 5′-AAGATGACCCAGATCATGTTTGAGACC-3′ (SEQ ID NO:31); and β-actin reverse, 5′-AGCCAGTCCAGACGCAGGAT-3′ (SEQ ID NO:32). mRNA expression was calculated relative to β-Actin mRNA expression.

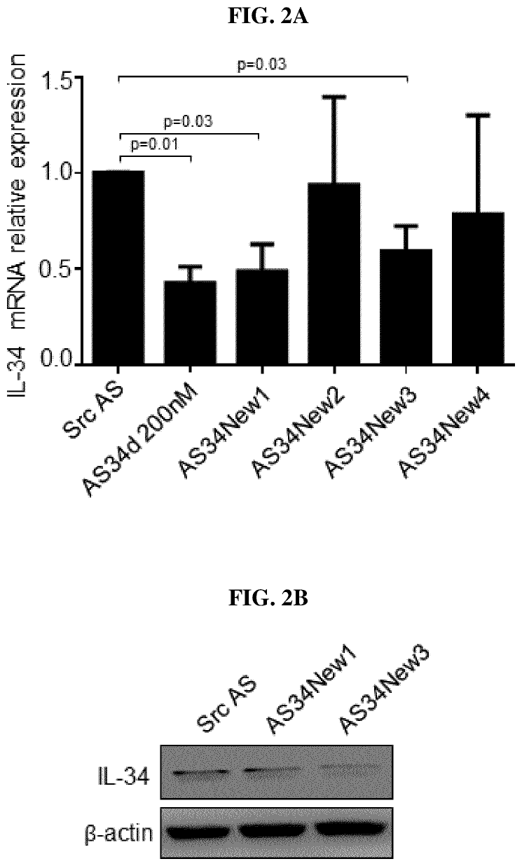

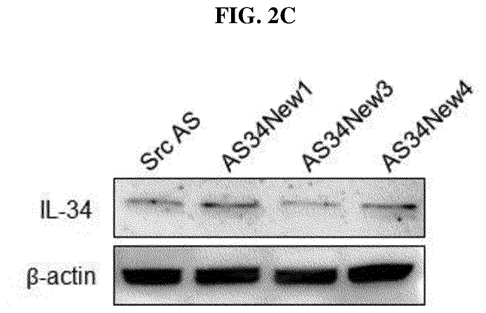

HT-29 cells were transfected with human IL-34 AON's of SEQ ID NO:1, 2, 3, or 4, or a scrambled negative control oligonucleotide (SRC AS; 5′-AACACGTCTATACGC-3′ (SEQ ID NO:33)) for 24 hours (FIG. 2A, AS34New1-4, equivalent to SEQ ID NOs:1-4, respectively). IL-34 transcripts were evaluated by RT-PCR and values were normalized to β-actin. Transfection of HT-29 cells with IL-34 AON's of SEQ ID NOs:1 and 3 significantly decreased IL-34 mRNA expression as evaluated by RT-PCR (FIG. 2A; Src AS v. AS34New1, p=0.03; Src AS v. AS34New3, p=0.03; data are expressed as mean±standard error of the mean (SEM)).

Knockdown was also evaluated by Western blotting in HT-29 cells and RAW 264.7 cells. Western blotting was performed as follows: following transfection, cells were lysed on ice in buffer containing 10 mM HEPES (pH 7.9), 10 mM KCl, 0.1 mM EDTA, 0.2 mM EGTA, and 0.5% Nonidet P40 supplemented with 1 mM dithiothreitol, 10 mg/ml aprotinin, 10 mg/ml leupeptin, 1 mM phenylmethylsulfonyl fluoride, 1 mM Na3VO4 and 1 mM NaF. Lysates were clarified by centrifugation at 4° C., 12.000×g for 30 minutes, and separated on 10% sodium dodecyl sulphate-polyacrylamide gel electrophoresis. IL-34 was detected using mouse anti human IL-34 antibody (1:1,000 final dilution; Abcam Cambridge, UK), in combination with horseradish peroxidase (HRP)-conjugated secondary IgG monoclonal antibody (1:20,000 final dilution; Dako, Milan, Italy). HRP reaction was detected with a sensitive enhanced chemiluminescence kit (Pierce, Rockford, Ill.). After the analysis, blots were stripped and probed with mouse anti-human (β-actin antibody (1:5,000 final dilution; Sigma-Aldrich), followed by HRP-conjugated secondary IgG monoclonal antibody (1:20,000 final dilution; Dako).

FIG. 2B shows that transfection of HT-29 cells with IL-34 scrambled control or AS34New1 or 3 IL-34 AONs resulted in decreased IL-34 protein expression. FIG. 2C shows that transfection of RAW 264.7 cells with IL-34 scrambled control or AS34New1 or 3 IL-34 AONs resulted in decreased IL-34 protein expression.

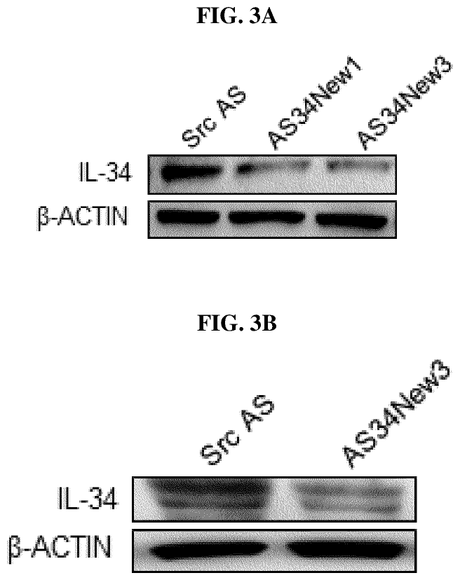

IL-34 knockdown by IL-34 AONs AS34New1 and 3 was further evaluated in fibrostricturing Crohn's disease and cancer associated fibroblasts. Intestinal fibroblasts were isolated from intestinal colon cancer or fibrostricturing CD (FS CD) explants. Fibroblasts were used freshly or between passages 3 and 8. Fibroblasts were cultured in DMEM high glucose with UltraGlutamine supplemented with 10% FBS, 1% P/S (Lonza) and maintained at 37° C. with 5% CO₂ in a humidified incubator. Fibroblasts were detached using Trypsin/EDTA solution. Transfection of FS CD explants with AS34New1 or 3 resulted in visibly increased knockdown of IL-34 compared to FS CD explants transfected with Src AS (FIG. 3A). Transfection of cancer-associated fibroblasts with AS34New3 resulted in visibly increased knockdown of IL-34 compared to fibroblasts transfected with Src AS (FIG. 3B).

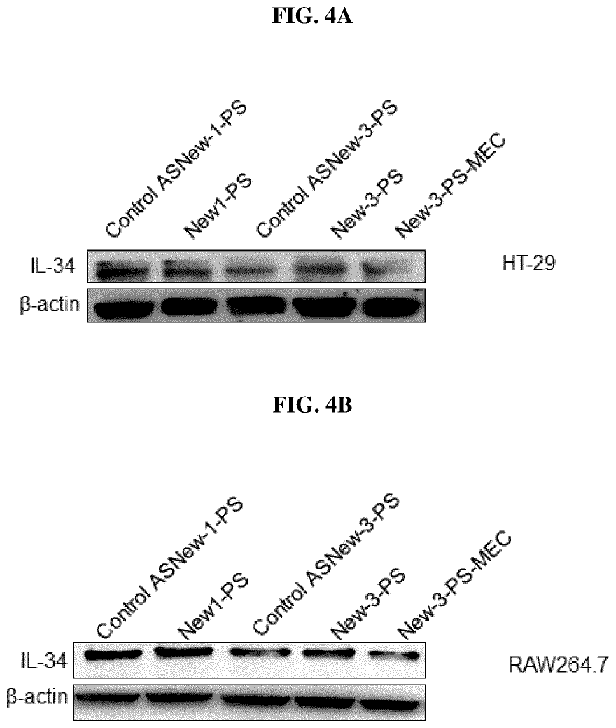

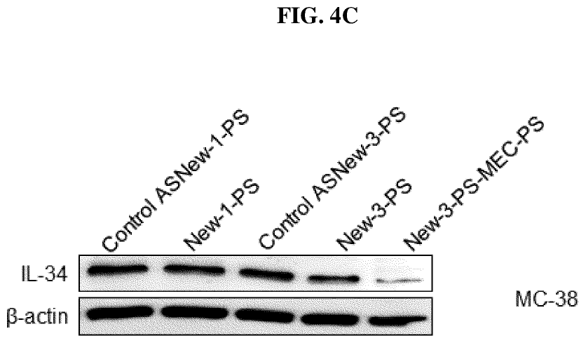

Knockdown efficacy of an IL-34 AON that includes a 5-methylcytosine modification (IL-34 AON of SEQ ID NO:7) was tested in HT-29, RAW 264.7, and MC-38 cell lines. MC38 is a murine cell line. MC-38 cells (ATCC Manassas, Va., USA) were cultured in McCoy's 5A, supplemented with 10% of FBS and 1% of P/S (Lonza) and maintained at 37° C. with 5% CO₂ in a humidified incubator. All cell lines were transfected with IL-34 AONs for 24 hours, after which, IL-34 and β-actin levels were analyzed by Western blotting. Cells were transfected with IL-34 AON's with a phosphorothioate backbone of SEQ ID NO:1, 3, or 7 (New1-PS, New-3-PS, New-3-PS-MEC, respectively; FIGS. 4A, 4B, 4C) or scrambled controls (Control ASNew-1-PS, Control ASNew-3-PS). Transfection with an IL-34 AON of SEQ ID NO:7 that includes a 5-methylcytosine resulted in increased IL-34 knockdown relative to transfection with all other AONs tested (FIGS. 4A, 4B, and 4C). These results demonstrate that IL-34 AONs, including IL-34 AONs of SEQ ID NO:7 were able to knock down expression of IL-34 mRNA and protein in mouse and human cell lines.

### Example 3: M-CSFR-1 is Over-Expressed in Crohn's Disease Fibrostrictures

Expression of M-CSFR-1 was evaluated in ileal inflammatory CD (I CD) and FS-CD specimens from CD patients by RT-PCR. Surgical specimens were taken from 10 patients with I CD undergoing surgery for chronic active disease with poor responsiveness to medical treatment and from 27 patients with FS CD undergoing surgery. Ileal controls (CTR) were mucosal specimens taken from macroscopically and microscopically unaffected areas of 27 patients undergoing surgery for colon cancer. Each patient who took part in the study gave written informed consent and the study protocol was approved by the local Ethics Committees.

RT-PCR was performed as follows: RNA (0.5 μg/sample) was retro-transcribed into cDNA. 1 μl of cDNA/sample was amplified using the following conditions: denaturation for 1 minute at 95° C.; annealing for 30 seconds at 58° C. for M-CSFR-1, or 60° C. for (β-Actin, followed by 30 seconds of extension at 72° C. The following primer sequences were used: M-CSFR-1 forward, 5′-CTGCTCAACTTTCTGCGAAG-3′ (SEQ ID NO:34); M-CSFR-1 reverse, 5′-CTCATCTCCACATAGGTGTC-3′(SEQ ID NO:35); (β-actin forward, 5′-AAGATGACCCAGATCATGTTTGAGACC-3′ (SEQ ID NO:31); and β-actin reverse, 5′-AGCCAGTCCAGACGCAGGAT-3′ (SEQ ID NO:32). mRNA expression was calculated relative to β-Actin mRNA expression.

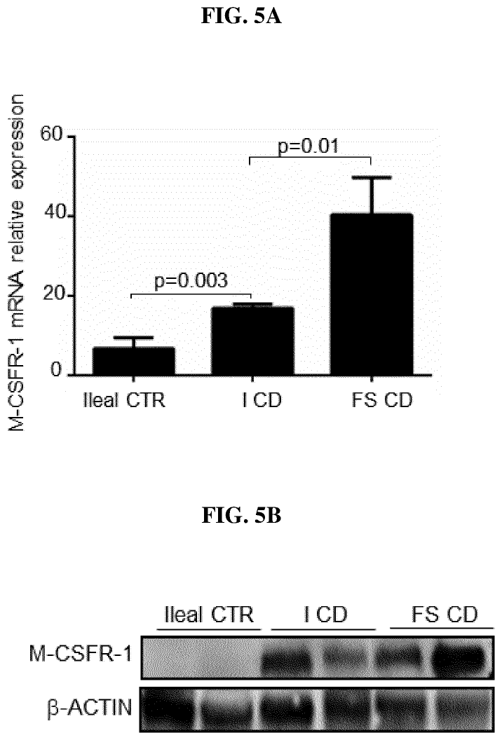

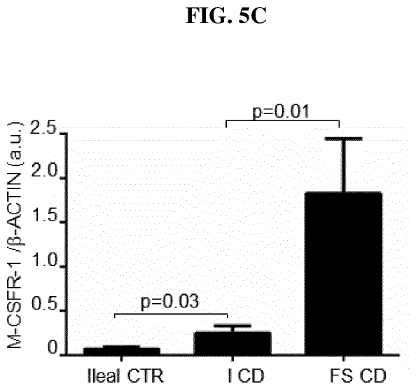

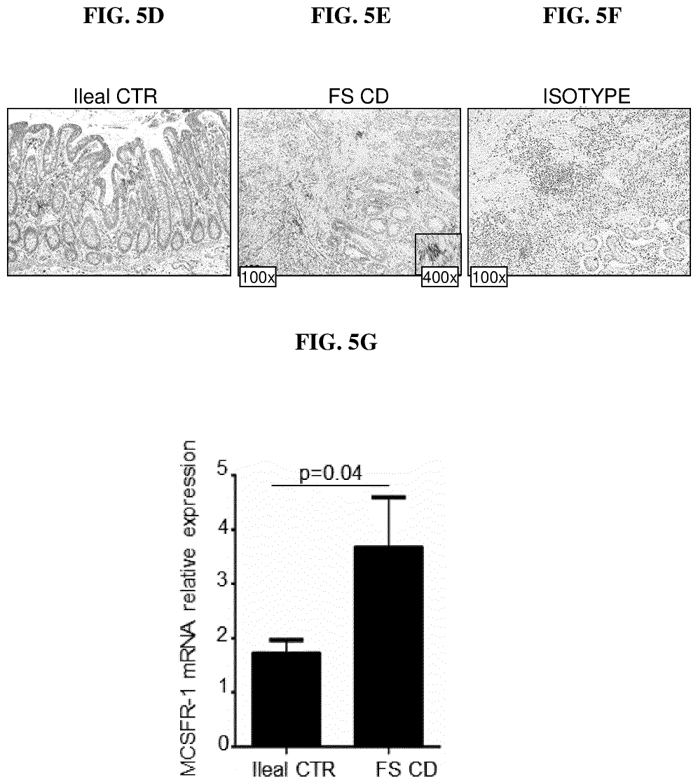

RT-PCR to evaluate M-CSFR-1 expression was performed as described above in control, I CD, and FS CD samples. M-CSFR-1 mRNA transcripts were increased in I CD patients as compared to CTR (FIG. 5A). Additionally, M-CSFR-1 mRNA transcripts were increased in FS CD samples compared to I CD and ileal CTR samples (FIG. 5A; Ileal CTR v. I CD, p=0.003; I CD v. FS CD, p=0.01; mean±SEM).

Western blotting of I CD and FS CD samples was performed to evaluate M-CSFR-1 expression. Cells were lysed on ice in buffer containing 10 mM HEPES (pH 7.9), 10 mM KCl, 0.1 mM EDTA, 0.2 mM EGTA, and 0.5% Nonidet P40 supplemented with 1 mM dithiothreitol, 10 mg/ml aprotinin, 10 mg/ml leupeptin, 1 mM phenylmethylsulfonyl fluoride, 1 mM Na3VO4, and 1 mM NaF. Lysates were clarified by centrifugation at 4° C. for 30 minutes and separated on a 10% sodium dodecyl sulphate-polyacrylamide by gel electrophoresis. After protein transfer to a membrane, membranes were incubated with rabbit anti-human M-CSFR-1 (1:500, Novus Biological Southpark Way, USA), followed by HRP-conjugated secondary IgG monoclonal antibody (final dilution of 1:20,000, Dako, Milan, Italy). The HRP reaction was detected with a sensitive enhanced chemiluminescence kit (Pierce, Rockford, Ill.). After the analysis, blots were stripped and incubated with mouse anti-human (β-Actin (final dilution of 1:5,000, Sigma-Aldrich). Computer-assisted scanning densitometry (Image-Lab 5.2.1, Bio-Rad Laboratories, Milan, Italy) was used to analyze the intensity of the immunoreactive bands.

Protein extraction and Western blot detection of M-CSFR-1 in I CD, FS CD, and CTR samples confirmed that M-CSFR-1 expression in I CD is greater than that of CTR, and expression in FS CD is increased relative to both CTR and I CD samples (FIGS. 5B and 5C; Ileal CTR v. I CD, p=0.03; I CD v. FS CD, p=0.01; mean±SEM).

Expression of M-CSFR-1 was further evaluated by immunohistochemistry. Immunohistochemistry was performed on formalin-fixed, paraffin-embedded ileal sections of ileal CTR, I CD patients, and FS CD patients. Sections were deparaffinized and dehydrated with xylene and ethanol, and the antigen retrieval was performed in Tris EDTA citrate buffer (pH 7.8) in a thermostatic bath at 98° C. (Dako) for 30 minutes. Immunohistochemical staining was performed using rabbit monoclonal antibody directed against human M-CSFR-1 (final concentration 1:200, Novus Biological) incubated at room temperature (RT) for 1 hour followed by detection using a biotin-free HRP-polymer detection technology with 3,3′diaminobenzidine (DAB) as a chromogen (MACH 4 Universal HRP-Polymer Kit, Biocare Medical). The sections were counterstained with haematoxylin, dehydrated and mounted. Isotype control IgG-stained sections were prepared under identical immunohistochemical conditions as described above, replacing the primary antibody with a purified mouse normal IgG control antibody (R&D Systems).

Immunohistochemical analysis of ileal control and FS CD samples showed enhanced expression of M-CSFR-1 in FS CD (FIG. 5E) compared to control sections (FIG. 5D). M-CSFR-1 expression was not observed in isotype controls (FIG. 5F). Immunohistochemical analysis also demonstrated that stromal cells were positive for M-CSFR-1 (FIG. 5E). These results were further confirmed by RT-PCR, which showed increased M-CSFR-1 mRNA expression in fibroblasts isolated from ileal samples of FS CD patients as compared to control fibroblasts (FIG. 5G; Ileal CTR v. FS CD, p=0.04; mean±SEM)). These results demonstrate that expression of M-CSFR-1—the receptor for IL-34—was increased in tissue of CD patients relative to ileal tissue from non-CD patients. Furthermore, these results demonstrated that M-CSFR-1 expression is higher in FS CD tissue as opposed to tissue from I CD patients.

### Example 4: IL-34 Induced Collagen Production in Crohn's Disease Fibroblasts

Regulation of collagen production in gut fibroblasts by IL-34 expression was evaluated by stimulating control intestinal fibroblasts with IL-34. Fibroblasts were used between passages 3 and 8. Fibroblasts were maintained in 75 cm² plastic flasks and incubated at 37° C. in a humidified atmosphere with 5% CO₂ in D-MEM containing high glucose with Ultra Glutamine and supplemented with 10% fetal bovine serum (FBS), 1% of penicillin (100 U/ml), streptomycin (100 μg/ml), and 1% of non-essential amino acids (Lonza; Verviers, Belgium). 5×10⁴ normal fibroblasts were plated into each well of a 12-well plate, left to adhere for 24 hours, starved for 6 hours, and finally either left unstimulated or stimulated with recombinant human IL-34 (50 ng/ml, R&D Systems, Minneapolis, Minn.). After 6-48 hours, cells and cell-free supernatants were harvested. Cells were used for protein and gene expression analysis and cell-free supernatants were analyzed for the content of collagen. The following primers were used for RT-PCR of COL1A1 and COL3A1: COL1A1 forward, 5′-GGACACAGAGGTTTCAGTGG-3′ (SEQ ID NO:36); COL1A1 reverse, 3′-GGTGACTTTGGAGACACAGG-5 (SEQ ID NO:37); COL3A1 forward 5′-GGAGAATGTTGTGCAGTTTGC-3′(SEQ ID NO:38); and COL3A1 reverse, 3′-CGTTTGACGTGTTGTAAGAGG-5′ (SEQ ID NO:39). The following antibodies were used for Western blotting: rabbit anti-human COL1A1 (final dilution 1:1,000; Novus Biological, Italy) and rabbit anti-human COL3A1 (final dilution 1:1,000; Novus Biological)

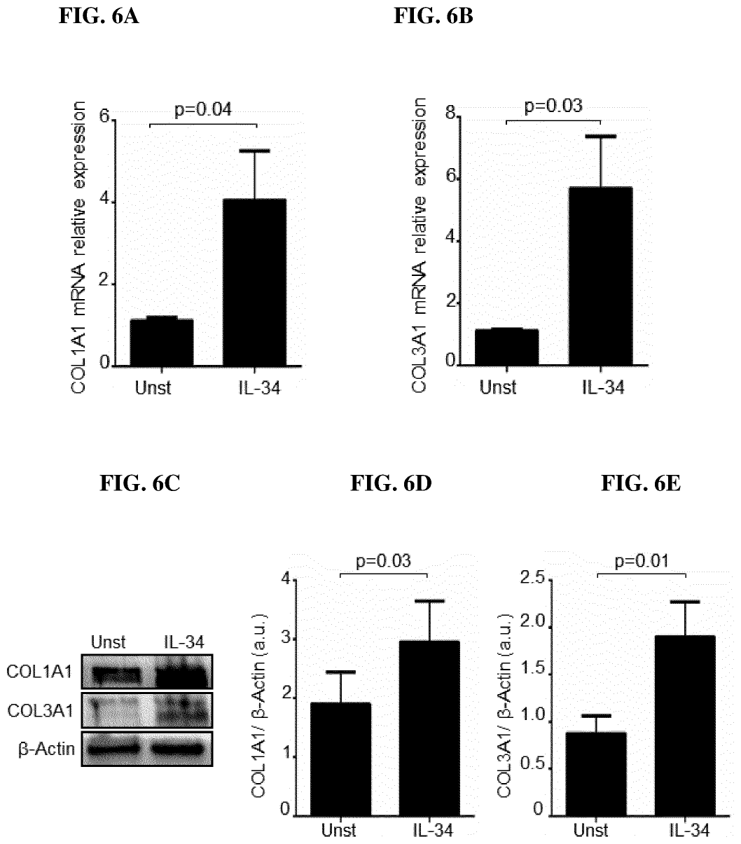

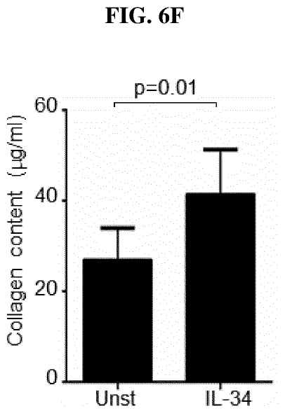

RT-PCR analysis demonstrated that stimulation with recombinant human IL-34 significantly enhanced COL1A1 and COL3A1 mRNA transcript production in fibroblasts compared to unstimulated fibroblasts (Unst; FIG. 6A, Unst v. IL-34, p=0.04; FIG. 6B, Unst v. IL-34, p=0.03; mean±SEM). FIG. 6C is a western blot showing that stimulation with recombinant human IL-34 also significantly enhanced COL1A1 and COL3A1 protein expression in fibroblasts compared to unstimulated fibroblasts. FIGS. 6D and 6E, respectively, are bar graphs showing quantitation of COL1A1 and COL3A1 signal of the western blot shown in FIG. 6C normalized to a (β-actin control (COL1A1, Unst v. IL-34, p=0.03; COL3A1, Unst v. IL-34, p=0.01; mean±SEM).

Total collagen was also measured in fibroblast supernatants using a Sircol Collagen Assay Kit in accordance with the manufacturer's instructions (Biocolor Ltd, Belfast, UK). Quantification of soluble forms of collagen in fibroblast supernatants demonstrated that stimulation with IL-34 resulted in increased collagen synthesis relative to unstimulated controls (FIG. 6F; Unst v. IL-34, p=0.01; mean±SEM). These results demonstrate that IL-34 stimulated collagen production in fibroblast cells.

### Example 5: IL-34 Stimulates Collagen Production Via a p38 MAP Kinase Dependent Pathway

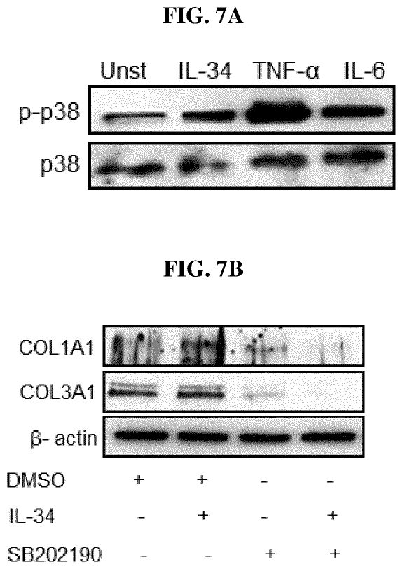

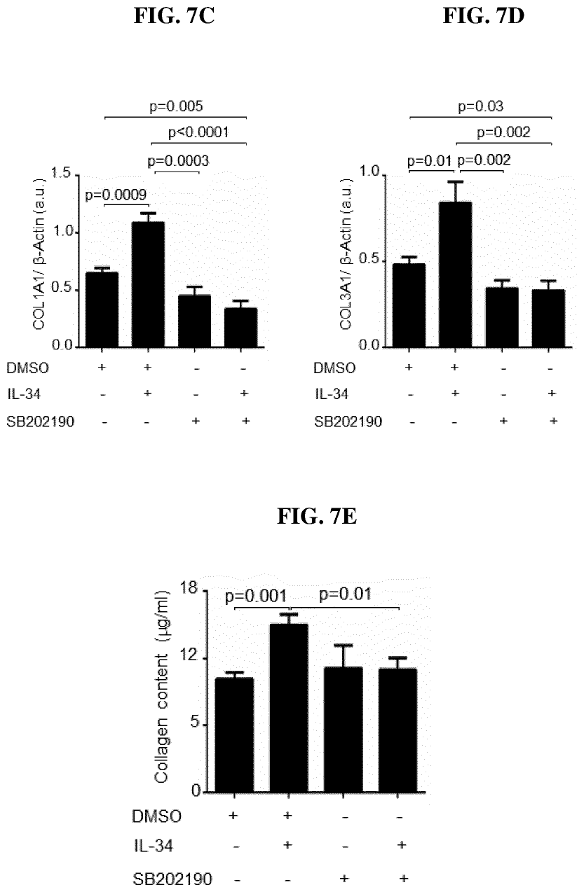

The dependence of IL-34 on collagen synthesis upon p38 MAP kinase was investigated. IL-34 activates p38 MAP kinase in epithelial cells, a signaling pathway that controls intestinal fibroblast function. Control serum-starved intestinal fibroblasts were either left unstimulated or stimulated with recombinant human IL-34 (50 ng/mL), TNF-α (25 ng/ml, R&D Systems), or IL-6 (50 ng/ml, R&D Systems) for 30 minutes, and then lysed. TNF-α and IL-6 served as positive controls. Total extracts were analyzed for the content of both phosphorylated (p-p38) and total p38 mitogen-activated protein (MAP) kinase by Western blotting. Western blotting was performed using the following antibodies: rabbit anti-human p-p38 (1:1,000 EMD Millipore Corporation) and mouse anti-human total p38 (final dilution 1:500, Santa Cruz Biotechnology, Inc., Texas, USA). IL-34 stimulation enhanced phosphorylation of p38 MAP kinase in fibroblasts compared to unstimulated cells (FIG. 7A).

To evaluate the effect of MAP Kinase p38 on IL-34-induced collagen production, normal fibroblasts were preincubated with SB202190, an inhibitor of p38 (10 mol/l; EMD Millipore Corporation, MA, USA) or dimethyl sulfoxide (DMSO; vehicle) for 1 hour and then stimulated or left unstimulated with IL-34 (50 ng/ml) for 24-48 hours. Protein expression in cells was analyzed by Western blotting, and cell-free supernatants were analyzed for collagen content. FIG. 7B is a western blot showing that pre-incubation of cells with SB202190 abrogated IL-34-induced COL1A1 and COL3A1 production. FIGS. 7C and 7D, respectively, are bar graphs showing quantitation of COL1A1 and COL3A1 signal of the western blot shown in FIG. 7B normalized to a (β-actin control (COL1A1 (DMSO v. DMSO+IL-34, p=0.0009; DMSO+IL-34 v. SB202190, p=0.0003; DMSO+IL-34 v. SB202190+IL-34, p=0.0001; DMSO v. SB202190+IL-34, p=0.05); COL3A1 (DMSO v. DMSO+IL-34, p=0.01; DMSO+IL-34 v. SB202190, p=0.002; DMSO+IL-34 v. SB202190+IL-34, p=0.002; DMSO v. SB202190+IL-34, p=0.03); mean±SEM) as evaluated by Western blotting and total collagen production in fibroblasts as evaluated by Sircol assay (FIG. 7E; DMSO v. DMSO+IL-34, p=0.001; DMSO+IL-34 v. SB202190+IL-34, p=0.01; mean±SEM). These results demonstrate that IL-34 induced collagen production is dependent upon the p38 MAP Kinase pathway.

### Example 6: IL-34 Production in Crohn's Disease Fibrostrictures

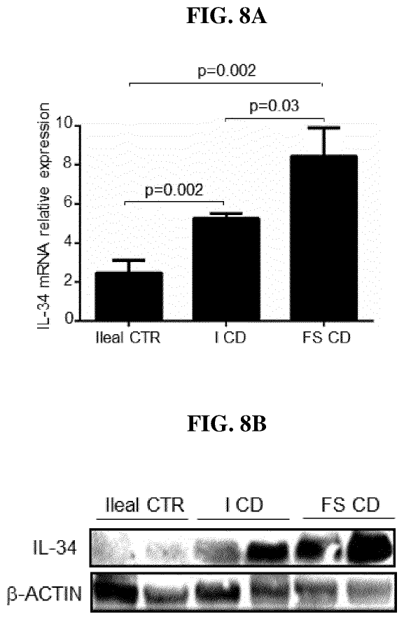

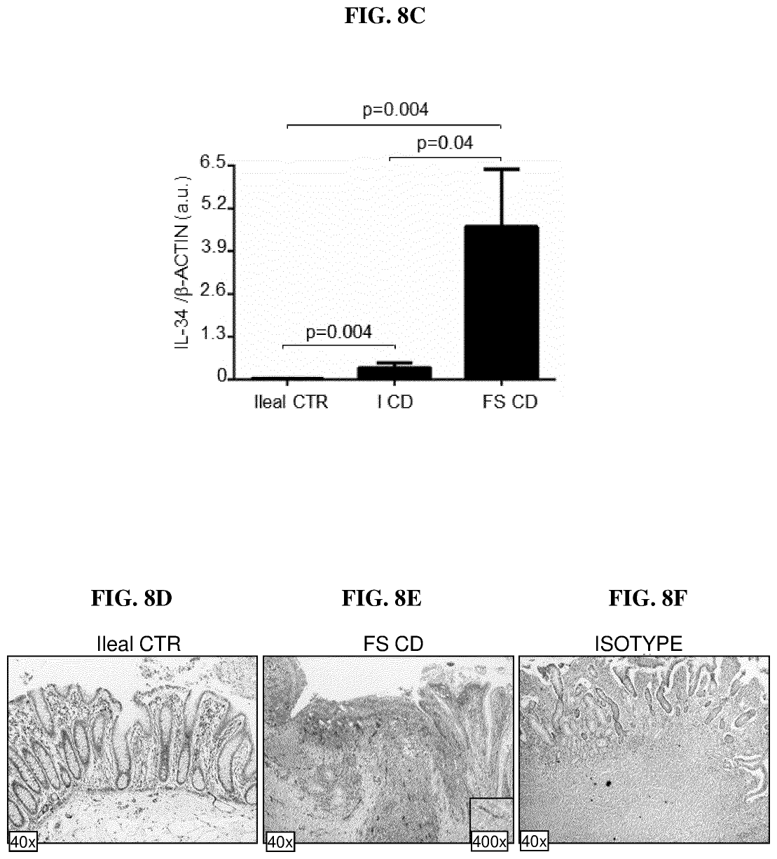

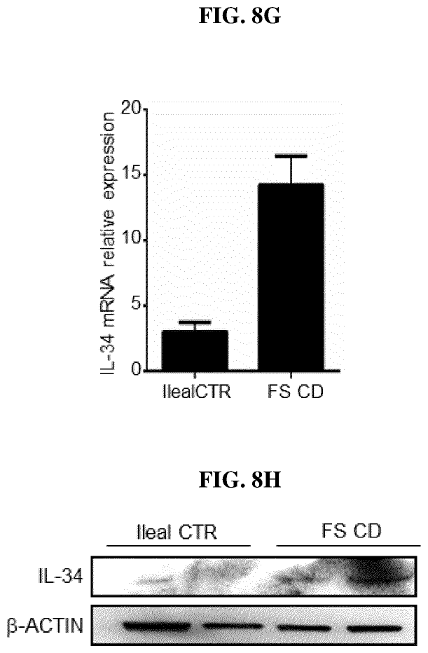

IL-34 expression was evaluated in fibrostrictures to determine what cells of fibrostrictures produce IL-34. RNA transcripts for IL-34 were evaluated in intestinal specimens taken from I CD, FS CD and controls. IL-34 RNA transcripts were significantly increased in I CD patients as compared to controls (FIG. 8A). IL-34 RNA expression was increased in FS CD samples as compared to control and I CD samples (FIG. 8A; Ileal CTR v. I CD, p=0.002; I CD v. FS CD, p=0.03; Ileal CTR v. FS CD, p=0.002; mean±SEM). FIG. 8B is a western blot analysis of total proteins extracted from paired mucosal samples showing that IL-34 expression is significantly increased in I CD patients as compared to controls and in FS CD patients as compared to both control and I CD cells. FIG. 8C is a bar graph showing quantitation of IL-34 signal of the western blot shown in FIG. 8B normalized to a (β-actin control (Ileal CTR v. I CD, p=0.004; I CD v. FS CD, p=0.04; Ileal CTR v. FS CD, p=0.004; mean±SEM). Immunohistochemical analysis also demonstrated that IL-34 is highly expressed in FS CD cells (FIG. 8E) compared to ileal control (FIG. 8D) and isotype control (FIG. 8F). Immunohistochemistry also demonstrated that in FS CD fibrostrictures, stromal cells expressed IL-34 (FIG. 8E). Enhanced IL-34 RNA (FIG. 8G; mean±SEM) and protein expression (FIG. 8H) were observed in fibroblasts isolated from ileal samples of FS CD patients as compared to control fibroblasts.

### Example 7: IL-34 Knockdown in Crohn's Disease Fibroblasts Results in Reduced Collagen Production

The effect of IL-34 inhibition on collagen synthesis in CD fibroblasts isolated from FS specimens was evaluated using an IL-34 antisense oligonucleotide. In brief, 5×10⁴ FS CD fibroblasts were plated into each well of a 12-well plate, left to adhere for 24 hours, and then either left untreated or transfected with an IL-34 antisense oligonucleotide of SEQ ID NO:3 (IL-34 AS) or a complementary sense oligonucleotide (NCAS; both used at 200 nM; Integrated DNA Technologies, Inc. Leuven, Belgium) for 24-48 hours using Opti-MEM medium and Lipofectamine 3000 reagent according to the manufacturer's instructions (Life Technologies, Milan, Italy). Transfection efficiency was determined by Western blotting. Cell-free supernatants were analyzed for collagen content after 48 hours.

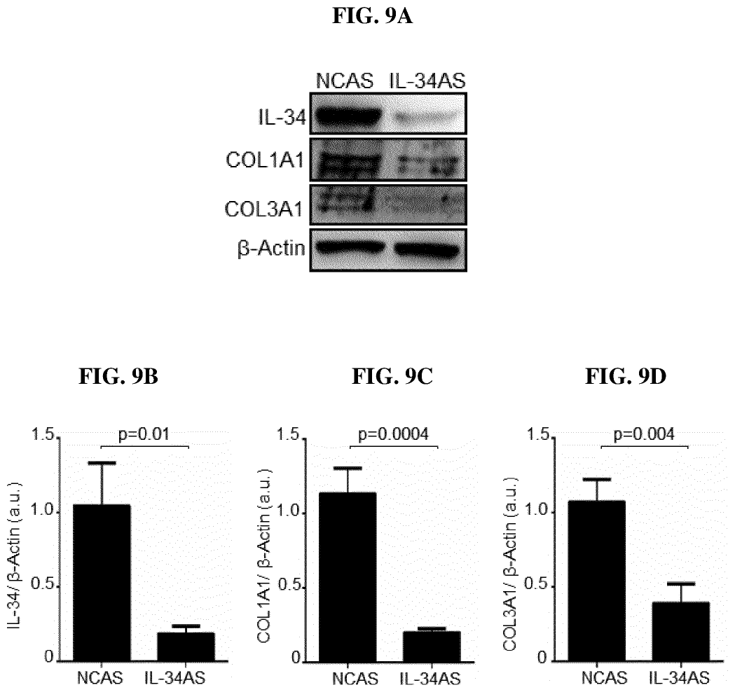

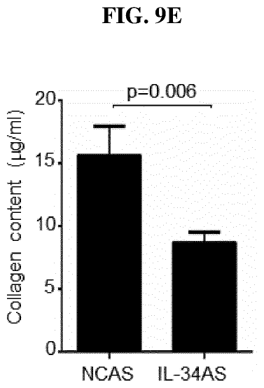

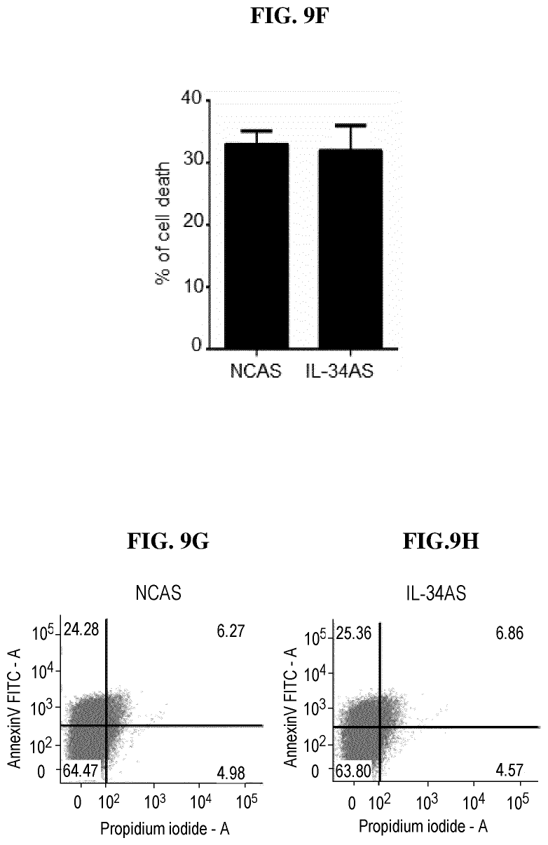

Transfection of FS CD fibroblasts with the IL-34 antisense oligonucleotide significantly reduced both COL1A1 and COL3A1 protein synthesis compared to transfection with a control antisense sequence, as evaluated by Western blotting (FIG. 9A).

FIGS. 9B, 9C, and 9D, respectively, are bar graphs showing quantitation of IL-34, COL1A1, and COL3A1 signal of the western blot shown in FIG. 9A normalized to a (β-actin control; IL-34, NCAS v. IL-34AS, p=0.01; COL1A1, NCAS v. IL-34AS, p=0.0004; COL3A1, NCAS v. IL-34AS, p=0.004; mean±SEM). Transfection of FS CD fibroblasts with the IL-34 antisense oligonucleotide also significantly reduced secretion of collagen in fibroblast culture supernatants (FIG. 9E; NCAS v. IL-34AS, p=0.006, mean±SEM).

The effect of IL-34 knockdown on cell death was also evaluated. To score cell death, FS fibroblasts were washed in PBS, stained with FITC-annexin V (AV, 1:100 final dilution; Immunotools, Friesoyte, Germany) according to the manufacturer's instructions and incubated with 5 mg/ml PI (Life Technologies) for 20 min at 4° C. Fluorescence was measured by flow cytometry using FL-1 and FL-2 channels of a FACSVerse (BD Biosciences) flow cytometer. AV−/PI-cells were considered to be viable cells, AV+/PI-cells were considered to be apoptotic, and secondary necrotic cells were characterized by AV+/PI+ staining. FIGS. 9G and 9H are flow cytometry dot plots of FS CD fibroblasts transfected with IL-34 antisense oligonucleotide or NCAS. FIG. 9F is a bar graph corresponding to the flow cytometry data of FIGS. 9G and 9H (mean±SEM), showing that FS CD fibroblasts transfected with the IL-34 antisense oligonucleotide did not result in increased cell death. These results demonstrate that IL-34 knockdown in FS CD fibroblasts by an antisense oligonucleotide results in significantly decreased collagen production without having a significant effect on cell death.

### INCORPORATION BY REFERENCE

The entire disclosure of each of the patent documents and scientific articles cited herein is incorporated by reference for all purposes.

### EQUIVALENTS

The disclosure can be embodied in other specific forms with departing from the essential characteristics thereof. The foregoing embodiments therefore are to be considered illustrative rather than limiting on the disclosure described herein. The scope of the disclosure is indicated by the appended claims rather than by the foregoing description, and all changes that come within the meaning and range of equivalency of the claims are intended to be embraced therein.

What is claimed is:

1. An IL-34 antisense oligonucleotide comprising a nucleotide sequence selected from the group consisting of:
5′-CTCACCAAGACCCACAG-3′ (SEQ ID NO:1), wherein at least one nucleoside is a chemically modified nucleoside and/or at least one linkage is a modified internucleoside linkage;
5′-GGCTTTGGGCCGCACCAGCT-3′ (SEQ ID NO:2), wherein at least one nucleoside is a chemically modified nucleoside and/or at least one linkage is a modified internucleoside linkage;
5′-CTTTGGGCCGCACCAGCTTC-3′ (SEQ ID NO:3), wherein at least one nucleoside is a chemically modified nucleoside and/or at least one linkage is a modified internucleoside linkage;
5′-TGGGCCGCACCAGCTTCAGG-3′ (SEQ ID NO:4), wherein at least one nucleoside is a chemically modified nucleoside and/or at least one linkage is a modified internucleoside linkage;
5′-TCCATGACCCGGAAGCAGTT-3′ (SEQ ID NO:5), wherein at least one nucleoside is a chemically modified nucleoside and/or at least one linkage is a modified internucleoside linkage; and
5′-TGTTTCATGTACTGAAG-3′ (SEQ ID NO:6), wherein at least one nucleoside is a chemically modified nucleoside and/or at least one linkage is a modified internucleoside linkage,
or
a pharmaceutically acceptable salt thereof.

2. The antisense oligonucleotide of claim 1, wherein the nucleotide sequence is SEQ ID NO:3, and wherein at least one cytidine is chemically modified.

3. The antisense oligonucleotide of claim 2, wherein the nucleotide sequence is 5′-CTTTGGGCXGCACCAGCTTC-3′ (SEQ ID NO:7), and wherein X is 5-methylcytidine.

4. The antisense oligonucleotide of claim 1, wherein the nucleotide sequence is SEQ ID NO:5, and wherein at least one cytidine is chemically modified; optionally wherein the cytidine at position 10 of SEQ ID NO:5 is chemically modified and the nucleotide sequence is 5′-TCCATGACCXGGAAGCAGTT-3′ (SEQ ID NO:8), and wherein X is 5-methylcytidine.

5. The antisense oligonucleotide of any one of claims 1-4, wherein the antisense oligonucleotide comprises one or more locked nucleic acids (LNA).

6. The antisense oligonucleotide of any one of claims 1-5, wherein each of the nucleotides of the antisense oligonucleotide comprises a locked nucleic acid (LNA).

7. An IL-34 antisense oligonucleotide of comprising a nucleotide sequence selected from the group consisting of:
5′-cttTGGGCXGCACCAGCttc-3′ (SEQ ID NO:9), wherein c is LNA cytidine, t is LNA thymidine, and X is 5-methylcytidine;
5′-ctttGGGCXGCACCAGcttc-3′ (SEQ ID NO:10), wherein c is LNA cytidine, t is LNA thymidine, and X is 5-methylcytidine;
5′-cttTGGGCcgCACCAGCttc-3′ (SEQ ID NO:11), wherein c is LNA cytidine, t is LNA thymidine, and g is LNA guanosine;
5′-cttTGGGCcGCACCAGCttc-3′ (SEQ ID NO:12), wherein c is LNA cytidine and t is LNA thymidine;
5′-ggcXGCACCAGCttc-3′ (SEQ ID NO:13), wherein c is LNA cytidine, t is LNA thymidine, g is LNA guanosine, and X is 5-methylcytidine;
5′-cttTGGGCXGCACcag-3′ (SEQ ID NO:14), wherein c is LNA cytidine, t is LNA thymidine, g is LNA guanosine, a is LNA adenosine, and X is 5-methylcytidine; and
5′-tgaCCXGGAAGCAgtt-3′ (SEQ ID NO:15), wherein a is LNA adenosine, t is LNA thymidine, g is LNA guanosine, and X is 5-methylcytidine, or
a pharmaceutically acceptable salt thereof.

8. An IL-34 antisense oligonucleotide of comprising a nucleotide sequence selected from the group consisting of:
5′-CxTxTxTxGxGGCXGCACCAGxCxTxTxCx-3′ (SEQ ID NO:16), wherein Cx is 2′-O-(2-methoxyethyl)cytidine, Tx is 2′-O-(2-methoxyethyl)thymidine, Gx is 2′-O-(2-methoxyethyl)guanosine, and X is 5-methylcytidine;
5′-TxCxCxAxTxGACCXGGAAGCxAxGxTxTx-3′ (SEQ ID NO:17), wherein Cx is 2′-O-(2-methoxyethyl)cytidine, Tx is 2′-O-(2-methoxyethyl)thymidine, Gx is 2′-O-(2-methoxyethyl)guanosine, Ax is 2′-O-(2-methoxyethyl)adenosine, and X is 5-methylcytidine;
5′-CxTxTxTxGxGxGCXGCACCAxGxCxTxTxCx-3′ (SEQ ID NO:18), wherein Cx is 2′-O-(2-methoxyethyl)cytidine, Tx is 2′-O-(2-methoxyethyl)thymidine, Gx is 2′-O-(2-methoxyethyl)guanosine, Ax is 2′-O-(2-methoxyethyl)adenosine, and X is 5-methylcytidine;
5′-TxCxCxAxTxGxACCXGGAAGxCxAxGxTxTx-3′ (SEQ ID NO:19), wherein Cx is 2′-O-(2-methoxyethyl)cytidine, Tx is 2′-O-(2-methoxyethyl)thymidine, Gx is 2′-O-(2-methoxyethyl)guanosine, Ax is 2′-O-(2-methoxyethyl)adenosine, and X is 5-methylcytidine;
5′-CxTxTxTxGxGxGxCxXGxCxAxCxCxAxGxCxTxTxCx-3′ (SEQ ID NO:20), wherein Cx is 2′-O-(2-methoxyethyl)cytidine, Tx is 2′-O-(2-methoxyethyl)thymidine, Gx is 2′-O-(2-methoxyethyl)guanosine, Ax is 2′-O-(2-methoxyethyl)adenosine, and X is 5-methylcytidine; and
5′-TxCxCxAxTxGxAxCxCxXGxGxAxAxGxCxAxGxTxTx-3′ (SEQ ID NO:21), wherein Cx is 2′-O-(2-methoxyethyl)cytidine, Tx is 2′-O-(2-methoxyethyl)thymidine, Gx is 2′-O-(2-methoxyethyl)guanosine, Ax is 2′-O-(2-methoxyethyl)adenosine, and X is 5-methylcytidine, or
a pharmaceutically acceptable salt thereof.

9. The antisense oligonucleotide of any one of claims 1-8, wherein the antisense oligonucleotide comprises one or more ribonucleotides.

10. The antisense oligonucleotide of any one of claims 1-8, wherein the antisense oligonucleotide comprises one or more deoxyribonucleotides.

11. The antisense oligonucleotide of any one of claims 1-8, wherein the antisense oligonucleotide comprises a mixture of ribonucleotides and deoxyribonucleotides.

12. The antisense oligonucleotide of any one of claim 1-11, wherein at least one internucleoside linkage of the sequence is selected from the group consisting of a phosphorothioate linkage, a phosphorodithioate linkage, a phosphotriester linkage, an alkylphosphonate linkage, an aminoalkylphosphotriester linkage, an alkylene phosphonate linkage, a phosphinate linkage, a phosphoramidate linkage, and an aminoalkylphosphoramidate linkage, a thiophosphoramidate linkage, thionoalkylphosphonate linkage, a thionoalkylphosphotriester linkage, a thiophosphate linkage, a selenophosphate linkage, and a boranophosphate linkage.

13. The antisense oligonucleotide of any one of claims 1-12, wherein at least one internucleoside linkage of the sequence is a phosphorothioate linkage.

14. The antisense oligonucleotide any one of claims 1-13, wherein all internucleoside linkages of the sequence are phosphorothioate linkages.

15. The antisense oligonucleotide of any one of claims 1-14, wherein one or more cytidines are replaced with 5-methylcytidine.

16. The antisense oligonucleotide of any one of claims 1-15 wherein the antisense oligonucleotide is 20 nucleotides in length or 20-25, 20-30, 20-35, 25-30, 25-35, or 30-35 nucleotides in length.

17. The antisense oligonucleotide of claim 16, wherein the antisense oligonucleotide is from 20-25 nucleotides in length.

18. The antisense oligonucleotide of any one of claims 1-15, wherein the antisense oligonucleotide is no more than 20, 25, 30, or 35 nucleotides in length.

19. A compound comprising an antisense oligonucleotide of any one of claims 1-18 or a pharmaceutically acceptable salt thereof.

20. A compound comprising an antisense oligonucleotide of any one of claims 1-3 and 5-18, wherein the antisense oligonucleotide comprises the nucleotide sequence of SEQ ID NO:3, and wherein at least one cytidine is chemically modified, or a pharmaceutically acceptable salt thereof.

21. The compound of claim 20, wherein the antisense oligonucleotide comprises the nucleotide sequence 5′-CTTTGGGCXGCACCAGCTTC-3′ (SEQ ID NO:7), and wherein X is 5-methylcytidine.

22. A method of treating an inflammatory disease, the method comprising administering to a patient in need thereof an effective amount of an IL-34 antisense oligonucleotide, or a pharmaceutically acceptable salt thereof, of any one of claims 1-18, or a compound of any one of claims 19-21.

23. A method of inhibiting inflammatory cytokine production in cells of a patient suffering from an inflammatory disease, the method comprising administering an effective amount of an IL-34 antisense oligonucleotide, or a pharmaceutically acceptable salt thereof, of any one of claims 1-18, or a compound of any one of claims 19-21.

24. A method of reducing or inhibiting an IL-34 mediated inflammatory response in cells of a patient suffering from an inflammatory disease, the method comprising administering an effective amount of an IL-34 antisense oligonucleotide, or a pharmaceutically acceptable salt thereof, of any one of claims 1-18, or a compound of any one of claims 19-21.

25. A method of treating an inflammatory disease associated with altered IL-34 expression in a patient in need thereof, the method comprising administering an effective amount of an IL-34 antisense oligonucleotide, or a pharmaceutically acceptable salt thereof, of any one of claims 1-18, or a compound of any one of claims 19-21.

26. A method of inhibiting IL-34-mediated macrophage colony-stimulating factor receptor (M-CSFR-1) signaling in cells of a patient suffering from an inflammatory disease, the method comprising administering an effective amount of an IL-34 antisense oligonucleotide, or a pharmaceutically acceptable salt thereof, of any one of claims 1-18, or a compound of any one of claims 19-21.

27. A method of reducing or eliminating a fibrotic stricture in a patient suffering from an inflammatory disease, the method comprising administering an effective amount of an IL-34 antisense oligonucleotide, or a pharmaceutically acceptable salt thereof, of any one of claims 1-18, or a compound of any one of claims 19-21.

28. The method of claim 27, wherein the fibrotic stricture is located in the intestine.

29. The method of any one of claims 22-28, wherein said inflammatory disease is selected from the group consisting of an inflammatory bowel disease, rheumatoid arthritis, psoriasis, osteoarthritis, diabetes (type I and II), tissue or organ rejection, multiple sclerosis, periodontal inflammation, periodontitis, pigmented villonodular synovitis, hepatitis, sinusitis, colon cancer, colorectal cancer, colitis-associated colon cancer, sporadic colorectal cancer, coronary artery disease, Sjogren's syndrome (SS), obesity, chronic inflammation, pulmonary sarcoidosis, skin lesions, a CNS inflammatory disease, or an autoimmune disease.

30. The method of claim 29, wherein said inflammatory disease is an inflammatory bowel disease.

31. The method of claim 30, wherein said inflammatory bowel disease is selected from the group consisting of Crohn's disease, gastroduodenal Crohn's disease, Crohn's (granulomatous) colitis, inflammatory Crohn's disease, fibrostricturing Crohn's disease, ulcerative colitis, collagenous colitis, lymphocytic colitis, ischaemic colitis, diversion colitis, Behcet's disease, microscopic colitis, ulcerative proctitis, proctosigmoiditis, jejunoileitis, left-sided colitis, pancolitis, ileocolitis, ileitis, and indeterminate colitis.

32. The method of claim 31, wherein said inflammatory bowel disease is inflammatory Crohn's disease.

33. The method of claim 31, wherein said inflammatory bowel disease is fibrostricturing Crohn's disease.

34. The method of any one of claims 22-33, wherein the IL-34 antisense oligonucleotide is administered topically, parenterally, orally, pulmonarily, intratracheally, intranasally, transdermally, or intraduodenally.

35. The method of claim 34, wherein the IL-34 antisense oligonucleotide is administered orally.

36. The method of any one of claims 22-35, wherein the patient is a human.

37. A pharmaceutically acceptable composition comprising an IL-34 antisense oligonucleotide of any one of claims 1-18, or a pharmaceutically acceptable salt thereof, or a compound of any one of claims 19-21, and a pharmaceutically acceptable excipient.

38. The pharmaceutical composition of claim 37, wherein the pharmaceutical composition is suitable for topical, parenteral, oral, pulmonary, intratracheal, intranasal, transdermal, or intraduodenal administration.

39. Use of an IL-34 antisense oligonucleotide of any one of claims 1-18, or a pharmaceutically acceptable salt thereof, or a compound of any one of claims 19-21, in the manufacture of a medicament for the treatment of an inflammatory disease.

40. The use of claim 39, wherein said inflammatory disease is selected from the group consisting of an inflammatory bowel disease, rheumatoid arthritis, psoriasis, osteoarthritis, diabetes (type I and II), tissue or organ rejection, multiple sclerosis, periodontal inflammation, periodontitis, pigmented villonodular synovitis, hepatitis, sinusitis, colon cancer, colorectal cancer, colitis-associated colon cancer, sporadic colorectal cancer, coronary artery disease, or Sjogren's syndrome (SS), obesity, chronic inflammation, pulmonary sarcoidosis, skin lesions, a CNS inflammatory disease, or an autoimmune disease.

41. The use of claim 40, wherein said inflammatory disease is an inflammatory bowel disease.

42. The use of claim 41, wherein the inflammatory bowel disease is selected from the group consisting of Crohn's disease, inflammatory Crohn's disease, fibrostricturing Crohn's disease, gastroduodenal Crohn's disease, Crohn's (granulomatous) colitis, ulcerative colitis, collagenous colitis, lymphocytic colitis, ischaemic colitis, diversion colitis, Behcet's disease, microscopic colitis, ulcerative proctitis, proctosigmoiditis, jejunoileitis, left-sided colitis, pancolitis, ileocolitis, ileitis, and indeterminate colitis.

43. The use of claim 42, wherein said inflammatory bowel disease is inflammatory Crohn's disease.

44. The use of claim 42, wherein said inflammatory bowel disease is fibrostricturing Crohn's disease.

45. A method of inhibiting IL-34 expression in a cell of a subject, the method comprising administering to the subject a pharmaceutically effective amount of a pharmaceutical preparation comprising an IL-34 antisense oligonucleotide, or a pharmaceutically acceptable salt thereof, of any one of claims 1-18, or a compound of any one of claims 19-21.

46. A method of inhibiting expression of one or more collagens in a cell of a subject, the method comprising administering to the subject a pharmaceutically effective amount of a pharmaceutical preparation comprising an IL-34 antisense oligonucleotide, or a pharmaceutically acceptable salt thereof, of any one of claims 1-18, or a compound of any one of claims 19-21.

47. The method of claim 45 or 46, wherein the cell is an intestinal cell.

48. The method of claim 47, wherein the cell is an intestinal stromal cell.

49. The method of claim 45 or 46, wherein the cell forms part of an intestinal fibrostricture.

50. The method of claim 45 or 46, wherein the subject is in need of treatment of a disease selected from inflammatory Crohn's disease or fibrostricturing Crohn's disease.

51. The method of claim 46, wherein the one or more collagens is selected from the group consisting of collagen 1A, collagen 3A, and a mixture thereof.

52. The method of claim 45 or 46, wherein the pharmaceutical preparation is administered orally.

53. The method of claim 45 or 46, wherein the subject is a human.

54. A method for preventing or treating fibrosis, the method comprising administering to a patient in need thereof a therapeutically effective amount of an IL-34 antisense oligonucleotide, or a pharmaceutically acceptable salt thereof, of any one of claims 1-18, or a compound of any one of claims 19-21.

55. The method of claim 54, wherein the fibrosis is intestinal fibrosis.

56. The method of claim 54, wherein the fibrosis is pulmonary fibrosis.

57. The method of claim 54, wherein the fibrosis is selected from the group consisting of renal fibrosis, cardiac fibrosis, endomyocardial fibrosis, myelofibrosis, retroperitoneal fibrosis, and nephrogenic systemic fibrosis.

58. A method of preventing or treating intestinal fibrosis, the method comprising administering to a patient in need thereof, a pharmaceutically effective amount of a pharmaceutical preparation comprising an IL-34 antisense oligonucleotide of any one of claims 1-18, or a pharmaceutically acceptable salt thereof, or a compound of any one of claims 19-21.

59. The method of claim 58, wherein the pharmaceutical preparation is administered orally.

60. The method of claim 58 or 59, wherein the patient is a human.

61. The method of any one of claims 58-60, wherein the patient is also suffering from Crohn's disease.

62. A method of preventing or treating pulmonary fibrosis, the method comprising administering to a patient in need thereof, a pharmaceutically effective amount of a pharmaceutical preparation comprising an IL-34 antisense oligonucleotide of any one of claims 1-18, or a pharmaceutically acceptable salt thereof, or a compound of any one of claims 19-21.

63. The method of claim 62, wherein the pharmaceutical preparation is administered orally.

64. The method of claim 62 or 63, wherein the patient is human.

65. An IL-34 antisense oligonucleotide of any one of claims 1-18, or a pharmaceutically acceptable salt thereof, or a compound of any one of claims 19-21 for use as a medicament.

66. An IL-34 antisense oligonucleotide of any one of claims 1-18, or a pharmaceutically acceptable salt thereof, or a compound of any one of claims 19-21 for use in the treatment of an inflammatory disease.

67. The IL-34 antisense oligonucleotide for use as claimed in claim 66, wherein said inflammatory disease is selected from the group consisting of an inflammatory bowel disease, rheumatoid arthritis, psoriasis, osteoarthritis, diabetes (type I and II), tissue or organ rejection, multiple sclerosis, periodontal inflammation, periodontitis, pigmented villonodular synovitis, hepatitis, sinusitis, colon cancer, colorectal cancer, colitis-associated colon cancer, sporadic colorectal cancer, coronary artery disease, or Sjogren's syndrome (SS), obesity, chronic inflammation, pulmonary sarcoidosis, skin lesions, a CNS inflammatory disease, or an autoimmune disease.

68. The IL-34 antisense oligonucleotide for use as claimed in claim 67, wherein said inflammatory disease is an inflammatory bowel disease.

69. The IL-34 antisense oligonucleotide for use as claimed in claim 68, wherein the inflammatory bowel disease is selected from the group consisting of Crohn's disease, inflammatory Crohn's disease, fibrostricturing Crohn's disease, gastroduodenal Crohn's disease, Crohn's (granulomatous) colitis, ulcerative colitis, collagenous colitis, lymphocytic colitis, ischaemic colitis, diversion colitis, Behçet's disease, microscopic colitis, ulcerative proctitis, proctosigmoiditis, jejunoileitis, left-sided colitis, pancolitis, ileocolitis, ileitis, and indeterminate colitis.

70. The IL-34 antisense oligonucleotide for use as claimed in claim 69, wherein the inflammatory bowel disease is inflammatory Crohn's disease.

71. The IL-34 antisense oligonucleotide for use as claimed in claim 69, wherein the inflammatory bowel disease is fibrostricturing Crohn's disease.

72. An IL-34 antisense oligonucleotide of any one of claims 1-18, or a pharmaceutically acceptable salt thereof, for use in the treatment of fibrosis.

73. The IL-34 antisense oligonucleotide for use as claimed in claim 72, wherein the fibrosis is intestinal fibrosis.

74. The IL-34 antisense oligonucleotide for use as claimed in claim 72, wherein the fibrosis is pulmonary fibrosis.

75. The IL-34 antisense oligonucleotide for use as claimed in claim 72, wherein the fibrosis is selected from the group consisting of renal fibrosis, cardiac fibrosis, endomyocardial fibrosis, myelofibrosis, retroperitoneal fibrosis, and nephrogenic systemic fibrosis.

76. An IL-34 inhibitor comprising a nucleotide sequence selected from the group consisting of:
5′-CTCACCAAGACCCACAG-3′ (SEQ ID NO:1), wherein at least one nucleoside is a chemically modified nucleoside and/or at least one linkage is a modified internucleoside linkage;
5′-GGCTTTGGGCCGCACCAGCT-3′ (SEQ ID NO:2), wherein at least one nucleoside is a chemically modified nucleoside and/or at least one linkage is a modified internucleoside linkage;
5′-CTTTGGGCCGCACCAGCTTC-3′ (SEQ ID NO:3), wherein at least one nucleoside is a chemically modified nucleoside and/or at least one linkage is a modified internucleoside linkage;
5′-TGGGCCGCACCAGCTTCAGG-3′ (SEQ ID NO:4), wherein at least one nucleoside is a chemically modified nucleoside and/or at least one linkage is a modified internucleoside linkage;
5′-TCCATGACCCGGAAGCAGTT-3′ (SEQ ID NO:5), wherein at least one nucleoside is a chemically modified nucleoside and/or at least one linkage is a modified internucleoside linkage; and 5′-TGTTTCATGTACTGAAG-3′ (SEQ ID NO:6), wherein at least one nucleoside is a chemically modified nucleoside and/or at least one linkage is a modified internucleoside linkage,
or
a pharmaceutically acceptable salt thereof.

77. The IL-34 inhibitor of claim 76, wherein the nucleotide sequence is SEQ ID NO:3, and wherein at least one cytidine is a chemically modified.

78. The IL-34 inhibitor of claim 77, wherein the nucleotide sequence is 5′-CTTTGGGCXGCACCAGCTTC-3′ (SEQ ID NO:7), and wherein X is 5-methylcytidine.

79. The IL-34 inhibitor of claim 76, wherein the nucleotide sequence is SEQ ID NO:5, and wherein at least one cytidine is chemically modified; optionally wherein the cytidine at position 10 of SEQ ID NO:5 is chemically modified and the nucleotide sequence is 5′-TCCATGACCXGGAAGCAGTT-3′ (SEQ ID NO:8), and wherein X is 5-methylcytidine.

80. The IL-34 inhibitor of any one of claims 76-79, wherein the antisense oligonucleotide comprises one or more locked nucleic acids (LNA).

81. The IL-34 inhibitor of claim 76-80, wherein each of the nucleotides of the antisense oligonucleotide comprises a locked nucleic acid (LNA).

82. The IL-34 inhibitor of any one of claims 76-81, wherein the IL-34 inhibitor is an IL-34 siRNA, or a pharmaceutically acceptable salt thereof.

83. The IL-34 inhibitor of claim 82, wherein at least one internucleoside linkage of the sequence is selected from the group consisting of a phosphorothioate linkage, a phosphorodithioate linkage, a phosphotriester linkage, an alkylphosphonate linkage, an aminoalkylphosphotriester linkage, an alkylene phosphonate linkage, a phosphinate linkage, a phosphoramidate linkage, and an aminoalkylphosphoramidate linkage, a thiophosphoramidate linkage, thionoalkylphosphonate linkage, a thionoalkylphosphotriester linkage, a thiophosphate linkage, a selenophosphate linkage, and a boranophosphate linkage.

84. The IL-34 inhibitor of claim 82 or 83, wherein at least one internucleoside linkage of the sequence is a phosphorothioate linkage.

85. The IL-34 inhibitor any one of claims 82-84, wherein all internucleoside linkages of the sequence are phosphorothioate linkages.

86. The IL-34 inhibitor of any one of claims 82-85, wherein one or more cytidines are replaced with 5-methylcytidine.

87. The IL-34 inhibitor of any one of claims 82-86 wherein the IL-34 siRNA is 17, 18, 19, 20, 21, 22, 23, 24, 25, 26, 27, 28, 29, 30, 20-25, 20-30, or 25-30 nucleotides in length.

88. The IL-34 inhibitor of claim 87, wherein the IL-34 siRNA is from 20-25 nucleotides in length.

89. The IL-34 inhibitor of any one of claims 82-86, wherein the IL-34 siRNA is no more than 20, 25, or 30 nucleotides in length.

90. A method of treating an inflammatory disease, the method comprising administering to a patient in need thereof an effective amount of an IL-34 inhibitor, or a pharmaceutically acceptable salt thereof, of any one of claims 76-89.

91. A method of inhibiting inflammatory cytokine production in cells of a patient suffering from an inflammatory disease, the method comprising administering an effective amount of an IL-34 inhibitor, or a pharmaceutically acceptable salt thereof, of any one of claims 76-89.

92. A method of reducing or inhibiting an IL-34 mediated inflammatory response in cells of a patient suffering from an inflammatory disease, the method comprising administering an effective amount of an IL-34 inhibitor, or a pharmaceutically acceptable salt thereof, of any one of claims 76-89.

93. A method of treating an inflammatory disease associated with altered IL-34 expression in a patient in need thereof, the method comprising administering an effective amount of an IL-34 inhibitor, or a pharmaceutically acceptable salt thereof, of any one of claims 76-89.

94. A method of inhibiting IL-34-mediated macrophage colony-stimulating factor receptor (M-CSFR-1) signaling in cells of a patient suffering from an inflammatory disease, the method comprising administering an effective amount of an IL-34 inhibitor, or a pharmaceutically acceptable salt thereof, of any one of claims 76-89.

95. A method of reducing or eliminating a fibrotic stricture in a patient suffering from an inflammatory disease, the method comprising administering an effective amount of an IL-34 inhibitor, or a pharmaceutically acceptable salt thereof, of any one of claims 76-89.

96. The method of claim 95, wherein the fibrotic stricture is located in the intestine.

97. The method of any one of claims 90-96, wherein said inflammatory disease is selected from the group consisting of an inflammatory bowel disease, rheumatoid arthritis, psoriasis, osteoarthritis, diabetes (type I and II), tissue or organ rejection, multiple sclerosis, periodontal inflammation, periodontitis, pigmented villonodular synovitis, hepatitis, sinusitis, colon cancer, colorectal cancer, colitis-associated colon cancer, sporadic colorectal cancer, coronary artery disease, Sjogren's syndrome (SS), obesity, chronic inflammation, pulmonary sarcoidosis, skin lesions, a CNS inflammatory disease, or an autoimmune disease.

98. The method of claim 97, wherein said inflammatory disease is an inflammatory bowel disease.

99. The method of claim 98, wherein said inflammatory bowel disease is selected from the group consisting of Crohn's disease, gastroduodenal Crohn's disease, Crohn's (granulomatous) colitis, inflammatory Crohn's disease, fibrostricturing Crohn's disease, ulcerative colitis, collagenous colitis, lymphocytic colitis, ischaemic colitis, diversion colitis, Behcet's disease, microscopic colitis, ulcerative proctitis, proctosigmoiditis, jejunoileitis, left-sided colitis, pancolitis, ileocolitis, ileitis, and indeterminate colitis.

100. The method of claim 99, wherein said inflammatory bowel disease is inflammatory Crohn's disease.

101. The method of claim 99, wherein said inflammatory bowel disease is fibrostricturing Crohn's disease.

102. The method of any one of claims 90-101, wherein the IL-34 inhibitor is administered topically, parenterally, orally, pulmonarily, intratracheally, intranasally, transdermally, or intraduodenally.

103. The method of claim 102, wherein the IL-34 inhibitor is administered orally.

104. The method of any one of claims 90-103, wherein the patient is a human.

105. A pharmaceutically acceptable composition comprising an IL-34 inhibitor of any one of claims 76-89, or a pharmaceutically acceptable salt thereof, and a pharmaceutically acceptable excipient.

106. The method of claim 105, wherein the pharmaceutical composition is suitable for topical, parenteral, oral, pulmonary, intratracheal, intranasal, transdermal, or intraduodenal administration.

107. Use of an IL-34 inhibitor of any one of claims 76-89, or a pharmaceutically acceptable salt thereof, in the manufacture of a medicament for the treatment of an inflammatory disease.

108. The use of claim 107, wherein said inflammatory disease is selected from the group consisting of an inflammatory bowel disease, rheumatoid arthritis, psoriasis, osteoarthritis, diabetes (type I and II), tissue or organ rejection, multiple sclerosis, periodontal inflammation, periodontitis, pigmented villonodular synovitis, hepatitis, sinusitis, colon cancer, colorectal cancer, colitis-associated colon cancer, sporadic colorectal cancer, coronary artery disease, or Sjogren's syndrome (SS), obesity, chronic inflammation, pulmonary sarcoidosis, skin lesions, a CNS inflammatory disease, or an autoimmune disease.

109. The use of claim 108, wherein said inflammatory disease is an inflammatory bowel disease.

110. The use of claim 109, wherein the inflammatory bowel disease is selected from the group consisting of Crohn's disease, inflammatory Crohn's disease, fibrostricturing Crohn's disease, gastroduodenal Crohn's disease, Crohn's (granulomatous) colitis, ulcerative colitis, collagenous colitis, lymphocytic colitis, ischaemic colitis, diversion colitis, Behçet's disease, microscopic colitis, ulcerative proctitis, proctosigmoiditis, jejunoileitis, left-sided colitis, pancolitis, ileocolitis, ileitis, and indeterminate colitis.

111. The use of claim 110, wherein said inflammatory bowel disease is inflammatory Crohn's disease.

112. The use of claim 110, wherein said inflammatory bowel disease is fibrostricturing Crohn's disease.

113. A method of inhibiting IL-34 expression in a cell of a subject, the method comprising administering to the subject a pharmaceutically effective amount of a pharmaceutical preparation comprising an IL-34 inhibitor, or a pharmaceutically acceptable salt thereof, of any one of claims 76-89.

114. A method of inhibiting expression of one or more collagens in a cell of a subject, the method comprising administering to the subject a pharmaceutically effective amount of a pharmaceutical preparation comprising an IL-34 inhibitor, or a pharmaceutically acceptable salt thereof, of any one of claims 76-89.

115. The method of claim 113 or 114, wherein the cell is an intestinal cell.

116. The method of claim 115, wherein the cell is an intestinal stromal cell.

117. The method of claim 113 or 114, wherein the cell forms part of an intestinal fibrostricture.

118. The method of claim 113 or 114, wherein the subject is in need of treatment of a disease selected from inflammatory Crohn's disease or fibrostricturing Crohn's disease.

119. The method of claim 114, wherein the one or more collagens is selected from the group consisting of collagen 1A, collagen 3A, and a mixture thereof.

120. The method of claim 113 or 114, wherein the pharmaceutical preparation is administered orally.

121. The method of claim 113 or 114, wherein the subject is a human.

122. A method for preventing or treating fibrosis, the method comprising administering to a patient in need thereof a therapeutically effective amount of an IL-34 inhibitor, or a pharmaceutically acceptable salt thereof, of any one of claims 76-89.

123. The method of claim 122, wherein the fibrosis is intestinal fibrosis.

124. A method of preventing or treating intestinal fibrosis, the method comprising administering to a patient in need thereof, a pharmaceutically effective amount of a pharmaceutical preparation comprising an IL-34 inhibitor of any one of claims 76-89, or a pharmaceutically acceptable salt thereof.

125. The method of claim 124, wherein the pharmaceutical preparation is administered orally.

126. The method of claim 124 or 125, wherein the patient is a human.

127. The method of any one of claims 124-126, wherein the patient is also suffering from Crohn's disease.

128. An IL-34 inhibitor of any one of claims 76-89, or a pharmaceutically acceptable salt thereof, for use as a medicament.

129. An IL-34 inhibitor of any one of claims 76-89, or a pharmaceutically acceptable salt thereof, for use in the treatment of an inflammatory disease.

130. The IL-34 inhibitor for use as claimed in claim 129, wherein said inflammatory disease is selected from the group consisting of an inflammatory bowel disease, rheumatoid arthritis, psoriasis, osteoarthritis, diabetes (type I and II), tissue or organ rejection, multiple sclerosis, periodontal inflammation, periodontitis, pigmented villonodular synovitis, hepatitis, sinusitis, colon cancer, colorectal cancer, colitis-associated colon cancer, sporadic colorectal cancer, coronary artery disease, or Sjogren's syndrome (SS), obesity, chronic inflammation, pulmonary sarcoidosis, skin lesions, a CNS inflammatory disease, or an autoimmune disease.

131. The IL-34 inhibitor for use as claimed in claim 130, wherein said inflammatory disease is an inflammatory bowel disease.

132. The IL-34 inhibitor for use as claimed in claim 131, wherein the inflammatory bowel disease is selected from the group consisting of Crohn's disease, inflammatory Crohn's disease, fibrostricturing Crohn's disease, gastroduodenal Crohn's disease, Crohn's (granulomatous) colitis, ulcerative colitis, collagenous colitis, lymphocytic colitis, ischaemic colitis, diversion colitis, Behçet's disease, microscopic colitis, ulcerative proctitis, proctosigmoiditis, jejunoileitis, left-sided colitis, pancolitis, ileocolitis, ileitis, and indeterminate colitis.

133. The IL-34 inhibitor for use as claimed in claim 132, wherein the inflammatory bowel disease is inflammatory Crohn's disease.

134. The IL-34 inhibitor for use as claimed in claim 132, wherein the inflammatory bowel disease is fibrostricturing Crohn's disease.

135. An IL-34 inhibitor of any one of claims 76-89, or a pharmaceutically acceptable salt thereof, for use in the treatment of fibrosis.

136. The IL-34 inhibitor for use as claimed in claim 135, wherein the fibrosis is intestinal fibrosis.


SEQUENCE LISTING (39 sequences)

SEQ ID NO: 1 - DNA, 17 bp, Artificial Sequence
  source: /note=“Description of Artificial Sequence Synthetic oligonucleotide”
ctcaccaagacccacag

SEQ ID NO: 2 - DNA, 20 bp, Artificial Sequence
  source: /note=“Description of Artificial Sequence Synthetic oligonucleotide”
ggctttgggccgcaccagct

SEQ ID NO: 3 - DNA, 20 bp, Artificial Sequence
  source: /note=“Description of Artificial Sequence Synthetic oligonucleotide”
ctttgggccgcaccagcttc

SEQ ID NO: 4 - DNA, 20 bp, Artificial Sequence
  source: /note=“Description of Artificial Sequence Synthetic oligonucleotide”
tgggccgcaccagcttcagg

SEQ ID NO: 5 - DNA, 20 bp, Artificial Sequence
  source: /note=“Description of Artificial Sequence Synthetic oligonucleotide”
tccatgacccggaagcagtt

SEQ ID NO: 6 - DNA, 17 bp, Artificial Sequence
  source: /note=“Description of Artificial Sequence Synthetic oligonucleotide”
tgtttcatgtactgaag

SEQ ID NO: 7 - DNA, 20 bp, Artificial Sequence
  source: /note=“Description of Artificial Sequence Synthetic oligonucleotide”
ctttgggccgcaccagcttc

SEQ ID NO: 8 - DNA, 20 bp, Artificial Sequence
  source: /note=“Description of Artificial Sequence Synthetic oligonucleotide”
tccatgacccggaagcagtt

SEQ ID NO: 9 - DNA, 20 bp, Artificial Sequence
  source: /note=“Description of Artificial Sequence Synthetic oligonucleotide”
ctttgggccgcaccagcttc

SEQ ID NO: 10 - DNA, 20 bp, Artificial Sequence
  source: /note=“Description of Artificial Sequence Synthetic oligonucleotide”
ctttgggccgcaccagcttc

SEQ ID NO: 11 - DNA, 20 bp, Artificial Sequence
  source: /note=“Description of Artificial Sequence Synthetic oligonucleotide”
ctttgggccgcaccagcttc

SEQ ID NO: 12 - DNA, 20 bp, Artificial Sequence
  source: /note=“Description of Artificial Sequence Synthetic oligonucleotide”
ctttgggccgcaccagcttc

SEQ ID NO: 13 - DNA, 15 bp, Artificial Sequence
  source: /note=“Description of Artificial Sequence Synthetic oligonucleotide”
ggccgcaccagcttc

SEQ ID NO: 14 - DNA, 16 bp, Artificial Sequence
  source: /note=“Description of Artificial Sequence Synthetic oligonucleotide”
ctttgggccgcaccag

SEQ ID NO: 15 - DNA, 16 bp, Artificial Sequence
  source: /note=“Description of Artificial Sequence Synthetic oligonucleotide”
tgacccggaagcagtt

SEQ ID NO: 16 - DNA, 20 bp, Artificial Sequence
  source: /note=“Description of Artificial Sequence Synthetic oligonucleotide”
ctttgggccgcaccagcttc

SEQ ID NO: 17 - DNA, 20 bp, Artificial Sequence
  source: /note=“Description of Artificial Sequence Synthetic oligonucleotide”
tccatgacccggaagcagtt

SEQ ID NO: 18 - DNA, 20 bp, Artificial Sequence
  source: /note=“Description of Artificial Sequence Synthetic oligonucleotide”
ctttgggccgcaccagcttc

SEQ ID NO: 19 - DNA, 20 bp, Artificial Sequence
  source: /note=“Description of Artificial Sequence Synthetic oligonucleotide”
tccatgacccggaagcagtt

SEQ ID NO: 20 - DNA, 20 bp, Artificial Sequence
  source: /note=“Description of Artificial Sequence Synthetic oligonucleotide”
ctttgggccgcaccagcttc

SEQ ID NO: 21 - DNA, 20 bp, Artificial Sequence
  source: /note=“Description of Artificial Sequence Synthetic oligonucleotide”
tccatgacccggaagcagtt

SEQ ID NO: 22 - DNA, 20 bp, Artificial Sequence
  source: /note=“Description of Artificial Sequence Synthetic oligonucleotide”
ctttgggccgcaccagcttc

SEQ ID NO: 23 - DNA, 20 bp, Artificial Sequence
  source: /note=“Description of Artificial Sequence Synthetic oligonucleotide”
tccatgacccggaagcagtt

SEQ ID NO: 24 - DNA, 1793 bp, Homo sapiens
catcagacgggaagcctggactgtgggttgggggcagcctcagcctctccaacctggcacccactgcccgtggcccttaggcacctgcttggggtcctggagccccttaaggccaccagcaaatcctaggagaccgagtcttggcacgtgaacagagccagatttcacactgagcagctgcagtcggagaaatcagagaaagcgtcacccagccccagattccgaggggcctgccagggactctctcctcctgctccttggaaaggaagaccccgaaagacccccaagccaccggctcagacctgcttctgggctgccatgggacttgcggccaccgccccccggctgtcctccacgctgccgggcagataagggcagctgctgcccttggggcacctgctcactcccgcagcccagccactcctccagggccagcccttccctgactgagtgaccacctctgctgccccgaggccatgtaggccgtgcttaggcctctgtggacacactgctggggacggcgcctgagctctcagggggacgaggaacaccaccatgccccggggcttcacctggctgcgctatcttgggatcttccttggcgtggccttggggaatgagcctttggagatgtggcccttgacgcagaatgaggagtgcactgtcacgggttttctgcgggacaagctgcagtacaggagccgacttcagtacatgaaacactacttccccatcaactacaagatcagtgtgccttacgagggggtgttcagaatcgccaacgtcaccaggctgagggcccaggtgagcgagcgggagctgcggtatctgtgggtcttggtgagcctcagtgccactgagtcggtgcaggacgtgctgctcgagggccacccatcctggaagtacctgcaggaggtggagacgctgctgctgaatgtccagcagggcctcacggatgtggaggtcagccccaaggtggaatccgtgttgtccctcttgaatgccccagggccaaacctgaagctggtgcggcccaaagccctgctggacaactgcttccgggtcatggagctgctgtactgctcctgctgtaaacaaagctccgtcctaaactggcaggactgtgaggtgccaagtcctcagtcttgcagcccagagccctcattgcagtatgcggccacccagctgtaccctccgcccccgtggtcccccagctccccgcctcactccacgggctcggtgaggccggtcagggcacagggcgagggcctcttgccctgagcaccctggatggtgactgcggataggggcagccagaccagctcccacaggagttcaactgggtctgagacttcaaggggtggtggtgggagccccccttgggagaggacccctgggaagggtgtttttcctttgagggggattctgtgccacagcagggctcagcttcctgccttccatagctgtcatggcctcacctggagcggaggggacctggggacctgaaggtggatggggacacagctcctggcttctcctggtgctgccctcactgtccccccgcctaaagggggtactgagcctcctgtggcccgcagcagtgagggcacagctgtgggttgcaggggagacagccagcacggcgtggccattctatgaccccccagcctggcagactggggagctgggggcagagggcggtgccaagtgccacatcttgccatagtggatgctcttccagtttcttttttctattaaacaccccacttcctttggaaaaaaaaaaaaaaaaa

SEQ ID NO: 25 - DNA, 1779 bp, Homo sapiens
ctcgaagctcggcgtctgtgatggtctacggggctttcgaggtgatcaggcagcgtcagtcttcagccgctaagccgagaaggagtcagtcagagagccttgggccagagttccaggggctctgggagtggctgccagagccagatttcacactgagcagctgcagtcggagaaatcagagaaagcgtcacccagccccagattccgaggggcctgccagggactctctcctcctgctccttggaaaggaagaccccgaaagacccccaagccaccggctcagacctgcttctgggctgccatgggacttgcggccaccgccccccggctgtcctccacgctgccgggcagataagggcagctgctgcccttggggcacctgctcactcccgcagcccagccactcctccagggccagcccttccctgactgagtgaccacctctgctgccccgaggccatgtaggccgtgcttaggcctctgtggacacactgctggggacggcgcctgagctctcagggggacgaggaacaccaccatgccccggggcttcacctggctgcgctatcttgggatcttccttggcgtggccttggggaatgagcctttggagatgtggcccttgacgcagaatgaggagtgcactgtcacgggttttctgcgggacaagctgcagtacaggagccgacttcagtacatgaaacactacttccccatcaactacaagatcagtgtgccttacgagggggtgttcagaatcgccaacgtcaccaggctgcagagggcccaggtgagcgagcgggagctgcggtatctgtgggtcttggtgagcctcagtgccactgagtcggtgcaggacgtgctgctcgagggccacccatcctggaagtacctgcaggaggtggagacgctgctgctgaatgtccagcagggcctcacggatgtggaggtcagccccaaggtggaatccgtgttgtccctcttgaatgccccagggccaaacctgaagctggtgcggcccaaagccctgctggacaactgcttccgggtcatggagctgctgtactgctcctgctgtaaacaaagctccgtcctaaactggcaggactgtgaggtgccaagtcctcagtcttgcagcccagagccctcattgcagtatgcggccacccagctgtaccctccgcccccgtggtcccccagctccccgcctcactccacgggctcggtgaggccggtcagggcacagggcgagggcctcttgccctgagcaccctggatggtgactgcggataggggcagccagaccagctcccacaggagttcaactgggtctgagacttcaaggggtggtggtgggagccccccttgggagaggacccctgggaagggtgtttttcctttgagggggattctgtgccacagcagggctcagcttcctgccttccatagctgtcatggcctcacctggagcggaggggacctggggacctgaaggtggatggggacacagctcctggcttctcctggtgctgccctcactgtccccccgcctaaagggggtactgagcctcctgtggcccgcagcagtgagggcacagctgtgggttgcaggggagacagccagcacggcgtggccattctatgaccccccagcctggcagactggggagctgggggcagagggcggtgccaagtgccacatcttgccatagtggatgctcttccagtttcttttttctattaaacaccccacttcctttggaaaaaaaaaaaaaaaaa

SEQ ID NO: 26 - DNA, 1796 bp, Homo sapiens
catcagacgggaagcctggactgtgggttgggggcagcctcagcctctccaacctggcacccactgcccgtggcccttaggcacctgcttggggtcctggagccccttaaggccaccagcaaatcctaggagaccgagtcttggcacgtgaacagagccagatttcacactgagcagctgcagtcggagaaatcagagaaagcgtcacccagccccagattccgaggggcctgccagggactctctcctcctgctccttggaaaggaagaccccgaaagacccccaagccaccggctcagacctgcttctgggctgccatgggacttgcggccaccgccccccggctgtcctccacgctgccgggcagataagggcagctgctgcccttggggcacctgctcactcccgcagcccagccactcctccagggccagcccttccctgactgagtgaccacctctgctgccccgaggccatgtaggccgtgcttaggcctctgtggacacactgctggggacggcgcctgagctctcagggggacgaggaacaccaccatgccccggggcttcacctggctgcgctatcttgggatcttccttggcgtggccttggggaatgagcctttggagatgtggcccttgacgcagaatgaggagtgcactgtcacgggttttctgcgggacaagctgcagtacaggagccgacttcagtacatgaaacactacttccccatcaactacaagatcagtgtgccttacgagggggtgttcagaatcgccaacgtcaccaggctgcagagggcccaggtgagcgagcgggagctgcggtatctgtgggtcttggtgagcctcagtgccactgagtcggtgcaggacgtgctgctcgagggccacccatcctggaagtacctgcaggaggtggagacgctgctgctgaatgtccagcagggcctcacggatgtggaggtcagccccaaggtggaatccgtgttgtccctcttgaatgccccagggccaaacctgaagctggtgcggcccaaagccctgctggacaactgcttccgggtcatggagctgctgtactgctcctgctgtaaacaaagctccgtcctaaactggcaggactgtgaggtgccaagtcctcagtcttgcagcccagagccctcattgcagtatgcggccacccagctgtaccctccgcccccgtggtcccccagctccccgcctcactccacgggctcggtgaggccggtcagggcacagggcgagggcctcttgccctgagcaccctggatggtgactgcggataggggcagccagaccagctcccacaggagttcaactgggtctgagacttcaaggggtggtggtgggagccccccttgggagaggacccctgggaagggtgtttttcctttgagggggattctgtgccacagcagggctcagcttcctgccttccatagctgtcatggcctcacctggagcggaggggacctggggacctgaaggtggatggggacacagctcctggcttctcctggtgctgccctcactgtccccccgcctaaagggggtactgagcctcctgtggcccgcagcagtgagggcacagctgtgggttgcaggggagacagccagcacggcgtggccattctatgaccccccagcctggcagactggggagctgggggcagagggcggtgccaagtgccacatcttgccatagtggatgctcttccagtttcttttttctattaaacaccccacttcctttggaaaaaaaaaaaaaaaaa

SEQ ID NO: 27 - DNA, 1747 bp, Mus musculus
tctagaaacaaatctgagctgctgtagcgtgtgtcccccgccctccgagaagactgtaggctgaacacacaacagcctgtcccacacagcatttactgccacagcccccaaggcacctgctccaggccttgagctgctcaaagcttccggcgacggcgactgtagaagcccacatcctggcaggtgagcagagctagacttcacgctgagcagctgcagccgaagaaaccagagaaagcgccacccagccccagattccaagaggcctgcctgctggggactcccttctcttcttcctcctcctcctcttcctcctcctgagaccctcaaagtcactgtctcacaccagttcctgagctgcaatgggactggcagccactgccccctggctgtcctccgccctgccaagcagatagggcggtagctaccccaggagtatctgccccctctcagcctggaccagccctttctggacttggtggccacctttgctgacctaaggccatgtagtccctggctcaggcctctggacacacttctggggacagtgcctctgctctcagggggcacccagtgcaccaccatgccctggggactcgcctggctatactgtcttgggatcctacttgacgtggctttgggaaacgagaatttggagatatggactctgacccaagataaggagtgtgaccttacaggctaccttcggggcaagctgcagtacaagaaccggcttcagtacatgaaacattacttccccatcaactacaggattgctgtgccttatgagggggtactcagagtggccaacatcacaaggctgcagaaggctcacgtgagtgagcgagagcttcggtacctgtgggtcttggtgagtctcaatgccactgagtctgtgatggatgtacttctcgagggccacccgtcctggaagtatctacaggaggttcagacattgctggagaacgtacagcggagcctcatggatgtggagattggccctcacgtggaagctgtgttatctcttctgagtactccaggcctaagcctgaagctggtgcggcccaaagccttgctggacaactgcttccgggtcatggaactgctgtactgttcttgctgtaaacaaagccccatcttaaaatggcaggactgcgagctgcccaggctccatccccacagtccggggtccttgatgcaatgtacagctacaaatgtgtaccctttgtctcggcagacccccacctccctgcccggatccccaagctcaagccatggctcgttgccctgagcaatctatgtattgaccctgggtcgggccactggatgcatttcagaaactggcctggatctgagaccttcctggatggtgggtaggtagcccctttaggaaaggccttcagggaaaggcctcctgccttctcttccacacccaggcttcctgccttctggtggtatcacggagcctcaggggagggatgtggaggggagggctggttgctatcccttacagccacctctgcttgtggcccggtccgaaatctgcaaacactgttgccccacaggcgggtggccaagaacgacggggtgcctcctaggatcccaggacttactggggcttttcagttgtgctgactgtggggtgctgggccgaggaccaaccaaatgccgtatcttgccatgacgatgctcttctggttcctttttctattaaatggccatttatttggtttggcttgcaaaaaaa

SEQ ID NO: 28 - DNA, 1865 bp, Mus musculus
tctagaaacaaatctgagctgctgtagcgtgtgtcccccgccctccgagaagactgtaggctgaacacacaacagcctgtcccacacagcatttactgccacagcccccaaggcacctgctccaggccttgagctgctcaaagcttccggcgacggcgactgtagaagcccacatcctggcaggtgagcagagctagacttcacgctgagcagctgcagccgaagaaaccagagaaagcgccacccagccccagattccaagaggcctgcctgctggggactcccttctcttcttcctcctcctcctcttcctcctcctgagaccctcaaagtcactgtctcacaccagttcctgagctgcaatgggactggcagccactgccccctggctgtcctccgccctgccaagcagatagggcggtagctaccccaggagtatctgccccctctcagcctggaccagccctttctggacttggtggccacctttgctgacctaaggccatgtagtccctggctcaggcctctggacacacttctggggacagtgcctctgctctcagggggcacccagtgcaccaccatgccctggggactcgcctggctatactgtcttgggatcctacttgacgtggctttgggaaacgagaatttggagatatggactctgacccaagataaggagtgtgaccttacaggctaccttcggggcaagctgcagtacaagaaccggcttcagtacatgaaacattacttccccatcaactacaggattgctgtgccttatgagggggtactcagagtggccaacatcacaaggctgcagaaggctcacgtgagtgagcgagagcttcggtacctgtgggtcttggtgagtctcaatgccactgagtctgtgatggatgtacttctcgagggccacccgtcctggaagtatctacaggaggttcagacattgctggagaacgtacagcggagcctcatggccgttggtgtacacctgccgggacacgtacttgtgaccctgctcagccagctgcctggcctccccagcccatgggccagatcatttgacaccagctgggagcttctgatgatgaaaggatgtggagattggccctcacgtggaagctgtgttatctcttctgagtactccaggcctaagcctgaagctggtgcggcccaaagccttgctggacaactgcttccgggtcatggaactgctgtactgttcttgctgtaaacaaagccccatcttaaaatggcaggactgcgagctgcccaggctccatccccacagtccggggtccttgatgcaatgtacagctacaaatgtgtaccctttgtctcggcagacccccacctccctgcccggatccccaagctcaagccatggctcgttgccctgagcaatctatgtattgaccctgggtcgggccactggatgcatttcagaaactggcctggatctgagaccttcctggatggtgggtaggtagcccctttaggaaaggccttcagggaaaggcctcctgccttctcttccacacccaggcttcctgccttctggtggtatcacggagcctcaggggagggatgtggaggggagggctggttgctatcccttacagccacctctgcttgtggcccggtccgaaatctgcaaacactgttgccccacaggcgggtggccaagaacgacggggtgcctcctaggatcccaggacttactggggcttttcagttgtgctgactgtggggtgctgggccgaggaccaaccaaatgccgtatcttgccatgacgatgctcttctggttcctttttctattaaatggccatttatttggtttggcttgcaaaaaaa

SEQ ID NO: 29 - DNA, 20 bp, Artificial Sequence
  source: /note=“Description of Artificial Sequence Synthetic primer”
acaggagccgacttcagtac

SEQ ID NO: 30 - DNA, 20 bp, Artificial Sequence
  source: /note=“Description of Artificial Sequence Synthetic primer”
accaagacccacagataccg

SEQ ID NO: 31 - DNA, 27 bp, Artificial Sequence
  source: /note=“Description of Artificial Sequence Synthetic primer”
aagatgacccagatcatgtttgagacc

SEQ ID NO: 32 - DNA, 20 bp, Artificial Sequence
  source: /note=“Description of Artificial Sequence Synthetic primer”
agccagtccagacgcaggat

SEQ ID NO: 33 - DNA, 15 bp, Artificial Sequence
  source: /note=“Description of Artificial Sequence Synthetic oligonucleotide”
aacacgtctatacgc

SEQ ID NO: 34 - DNA, 20 bp, Artificial Sequence
  source: /note=“Description of Artificial Sequence Synthetic primer”
ctgctcaactttctgcgaag

SEQ ID NO: 35 - DNA, 20 bp, Artificial Sequence
  source: /note=“Description of Artificial Sequence Synthetic primer”
ctcatctccacataggtgtc

SEQ ID NO: 36 - DNA, 20 bp, Artificial Sequence
  source: /note=“Description of Artificial Sequence Synthetic primer”
ggacacagaggtttcagtgg

SEQ ID NO: 37 - DNA, 20 bp, Artificial Sequence
  source: /note=“Description of Artificial Sequence Synthetic primer”
ggacacagaggtttcagtgg

SEQ ID NO: 38 - DNA, 21 bp, Artificial Sequence
  source: /note=“Description of Artificial Sequence Synthetic primer”
ggagaatgttgtgcagtttgc

SEQ ID NO: 39 - DNA, 21 bp, Artificial Sequence
  source: /note=“Description of Artificial Sequence Synthetic primer”
ggagaatgttgtgcagtttgc